SEQUENCE LISTING (5 sequences)

SEQ ID NO: 1 - protein, 2136 aa, Homo sapiens
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 2 - DNA, 9612 bp, Homo sapiens
actccctgaggtctgtcctggggagacactgctgctccggggggctgacctggcggggagtggccgcgcagtctgctccggcgccgctttgtgcgcgcagccgctggcccctctactcccgggtctgccccccgggacacccctctgcctcgcccaagtcatgcagccctacctgcctctccactgtggacctttgggaaccgactcctcacctcgggggctcgggccttgactgtgctgggagccggtaggcgtcctccgcgacccgcccgcgcccctcgcgcccgccggggccccgggctccaaagttgtggggaccggcgcgagttggaaagtttgcccgagggctggtgcaggcttggagctgggggccgtgcgctgccctgggaatgtgacccggccagcgaccaaaaccttgtgtgactgagctgaagagcagtgcatccagattctcctcagaagtgagactttccaaaggaccaatgactctgtttcctgcgccctttcattttttcctactctgtagctatgtctcgatcccgccatgcaaggccttccagattagtcaggaaggaagatgtaaacaaaaaaaagaaaaacagccaactacgaaagacaaccaagggagccaacaaaaatgtggcatcagtcaagactttaagccctggaaaattaaagcaattaattcaagaaagagatgttaagaaaaaaacagaacctaaaccacccgtgccagtcagaagccttctgacaagagctggagcagcacgcatgaatttggataggactgaggttctttttcagaacccagagtccttaacctgcaatgggtttacaatggcgctacgaagcacctctcttagcaggcgactctcccaacccccactggtcgtagccaaatccaaaaaggttccactttctaagggtttagaaaagcaacatgattgtgattataagatactccctgctttgggagtaaagcactcagaaaatgattcggttccaatgcaagacacccaagtccttcctgatatagagactctaattggtgtacaaaatccctctttacttaaaggtaagagccaagagacaactcagttttggtcccaaagagttgaggattccaagatcaatatccctacccacagtggccctgcagctgagatccttcctgggccactggaagggacacgctgtggtgaaggactattctctgaagagacattgaatgataccagtggttccccaaaaatgtttgctcaggacacagtgtgtgctccttttccccaaagagcaacccccaaagttacctctcaaggaaaccccagcattcagttagaagagttgggttcacgagtagaatctcttaagttatctgattcttacctggatcccattaaaagtgaacatgattgctaccccacctccagtcttaataaggttatacctgacttgaaccttagaaactgcttggctcttggtgggtctacgtctcctacctctgtaataaaattcctcttggcaggctcaaaacaagcgacccttggtgctaaaccagatcatcaagaggccttcgaagctactgcaaatcaacaggaagtttctgataccacctctttcctaggacaggcctttggtgctatcccacatcaatgggaacttcctggtgctgacccagttcatggtgaggccctgggtgagaccccagatctaccagagattcctggtgctattccagtccaaggagaggtctttggtactattttagaccaacaagaaactcttggtatgagtgggagtgttgtcccagacttgcctgtcttccttcctgttcctccaaatccaattgctacctttaatgctccttccaaatggcctgagccccaaagcactgtctcatatggacttgcagtccagggtgctatacagattttgcctttgggctcaggacacactcctcaatcatcatcaaactcagagaaaaattcattacctccagtaatggctataagcaatgtagaaaatgagaagcaggttcatataagcttcctgccagctaacactcaggggttcccattagcccctgagagaggactcttccatgcttcactgggtatagcccaactctctcaggctggtcctagcaaatcagacagagggagctcccaggtcagtgtaaccagcacagttcatgttgtcaacaccacagtggtgactatgccagtgccaatggtcagtacctcctcttcttcctataccactttgctaccgactttggaaaagaagaaaagaaagcgatgtggggtctgtgaaccctgccagcagaagaccaactgtggtgaatgcacttactgcaagaacagaaagaacagccatcagatctgtaagaaaagaaaatgtgaggagctgaaaaagaaaccatctgttgttgtgcctctggaggttataaaggaaaacaagaggccccagagggaaaagaagcccaaagttttaaaggcagattttgacaacaaaccagtaaatggccccaagtcagaatccatggactacagtagatgtggtcatggggaagaacaaaaattggaattgaacccacatactgttgaaaatgtaactaaaaatgaagacagcatgacaggcatcgaggtggagaagtggacacaaaacaagaaatcacagttaactgatcacgtgaaaggagattttagtgctaatgtcccagaagctgaaaaatcgaaaaactctgaagttgacaagaaacgaaccaaatctccaaaattgtttgtacaaaccgtaagaaatggcattaaacatgtacactgtttaccagctgaaacaaatgtttcatttaaaaaattcaatattgaagaattcggcaagacattggaaaacaattcttataaattcctaaaagacactgcaaaccataaaaacgctatgagctctgttgctactgatatgagttgtgatcatctcaaggggagaagtaacgttttagtattccagcagcctggctttaactgcagttccattccacattcttcacactccatcataaatcatcatgctagtatacacaatgaaggtgatcaaccaaaaactcctgagaatataccaagtaaagaaccaaaagatggatctcccgttcaaccaagtctcttatcgttaatgaaagataggagattaacattggagcaagtggtagccatagaggccctgactcaactctcagaagccccatcagagaattcctccccatcaaagtcagagaaggatgaggaatcagagcagagaacagccagtttgcttaatagctgcaaagctatcctctacactgtaagaaaagacctccaagacccaaacttacagggagagccaccaaaacttaatcactgtccatctttggaaaaacaaagttcatgcaacacggtggttttcaatgggcaaactactaccctttccaactcacatatcaactcagctactaaccaagcatccacaaagtcacatgaatattcaaaagtcacaaattcattatctctttttataccaaaatcaaattcatccaagattgacaccaataaaagtattgctcaagggataattactcttgacaattgttccaatgatttgcatcagttgccaccaagaaataatgaagtggagtattgcaaccagttactggacagcagcaaaaaattggactcagatgatctatcatgtcaggatgcaacccatacccaaattgaggaagatgttgcaacacagttgacacaacttgcttcgataattaagatcaattatataaaaccagaggacaaaaaagttgaaagtacaccaacaagccttgtcacatgtaatgtacagcaaaaatacaatcaggagaagggcacaatacaacagaaaccaccttcaagtgtacacaataatcatggttcatcattaacaaaacaaaagaacccaacccagaaaaagacaaaatccaccccatcaagagatcggcggaaaaagaagcccacagttgtaagttatcaagaaaatgatcggcagaagtgggaaaagttgtcctatatgtatggcacaatatgcgacatttggatagcatcgaaatttcaaaattttgggcaattttgtccacatgattttcctactgtatttgggaaaatttcttcctcgaccaaaatatggaaaccactggctcaaacgaggtccattatgcaacccaaaacagtatttccaccactcactcagataaaattacagagatatcctgaatcagcagaggaaaaggtgaaggttgaaccattggattcactcagcttatttcatcttaaaacggaatccaacgggaaggcattcactgataaagcttataattctcaggtacagttaacggtgaatgccaatcagaaagcccatcctttgacccagccctcctctccacctaaccagtgtgctaacgtgatggcaggcgatgaccaaatacggtttcagcaggttgttaaggagcaactcatgcatcagagactgccaacattgcctggtatctctcatgaaacacccttaccggagtcagcactaactctcaggaatgtaaatgtagtgtgttcaggtggaattacagtggtttctaccaaaagtgaagaggaagtctgttcatccagttttggaacatcagaattttccacagtggacagtgcacagaaaaattttaatgattatgccatgaacttctttactaaccctacaaaaaacctagtgtctataactaaagattctgaactgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgtcccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgcagctgctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtctgaaggcttttctccccctcttaatgcctttgctagtgcagtgtattttttcaaggtgctgttaaaagaaagtcatgttgtcgtttactatcttcatctcacccatttcaagtctgaggtaaaaaaataataatgataacaaaacggggtgggtattcttaactgtgactatattttgacaattggtagaaggtgcacattttaagcaaaaataaaagttttatagttttaaatacataaagaaatgtttcagttaggcattaaccttgatagaatcactcagtttggtgctttaaattaagtctgtttactatgaaacaagagtcatttttagaggattttaacaggttcatgttctatgatgtaaaatcaagacacacagtgttaactctacacagcttctggtgcttaaccacatccacacagttaaaaataagctgaattattatttcatggtgccattgttccaacatcttccaatcattgctagaaaattggcatattcctttgaaataaacttatgaaatgttttctctcttaaaatatttctcctgtgtaaaataaatcattgttgttagtaatggttggaggctgttcataaattgtaaatatatattttaaaagcactttctatttttaaaagtaacttgaaataatatagtataagaatcctattgtctattgtttgtgcatatttgcatacaagagaaatcatttatccttgctgtgtagagttccatcttgttaactgcagtatgtattctaatcatgtatatggtttgtgttcttttactgtgtcctctcacattcaagtattagcaacttgcagtatataaaatagttagataatgagaagttgttaattatctctaaaattggaattaggaagcatatcaccaatactgattaacattctctttggaactaggtaagagtggtctcttcttattgaacaacctcaatttagtttcatcccacctttctcagtataatccatgagaggtgtttccaaaaggagatgagggaacaggataggtttcagaagagtcaaatgcttctaatgtctcaaggtgataaaatacaaaaactaagtagacagatatttgtactgaagtctgatacagaattagaaaaaaaaaattcttgttgaaatattttgaaaacaaattccctactatcatcacatgcctccccaaccccaagtcaaaaacaagaggaatggtactacaaacatggctttgtccattaagagctaattcatttgtttatcttagcatactagatttgggaaaatgataactcatcttttctgataattgcctatgttctaggtaacaggaaaacaggcattaagtttattttagtcttcccattttcttcctattactttattgactcattttattgcaaaacaaaaaggattacccaaacaacatgtttcgaacaaggagaattttcaatgaaatacttgattctgttaaaatgcagaggtgctataacattcaaagtgtcagattccttgggagtatggaaaacctaatggtgcttctcccttggaaatgccataggaagcccacaaccgctaacacttacaattttggtgcaaaagcaaacagttccagcaggctctctaaagaaaaactcattgtaacttattaaaataatatctggtgcaaagtatctgttttgagcttttgactaatccaagtaaaggaatatgaagggattgtaaaaaacaaaatgtccattgatagaccatcgtgtacaagtagatttctgcttgttgaatatgtaaaatagggtaattcattgacttgttttagtattttgtgtgccttagatttccgttttaagacatgtatatttttgtgagcctaaggtttcttatatacatataagtatataaataagtgattgtttattgcttcagctgcttcaacaagatatttactagtattagactatcaggaatacacccttgcgagattatgttttagattttaggccttagctcccactagaaattatttcttcaccagatttaatggataaagttttatggctctttatgcatccactcatctactcattcttcgagtctacacttattgaatgcctgcaaaatctaagtatcacttttatttttctttggatcaccacctatgacatagtaaacttgaagaataaaaactaccctcagaaatatttttaaaagaagtagcaaattatcttcagtataatccatggtaatgtatgcagtaattcaaattgatctctctctcaataggtttcttaacaatctaaacttgaaacatcaatgttaatttttggaactattgggatttgtgacgcttgttgcagtttaccaaaacaagtatttgaaaatatatagtatcaactgaaatgtttccattccgttgttgtagttaacatcatgaatggacttcttaagctgattaccccactgtgggaaccaaattggattcctactttgttggactctctttcctgattttaacaatttaccatcccattctctgccctgtgattttttttaaaagcttattcaatgttctgcagcattgtgattgtatgctggctacactgcttttagaatgctctttctcatgaagcaaggaaataaatttgtttgaaatgacattttctctcataaaa

SEQ ID NO: 3 - protein, 14 aa, Artificial
  Synthetic Molecule 36 Tip1
  DISULFID (1)..(14)
  MOD_RES (1)..(1): ACETYLATION
YLIYAYYTWWEHSC

SEQ ID NO: 4 - protein, 13 aa, Artificial
  Synthetic Molecule 37 Tip2
  MOD_RES (1)..(1): ACETYLATION
  DISULFID (1)..(13)
YWYLPSYRVPWFC

SEQ ID NO: 5 - protein, 17 aa, Artificial
  Synthetic Molecule 38 Tip3m15L
  MOD_RES (1)..(1): ACETYLATION
  DISULFID (1)..(17)
YYRVKTYYQITYVYLSC

(19) United States
(12) Patent Application Publication
(10) Pub. No.: US 2024/0011094 A1
(43) Pub. Date: Jan. 11, 2024

(54) METHODS FOR DIAGNOSIS AND TREATING POLYCYSTIC OVARY SYNDROME (PCOS)

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); CENTRE HOSPITALIER REGIONAL UNIVERSITAIRE DE LILLE, Lille (FR); UNIVERSITÉ DE LILLE, Lille (FR); UNIVERSITÉ DE STRASBOURG, Strasbourg (FR)

(72) Inventors: Paolo GIACOBINI, Lille Cedex (FR); Vincent PREVOT, Lille Cedex (FR); Anne-Laurence BOUTILLIER, Strasbourg (FR); Nour El Houda MIMOUNI, Lille Cedex (FR); Isabel PAIVA DE CASTRO, Strasbourg (FR)

(21) Appl. No.: 18/035,409

(22) PCT Filed: Nov. 5, 2021
(86) PCT No.: PCT/EP2021/080741

§ 371 Date: May 4, 2023

(30) Foreign Application Priority Data
Nov. 6, 2020 (EP) 20306346.6

(51) Int. Cl. C12Q 1/6883

(52) U.S. Cl. CPC C12Q 1/6883; C12Q 2600/154; C12Q 2600/158; C12Q 2600/106

(57) ABSTRACT

In the present invention, inventors provide compelling evidence showing that PCOS neuroendocrine reproductive and metabolic dysfunctions are transmitted in PAMH mice for at least three generations. Inventors employed genome-wide methylated DNA immunoprecipitation (MeDIP) analysis to characterize methylated genes in ovaries from control and PAMH mice of the third generation, the first unexposed transgenerational offspring, together with transcriptome analysis in these tissues. Inventors identified many genes with altered transcriptome expression in ovarian tissues of PCOS-animals and they show that several key molecules associated to the PCOS phenotype are epigenetically regulated through DNA hypomethylation. Inventors report that several differentially methylated signatures found in the ovaries of PCOS-like mice are also present in blood samples from women with PCOS and from daughters born to women with PCOS. Accordingly, the present invention relates to methods for diagnosis of the Polycystic Ovary Syndrome (PCOS) through detection of the methylation status of set of gene of the invention in a biological sample obtained from a subject or a patient. The present invention also relates to a method of preventing or treating a Polycystic Ovary Syndrome (PCOS) in a subject in need thereof.

### FIELD OF THE INVENTION

The present invention relates to methods and kits for diagnostic and monitoring the Polycystic Ovary Syndrome (PCOS). More specifically present invention relates to methods for diagnosis of the Polycystic Ovary Syndrome (PCOS) through detection of the methylation status of set of gene of the invention in a biological sample obtained from a subject or a patient. The present invention also relates to a method of preventing or treating a Polycystic Ovary Syndrome (PCOS) in a subject in need thereof

### BACKGROUND OF THE INVENTION

Polycystic Ovary Syndrome (PCOS) is the main cause of female infertility, affecting 6-20% of women of reproductive age worldwide (Dumesic et al., 2015; March et al., 2010). It is characterized by a wide range of clinical symptoms including hyperandrogenism, oligo-anovulation and, in many cases, metabolic disorders (type 2 diabetes, hypertension and cardiovascular disease) (Boyle and Teede, 2016; Dokras et al., 2017). Despite the detrimental effects on women's health, progress towards a cure for PCOS has been hindered by the absence of a clear mechanistic etiology, lack of prognostic markers and by the complexity of the disease.

Accordingly, there remains an unmet need in the art for specific and rapid diagnostic test for Polycystic Ovary Syndrome (PCOS), reflecting directly the dysfunction of genetic processes.

PCOS has a strong heritable component (Crisosto et al., 2007; Gorsic et al., 2019; Gorsic et al., 2017), as witnessed by the fact that ˜60-70% of daughters born to women with PCOS will eventually manifest the disease (Crisosto et al., 2019; Risal et al., 2019). In line with that, a recent study showed that daughters of mothers with PCOS have a fivefold-increased risk of being diagnosed with PCOS later in life (Risal et al., 2019). It has been suggested that environmental factors, such as excessive androgen (Abbott et al., 2002; Franks and Berga, 2012; Padmanabhan and Veiga-Lopez, 2013; Risal et al., 2019; Walters et al., 2018b), or elevated levels of anti-Müllerian hormone (AMH) exposure (Tata et al., 2018), may be in part responsible for the development of PCOS. Indeed, recent preclinical evidence demonstrated that PCOS may originate in the womb due to the “programming” effect of excessive prenatal AMH exposure (Tata et al., 2018). This animal model, named PAMH, recapitulates all the diagnostic criteria for PCOS in women: hyperandrogenism, oligo-anovulation, altered fertility, together with increased gonadotropin releasing hormone (GnRH) and luteinizing hormone (LH) secretion, which exacerbate the hyperandrogenism in mice (Tata et al., 2018) and humans (Stener-Victorin et al., 2020; Walters et al., 2018b).

Investigation into whether altered intrauterine milieu influences transgenerational susceptibility to PCOS is not feasible in humans due to the difficulty of following existing PCOS cohorts for multiple generations. Therefore, preclinical PCOS models provide translatable alternatives to investigate the mechanisms underlying the etiology of the disease (Stener-Victorin et al., 2020). Consistently, it has been shown that prenatally androgenized (PNA) mice derived from dams exposed to dihydrotestosterone (DHT) during late pregnancy, display PCOS-like phenotypes (Moore et al., 2015; Roland et al., 2010; Sullivan and Moenter, 2004), which are transmitted across three generations (Risal et al., 2019).

Environmental factors have been shown to exert their effects via the induction of epigenetic changes such as DNA methylation and these modifications can lead to increased disease susceptibility later in life. However, there are very few studies focusing on the epigenetic changes associated with PCOS development, with only a handful of genome-wide studies conducted so far (Makrinou et al., 2020; Shen et al., 2013; Wang et al., 2014; Xu et al., 2016; Xu et al., 2010; Yu et al., 2015).

### SUMMARY OF THE INVENTION

A first object of the present invention relates to an in vitro method for assessing a subject's risk of having or developing Polycystic Ovary Syndrome (PCOS), comprising the steps of i) determining in a sample obtained from the subject the methylation status of one or more gene selected from a group of gene consisting of TET1, ROBO1, HDC, IGFBPL1, CDKN1A and IRS4 genes, ii) comparing the methylation status determined in step i) with a reference value and iii) concluding when the methylation status of one or more gene selected from a group of gene consisting of TET1, ROBO1, HDC, IGFBPL1, CDKN1A and IRS4 determined at step i) is lower (hypomethylated) compared with the reference value, it is predictive of a high risk of having or developing Polycystic Ovary Syndrome (PCOS).

An additional object of the invention relates to an in vitro method for monitoring a Polycystic Ovary Syndrome (PCOS) comprising the steps of i) determining the methylation status of one or more gene selected from a group of gene consisting of: TET1, ROBO1, HDC, IGFBPL1, CDKN1A and IRS4 in a sample obtained from the subject at a first specific time of the disease, ii) determining the methylation status of one or more gene selected from a group of gene consisting of TET1, ROBO1, HDC, IGFBPL1, CDKN1A and IRS4 in a sample obtained from the subject at a second specific time of the disease, iii) comparing the methylation status determined at step i) with the methylation status determined at step ii) and iv) concluding that the disease has evolved in better manner when the methylation level of one or more gene selected from a group of gene consisting of TET1, ROBO1, HDC, IGFBPL1, CDKN1A and IRS4 determined at step ii) is higher than the methylation status determined at step i).

An additional object of the invention relates to an in vitro method for monitoring the treatment of Polycystic Ovary Syndrome (PCOS) comprising the steps of i) determining the methylation status of one or more gene selected from a group of gene consisting of TET1, ROBO1, HDC, IGFBPL1, CDKN1A and IRS4 in a sample obtained from the subject before the treatment, ii) determining the methylation status of one or more gene selected from a group of gene consisting of TET1, ROBO1, HDC, IGFBPL1, CDKN1A and IRS4 in a sample obtained from the subject after the treatment”, iii) comparing the level determined at step i) with the level determined at step ii) and iv) concluding that the treatment is efficient when the level determined at step ii) is higher than the level determined at step i).

In a particular embodiment, the sample obtained from the subject, is a blood sample.

Another object of the invention relates to a methylating agent for use in the prevention or the treatment of a Polycystic Ovary Syndrome (PCOS) in a subject in need thereof.

Another object of the invention relates to a TET1 inhibitor for use in the prevention or the treatment of a Polycystic Ovary Syndrome (PCOS) in a subject in need thereof.

### DETAILED DESCRIPTION OF THE INVENTION

To gain further insight on PCOS pathogenesis, inventors provide compelling evidence showing that PCOS neuroendocrine reproductive and metabolic dysfunctions are transmitted in PAMH mice for at least three generations. Inventors employed genome-wide methylated DNA immunoprecipitation (MeDIP) analysis to characterize methylated genes in ovaries from control and PAMH mice of the third generation, the first unexposed transgenerational offspring, together with transcriptome analysis in these tissues. Inventors identified many genes with altered transcriptome expression in ovarian tissues of PCOS-animals and they show that several key molecules associated to the PCOS phenotype are epigenetically regulated through DNA hypomethylation. Inventors report that several differentially methylated signatures found in the ovaries of PCOS-like mice are also present in blood samples from women with PCOS and from daughters born to women with PCOS.

These findings show that the transmission of PCOS reproductive and metabolic dysfunctions to multiple generations occurs via altered landscapes of DNA methylation and identify methylome markers as possible diagnostic landmarks and candidates for epigenetic-based therapies These results thus set-up the basis for the development of a rapid functional specific test for PCOS.

Finally, treatment of PAMH F3 female offspring with a methylating pharmacological agent rescued the neuroendocrine and metabolic alterations of PCOS, thus highlighting a roadmap to new avenues for epigenetic therapies of the disease.

Diagnostic Methods According to the Invention

The present invention relates to an in vitro method for assessing a subject's risk of having or developing Polycystic Ovary Syndrome (PCOS), comprising the steps of i) determining in a sample obtained from the subject the methylation status of one or more gene selected from a group of gene consisting of: TET1, ROBO1, HDC, IGFBPL1, CDKN1A and IRS4, ii) comparing the methylation status determined in step i) with a reference value and iii) concluding when the methylation status of one or more gene selected from a group of gene consisting of: TET1, ROBO1, HDC, IGFBPL1, CDKN1A and IRS4 determined at step i) is lower (hypomethylated) compared with the reference value is predictive of a high risk of having or developing Polycystic Ovary Syndrome (PCOS).

In another term, the present invention relates to an in vitro diagnostic method of having or developing Polycystic Ovary Syndrome (PCOS) in a subject, comprising the steps of i) determining in a sample obtained from the subject the methylation status of one or more gene selected from a group of gene consisting of: TET1, ROBO1, HDC, IGFBPL1, CDKN1A and IRS4 ii) comparing the methylation status determined in step i) with a reference value and iii) concluding when the methylation status of one or more gene selected from a group of gene consisting of: TET1, ROBO1, HDC, IGFBPL1, CDKN1A and IRS4 determined at step i) is lower (hypomethylated) compared with the reference value is predictive of having or developing Polycystic Ovary Syndrome (PCOS).

In some embodiments, the methods of the present invention are performed in vitro or ex vivo.

In the context of the present invention the “diagnosis” is associated with methylation status of one or more gene selected from a group of gene consisting of: TET1, ROBO1, HDC, IGFBPL1, CDKN1A and IRS4 which in turn may be a risk for developing Polycystic Ovary Syndrome (PCOS) disease.

The term “subject” as used herein refers to a mammalian, such as a rodent (e.g. a mouse or a rat), a feline, a canine, a sheep or a primate. In a preferred embodiment, said subject is a human subject. The subject according to the invention can be a healthy subject (not yet diagnosed) or a subject suffering from a given disease such as Polycystic Ovary Syndrome (PCOS).

As used herein, the term “PCOS” or “Polycystic Ovary Syndrome (PCOS)” refers to a life-threatening cutaneous adverse drug reaction (cADR), characterized by massive epidermal necrosis. Polycystic Ovary Syndrome (PCOS) is the main cause of female infertility, affecting 6-20% of women of reproductive age worldwide (Dumesic et al., 2015; March et al., 2010). It is characterized by a wide range of clinical symptoms including hyperandrogenism, oligo-anovulation and, in many cases, metabolic disorders (type 2 diabetes, hypertension and cardiovascular disease) (Boyle and Teede, 2016; Dokras et al., 2017).

PCOS has a strong heritable component (Crisosto et al., 2007; Gorsic et al., 2019; Gorsic et al., 2017), as witnessed by the fact that ˜60-70% of daughters born to women with PCOS will eventually manifest the disease (Crisosto et al., 2019; Risal et al., 2019). In line with that, a recent study showed that daughters of mothers with PCOS have a fivefold-increased risk of being diagnosed with PCOS later in life (Risal et al., 2019). It has been suggested that environmental factors, such as excessive androgen (Abbott et al., 2002; Franks and Berga, 2012; Padmanabhan and Veiga-Lopez, 2013; Risal et al., 2019; Walters et al., 2018b), or elevated levels of anti-Müllerian hormone (AMH) exposure (Tata et al., 2018), may be in part responsible for the development of PCOS.

In particular embodiments, the subject of the present invention suffers from PCOS and/or have been previously diagnosed (or one its parents) with PCOS.

As used herein, the term “sample” or “biological sample” as used herein refers to any biological sample of a subject and can include, by way of example and not limitation, bodily fluids and/or tissue extracts such as homogenates or solubilized tissue obtained from a subject. Tissue extracts are obtained routinely from tissue biopsy. In a particular embodiment regarding the prognostic method of the critical form of the Polycystic Ovary Syndrome (PCOS) according to the invention, the biological sample is a body fluid sample (such blood) or tissue biopsy (such ovarian sample) of said subject.

In particular embodiments, the fluid sample is a blood sample. The term “blood sample” means a whole blood sample and a plasma sample obtained from a subject (e.g. an individual for which it is interesting to determine the methylation status (or gene expression level) of at least one of the gene of the invention can be identified.

As used herein, the term “TET1”, also known as or “Ten-eleven translocation methylcytosine dioxygenase 1” has its general meaning in the art refers to a member of the TET family of enzymes, that in humans is encoded by the TET1 gene (Gene ID 80312). The protein encoded by this gene is a demethylase that belongs to the TET (ten-eleven translocation) family. Members of the TET protein family play a role in the DNA methylation process and gene activation (NCBI “Entrez Gene: TET1 tet methylcytosine dioxygenase 1”: https://www.ncbi.nlm.nih.gov/gene?cmd=retrieve&dopt=default&rn=1&list_uids=80312). DNA methylation is an epigenetic mechanism that is important for controlling gene expression. TET1 catalyzes the conversion of the modified DNA base 5-methylcytosine (5-mC) to 5-hydroxymethylcytosine (5-hmC) (Tahiliani M, et al (2009). “Science. 324 (5929): 930-5. TET1 produces 5-hmC by oxidation of 5-mC in an iron and alpha-ketoglutarate dependent manner (Ito S, et al (2011). Science. 333 (6047): 1300-3). The conversion of 5-mC to 5-hmC has been proposed as the initial step of active DNA demethylation in mammals and additionally, downgrading TET1 has decreased levels of 5-formylcytosine (5-fC) and 5-carboxylcytosine (5-caC) in both cell cultures and mice ((Ito S, et al (2011) Science. 333 (6047): 1300-3)).

One example of TET1 human amino acid sequence (UniProtKB—Q8NFU7) is provided in SEQ ID NO:1 (NCBI Reference Sequence: NP_085128). One example of nucleotide sequence encoding TET1 is provided in SEQ ID NO:2 (NCBI Reference Sequence: NM_030625).

Of course variant sequences of the TET1 may be used in the context of the present invention (as biomarker or therapeutic target), those including but not limited to functional homologues, paralogues or orthologues, transcript variants of such sequences such as:
TET1 isoform X1: (NCBI Reference Sequence: XM_011540204/XP_011538506)
TET1 isoform X2: (NCBI Reference Sequence: XM_011540205/XP_011538507)
TET1 isoform X3 (NCBI Reference Sequence: XM_017016686/XP_016872175)
TET1 isoform X4 (NCBI Reference Sequence: XM_017016688/XP_016872177 and XM_017016687/XP_016872176.)
TET1 isoform X5 (NCBI Reference Sequence: XM_011540206/XP_011538508.)
TET1 isoform X6 (NCBI Reference Sequence: XM_017016689/XP_016872178 and XM_011540207/XP_011538509)

As used herein, the term “ROBO1” (Roundabout homolog 1) also known as “roundabout guidance receptor 1” has its general meaning in the art refers to a protein that, in humans, is encoded by the ROBO1 gene (gene ID 6091). The protein encoded by ROBO1 is structurally similar to a Drosophila integral membrane protein which is encoded by the Drosophila roundabout gene (a member of the immunoglobulin gene superfamily) and is both an axon guidance receptor and a cell adhesion receptor, known to be involved in the decision by axons to cross the central nervous system midline. Two transcript variants encoding different isoforms have been found for ROBO1 (roundabout homolog 1 isoform a precursor: NM_002941/NP_002932 and roundabout homolog 1 isoform b: NM_133631/NP_598334 see NCBI “Entrez Gene: ROBO1 Roundabout homolog 1”: https://www.ncbi.nlm.nih.gov/gene?cmd=retrieve&dopt=default&rn=1&list_uids=6091 #gene-expression)

The term “HDC” or “Histidine decarboxylase” has its general meaning in the art refers to an enzyme that, in humans, is encoded by the HDC gene (gene ID 3067). This gene encodes a member of the group II decarboxylase family and forms a homodimer that converts L-histidine to histamine in a pyridoxal phosphate dependent manner

(see NCBI “Entrez Gene: Histidine decarboxylase”: https://www.ncbi.nlm.nih.gov/gene?db=gene&Cmd=ShowDetailView&TermToSearch=306 7). In mammals, histamine is an important biogenic amine with regulatory roles in neurotransmission, gastric acid secretion immune response (NCBI “Entrez Gene: Histidine decarboxylase”) and inflammation (Hirasawa N. Int J Mol Sci. 2019 January; 20(2): 376). Histidine decarboxylase is the sole member of the histamine synthesis pathway, producing histamine in a one-step reaction. The enzyme employs a pyridoxal 5′-phosphate (PLP) cofactor, in similarity to many amino acid decarboxylases.

As used herein, the term “IGFBPL1” also knows as Insulin-like growth factor-binding protein 1 (IBP-1) or “placental protein 12” (PP12) has its general meaning in the art refers to a protein that, in humans, is encoded by the IGFBPL1 gene (gene ID 3484). This gene is a member of the insulin-like growth factor binding protein (IGFBP) family and encodes a protein with an IGFBP N-terminal domain and a thyroglobulin type-I domain. The encoded protein, mainly expressed in the liver, circulates in the plasma and binds both insulin-like growth factors (IGFs) I and II, prolonging their half-lives and altering their interaction with cell surface receptors. This protein is important in cell migration and metabolism. Low levels of this protein may be associated with impaired glucose tolerance, vascular disease and hypertension in human patients (NCBI “Entrez Gene: “IGFBP1

insulin-like growth factor binding protein 1” https://www.ncbi.nlm.nih.gov/gene?db=gene&Cmd=ShowDetailView&TermToSearch=348 4).

As used herein, the term “CDKN1A” or “cyclin dependent kinase inhibitor 1A”, also known as “p21Cip1” (alternatively p21Waf1) or “CDK-interacting protein 1” has its general meaning in the art refers to a protein that, in humans, is encoded by the CDKN1A gene (gene ID 1026). CDKN1A is a cyclin-dependent kinase inhibitor (CKI) that is capable of inhibiting all cyclin/CDK complexes (Xiong Y, et al. (1993). Nature. 366 (6456): 701-4) though is primarily associated with inhibition of CDK2 (Tarek; A. et al. (2009). Nature Reviews Cancer. 9 (6): 400-414). CDKN1A represents a major target of p53 activity and thus is associated with linking DNA damage to cell cycle arrest (el-Deiry W S et al (November 1993). Cell. 75 (4): 817-25; Bunz F, et al. (1998). Science. 282 (5393): 1497-1501). The expression of this gene is tightly controlled by the tumor suppressor protein p53, through which this protein mediates the p53-dependent cell cycle G1 phase arrest in response to a variety of stress stimuli. This protein can interact with proliferating cell nuclear antigen, a DNA polymerase accessory factor, and plays a regulatory role in S phase DNA replication and DNA damage repair. This protein was reported to be specifically cleaved by CASP3-like caspases, which thus leads to a dramatic activation of cyclin-dependent kinase2, and may be instrumental in the execution of apoptosis following caspase activation. Mice that lack this gene have the ability to regenerate damaged or missing tissue. Multiple alternatively spliced variants have been found for this gene (NCBI “Entrez Gene: cyclin dependent kinase inhibitor 1A” https://www.ncbi.nlm.nih.gov/gene?db=gene&Cmd=ShowDetailView&TermToSearch=102 6.)

As used herein, the term “IRS4” or “Insulin receptor substrate 4”, also known as “CHNG9” or IRS-4; or “PY160” has its general meaning in the art refers to a protein that, in humans, is encoded by the IRS4 gene (gene ID 8471). IRS4 encodes the insulin receptor substrate 4, a cytoplasmic protein that contains many potential tyrosine and serine/threonine phosphorylation sites. Tyrosine-phosphorylated IRS4 protein has been shown to associate with cytoplasmic signalling molecules that contain SH2 domains. The IRS4 protein is phosphorylated by the insulin receptor tyrosine kinase upon receptor stimulation (NCBI “Entrez Gene: Insulin receptor substrate 4” https://www.ncbi.nlm.nih.gov/sites/entrez?db=gene&Cmd=ShowDetailView&TermToSearch=8471.)

As used herein, the term “methylation status of a gene” has its general meaning in the art refers to the DNA methylation level of a gene.

DNA methylation is a biological process by which methyl groups are added DNA. Methylation can change the activity of a DNA segment without changing the sequence. When located in a gene promoter, DNA methylation typically acts to repress gene transcription. In mammals, DNA methylation is essential for normal development and is associated with a number of key processes including genomic imprinting, X-chromosome inactivation, repression of transposable elements, aging, and carcinogenesis. Two of DNA's four bases, cytosine and adenine, can be methylated. In mammals however, DNA methylation is almost exclusively found in CpG dinucleotides, with the cytosines on both strands being usually methylated. GC- and CpG-rich sequences in DNA are termed CpG islands (Bird A P (1986). “CpG-rich islands and the function of DNA methylation”. Nature. 321 (6067)). CpG islands are usually defined as regions with 1) a length greater than 200 bp, 2) a G+C content greater than 50%, 3) a ratio of observed to expected CpG greater than 0.6 (Gardiner-Garden M, et al. (1987) Journal of Molecular Biology. 196 (2): 261-82). DNA methylation may affect the transcription of genes in two ways. First, the methylation of DNA itself may physically impede the binding of transcriptional proteins to the gene (Choy M K, et al. (2010). BMC Genomics. 11 (1): 519), and second, and likely more important, methylated DNA may be bound by proteins known as methyl-CpG-binding domain proteins (MBDs). MBD proteins then recruit additional proteins to the locus, such as histone deacetylases and other chromatin remodeling proteins that can modify histones, thereby forming compact, inactive chromatin, termed heterochromatin. This link between DNA methylation and chromatin structure is very important. DNA methylation is a powerful transcriptional repressor, at least in CpG dense contexts. Transcriptional repression of protein-coding genes appears essentially limited to very specific classes of genes that need to be silent permanently and in almost all tissues. While DNA methylation does not have the flexibility required for the fine-tuning of gene regulation, its stability is perfect to ensure the permanent silencing of transposable elements (Dahlet T, et al. (June 2020). “Nature Communications. 11 (1): 3153)

Measuring the DNA methylation level of a gene can be performed by a variety of techniques well known in the art.

Methods for extracting chromatin from biological samples and determining the methylation level of gene are well known in the art. Commonly, chromatin isolation procedures comprise lysis of cells after one step of crosslink that will fix proteins that are associated with DNA. After cell lysis, Chromatin is fragmented, immunoprecipitated and DNA is recovered. DNA is then extracted with phenol, precipitated in alcohol, and dissolved in an aqueous solution.

The DNA methylation level can be determined by chromatin IP (see for example Boukarabila H., et al, 2009) ChIP-chip or by ChIP-qPCR or MeDIP assay (see for example the materiel and methods part of Example section).

The DNA methylation level of a gene can also be determined by the following assays
Mass spectrometry is a very sensitive and reliable analytical method to detect DNA methylation. MS, in general, is however not informative about the sequence context of the methylation, thus limited in studying the function of this DNA modification.
Methylation-Specific PCR (MSP), which is based on a chemical reaction of sodium bisulfite with DNA that converts unmethylated cytosines of CpG dinucleotides to uracil or UpG, followed by traditional PCR (Hernindez H G, et al. (2013). BioTechniques. 55 (4): 181-97.
Whole genome bisulfite sequencing, also known as BS-Seq, which is a high-throughput genome-wide analysis of DNA methylation. It is based on the aforementioned sodium bisulfite conversion of genomic DNA, which is then sequenced on a Next-generation sequencing platform. The sequences obtained are then re-aligned to the reference genome to determine the methylation status of CpG dinucleotides based on mismatches resulting from the conversion of unmethylated cytosines into uracil.
Reduced representation bisulfite sequencing, also known as RRBS knows several working protocols. The first RRBS protocol was called RRBS and aims for around 10% of the methylome, a reference genome is needed. Later came more protocols that were able to sequence a smaller portion of the genome and higher sample multiplexing. EpiGBS was the first protocol where you could multiplex 96 samples in one lane of Illumina sequencing and were a reference genome was no longer needed.
The HELP assay, which is based on restriction enzymes' differential ability to recognize and cleave methylated and unmethylated CpG DNA sites.
GLAD-PCR assay, which is based on a new type of enzymes—site-specific methyl-directed DNA endonucleases, which hydrolyze only methylated DNA.
ChIP-on-chip assays, which is based on the ability of commercially prepared antibodies to bind to DNA methylation-associated proteins like MeCP2.
Methylated DNA immunoprecipitation (MeDIP), analogous to chromatin immunoprecipitation, immunoprecipitation is used to isolate methylated DNA fragments for input into DNA detection methods such as DNA microarrays (MeDIP-chip) or DNA sequencing (MeDIP-seq).
Pyrosequencing of bisulfite treated DNA. This is the sequencing of an amplicon made by a normal forward primer but a biotinylated reverse primer to PCR the gene of choice. The Pyrosequencer then analyses the sample by denaturing the DNA and adding one nucleotide at a time to the mix according to a sequence given by the user. If there is a mismatch, it is recorded and the percentage of DNA for which the mismatch is present is noted. This gives the user a percentage of methylation per CpG island.
Molecular break light assay for DNA adenine methyltransferase activity—an assay that relies on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase (Wood R J, et al (August 2007). PLOS ONE. 2(8): e801).
Methyl Sensitive Southern Blotting is similar to the HELP assay, although uses Southern blotting techniques to probe gene-specific differences in methylation using restriction digests. This technique is used to evaluate local methylation near the binding site for the probe.
MethylCpG Binding Proteins (MBPs) and fusion proteins containing just the Methyl Binding Domain (MBD) are used to separate native DNA into methylated and unmethylated fractions. The percentage methylation of individual CpG islands can be determined by quantifying the amount of the target in each fraction.
High Resolution Melt Analysis (HIRM or HRMA), is a post-PCR analytical technique. The target DNA is treated with sodium bisulfite, which chemically converts unmethylated cytosines into uracils, while methylated cytosines are preserved. PCR amplification is then carried out with primers designed to amplify both methylated and unmethylated templates. After this amplification, highly methylated DNA sequences contain a higher number of CpG sites compared to unmethylated templates, which results in a different melting temperature that can be used in quantitative methylation detection (Malentacchi F, et al. (2009). Nucleic Acids Research. 37(12): e86)
Ancient DNA methylation reconstruction, a method to reconstruct high-resolution DNA methylation from ancient DNA samples. The method is based on the natural degradation processes that occur in ancient DNA: with time, methylated cytosines are degraded into thymines, whereas unmethylated cytosines are degraded into uracils.
Methylation Sensitive Single Nucleotide Primer Extension Assay (msSNuPE), which uses internal primers annealing straight 5′ of the nucleotide to be detected. Forat S, et al/(2016 Feb. 1). PLOS ONE. 11 (2): e0147973).
Illumina Methylation Assay measures locus-specific DNA methylation using array hybridization. Bisulfite-treated DNA is hybridized to probes on “BeadChips.” Single-base base extension with labeled probes is used to determine methylation status of target sites (“Infinium Methylation Assay|Interrogate single CpG sites”. www.illumina.com)

According to the invention the “reference value” is the DNA methylation level of gene (selected from a group of gene consisting of TET1, ROBO1, HDC, IGFBPL1, CDKN1A and IRS4) determined in a biological sample of a subject not afflicted by a PCOS. Preferably, said normal level of DNA methylation is assessed in a control sample (e.g., sample from a healthy patient, which is not afflicted by a PCOS) and preferably, the average e histone methylation profile level of said gene in several control samples.

According to the invention, the “reference value” or “cut off value” is determined by considering the distribution of the 5′ Methyl-Cytosine (meC) median values for all patients regarding the methylation status of TET1, ROBO1, HDC, IGFBPL1, CDKN1A and IRS4. For instance, in the present study and with the methylation status of TET1, ROBO1, HDC, IGFBPL1 CDKN1A and/or IRS4 gene assessed by MeDIP-PCR assay. Methylation quantification was calculated from qPCR data and reported as the recovery of starting material: % (meDNA-IP/Total input)=2\hat{ }[(Ct(10% input)−3.32)−Ct(meDNA-IP)]×100%. The analysis revealed that the decrease of the DNA methylation level compared to the group control can be e.g. at least 10%, or at least 20%, more preferably at least 50% even more preferably at least 100% and allowed to effectively discriminate PCOS from/control biological sample (blood sample) and this control biological sample could be used as predetermined reference level for TET1, ROBO1, HDC, IGFBPL1 CDKN1A and/or IRS4. (see FIG. 8, “Example section” of the patent application).

The inventors have found biomarkers (methylation status or gene expression level selected from a group of gene) associated with subject's risk to have or to develop a PCOS and have identified 6 biomarkers which could be used separately or in combination.

The term “biomarker”, as used herein, refers generally to a cytogenetic marker, a molecule, the expression of which in a sample from a patient can be detected by standard methods in the art (as well as those disclosed herein), and is predictive or denotes a condition of the subject from which it was obtained.

In preferred embodiments, a plurality of DNA methylation of gene biomarkers (i.e., one or more than one gene expression level biomarkers) or gene expression level biomarkers (i.e., one or more than one gene expression level biomarkers) is used in the method of diagnosis. In other words, the method of the invention may comprise steps of measuring in the biological sample plurality of DNA methylation of gene biomarkers or of gene expression level biomarkers, between one, two, three; four, five, six gene of DNA methylation status gene biomarkers or of expression level biomarker selected from a group of gene consisting of: TET1, ROBO1, HDC, IGFBPL1, CDKN1A and IRS4 gene present in the biological sample.

In particular embodiments, the method of diagnosis is performed using the six different DNA methylation gene biomarkers or the six different gene expression level biomarkers including the TET1, ROBO1, HDC, IGFBPL1, CDKN1A and IRS4 gene.

In the present invention inventors observed that the hypermethylated regions were mostly localized in intronic and intergenic regions, whereas hypomethylated regions were mostly found into upstream-promoters and TSS (Transcription Start Site), thereby most likely affecting gene expression

As the methylation status of a gene is directly associated with gene expression level, the in vitro method of the invention (diagnostic and monitoring) the determination of the methylation status of one or more gene selected from a group of gene consisting of TET1, ROBO1, HDC, IGFBPL1, CDKN1A and IRS4 can be substituted by the determination of the gene expression level of one or more gene selected from a group of gene consisting of TET1, ROBO1, HDC, IGFBPL1, CDKN1A and IRS4.

Thus, in another aspect, the present invention also provides an in vitro method for assessing a subject's risk of having or developing Polycystic Ovary Syndrome (PCOS), comprising the steps of i) determining in a sample obtained from the subject the level of one or more gene expression level selected from a group of gene consisting of TET1, ROBO1, HDC, IGFBPL1 CDKN1A and IRS4 genes, ii) comparing the level determined in step i) with a reference value and iii) concluding when the level of one or more gene expression level selected from a group of gene consisting of TET1, ROBO1, HDC, IGFBPL1 CDKN1A and IRS4 determined at step i) is higher than the reference value is predictive of a high risk of having or developing Polycystic Ovary Syndrome (PCOS).

Measuring the expression level of a gene can be performed by a variety of techniques well known in the art.

Typically, the expression level of a gene may be determined by determining the quantity of mRNA. Methods for determining the quantity of mRNA are well known in the art. For example, the nucleic acid contained in the samples (e.g., blood, cell or tissue extracted from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence-based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3 (4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT (obtained, for example, from Life Technologies (QuantumDot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as ³H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water-soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be affected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from blood and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1, TBP, HPRT1 and TFRC. TATA-binding protein (TBP) and hypoxanthine phosphoribosyl transferase 1 (HPRT1) were used as reference genes in the present study. This normalization allows the comparison of the expression level in one sample, e.g., a patient sample, to another sample, or between samples from different sources.

The man skilled in the art also understands that the same technique of assessment of the expression level of a gene should of course be used for obtaining the reference value and thereafter for assessment of the expression level of a gene of a patient subjected to the method of the invention.

Said reference control values may be determined in regard to the level of gene expression biomarker present in blood samples taken from one or more healthy subject(s) or in a control population.

In one embodiment, the method according to the present invention comprises the step of comparing said level of PCOS-specific gene expression level biomarkers (“Biomarker1”: TET1 gene and/or “Biomarker2”: ROBO1gene and/or “Biomarker3”: HDC gene and/or “Biomarker4”: IGFBPL1 gene and/or “Biomarker5”: CDKN1A gene and/or “Biomarker6”: IRS4 gene) to a control reference value wherein a high level of PCOS-specific gene expression biomarkers (“Biomarker1”: TET1 gene and/or “Biomarker2”: ROBO1gene and/or “Biomarker3”: HDC gene and/or “Biomarker4”: IGFBPL1 gene and/or “Biomarker5”: CDKN1A gene and/or “Biomarker6”: IRS4 gene) compared to said control reference value is predictive of a high risk to of having or developing PCOS and a low PCOS-specific gene expression biomarkers (“Biomarker1”: TET1 gene and/or “Biomarker2”: ROBO1gene and/or “Biomarker3”: HDC gene and/or “Biomarker4”: IGFBPL1 gene and/or “Biomarker5”: CDKN1A gene and/or “Biomarker6”: IRS4 gene) compared to said control reference value is predictive of a low risk to of having or developing PCOS.

The control reference value may depend on various parameters such as the method used to measure the PCOS-specific gene expression level biomarkers (“Biomarker1”: TET1 gene and/or “Biomarker2”: ROBO1gene and/or “Biomarker3”: HDC gene and/or “Biomarker4”: IGFBPL1 gene and/or “Biomarker5”: CDKN1A gene and/or “Biomarker6”: IRS4 gene) or the gender of the subject.

Control reference values are easily determinable by the one skilled in the art, by using the same techniques as for determining the level of gene expression biomarker in a blood samples previously collected from the patient under testing.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the level of gene expression biomarkers (“Biomarker1”: TET1 gene and/or “Biomarker2”: ROBO1gene and/or “Biomarker3”: HDC gene and/or “Biomarker4”: IGFBPL1 gene and/or “Biomarker5”: CDKN1A gene and/or “Biomarker6”: IRS4 gene) with a defined threshold value. In one embodiment of the present invention, the threshold value is derived from the gene expression level (or ratio, or score) determined in a blood sample derived from one or more subjects who are responders (to the method according to the invention). In one embodiment of the present invention, the threshold value may also be derived from gene expression level (or ratio, or score) determined in a blood sample derived from one or more subjects or who are non-responders. Furthermore, retrospective measurement of the gene expression level (or ratio, or scores) in properly banked historical subject samples may be used in establishing these threshold values.

“Risk” in the context of the present invention, relates to the probability that an event will occur over a specific time period, as in humoral immune response of a subject to a vaccine, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no conversion. Alternative continuous measures, which may be assessed in the context of the present invention, include time to humoral immune response of a subject to a vaccine risk reduction ratios.

“Risk evaluation,” or “evaluation of risk” in the context of the present invention encompasses making a prediction of the probability, odds, or likelihood that an event (humoral immune response of a subject to a vaccine) may occur, the rate of occurrence of the event or conversion from one state to another, i.e., from a “PCOS to non PCOS. Risk evaluation can also comprise prediction of future clinical parameters, traditional laboratory risk factor values, or other indices of “humoral response”, such as cellular population determination in peripheral tissues, in serum or other fluid, either in absolute or relative terms in reference to a previously measured population. The methods of the present invention may be used to make continuous or categorical measurements of the risk of a event (having or developing PCOS), thus diagnosing and defining the risk spectrum of a category of subjects defined as having or developing PCOS. In the categorical scenario, the invention can be used to discriminate between normal and other subject cohorts at higher risk to be having or developing PCOS.

Kit for Performing the Method of the Invention

A further object of the invention relates to kits for performing the methods of the invention, wherein said kits comprise means for measuring the expression level (or methylation status) of one or more genes selected from a group of genes consisting of: TET1, ROBO1, HDC, IGFBPL1 CDKN1A and/or IRS4 gene of the invention in the sample obtained from the patient for used to assess a subject's risk to have or to develop PCOS.

Accordingly, the present invention also relates to a kit of the invention comprising means for determining the methylation status or the expression level of one or more genes selected from a group of genes consisting of TET1, ROBO1, HDC, IGFBPL1 CDKN1A and/or IRS4 gene.

In one embodiment, the present invention relates to a kit for use to assess a subject's risk to have or to develop PCOS, comprising:
at least a means for determining the methylation status of one or more gene selected from a group of gene consisting of TET1, ROBO1, HDC, IGFBPL1 CDKN1A and/or IRS4 gene and
instructions for use.

In a particular embodiment, the kit for use comprising:
amplification primers and/or probe for determining the methylation status of one or more gene selected from a group of gene consisting of TET1, ROBO1, HDC, IGFBPL1 CDKN1A and/or IRS4 gene,
instructions for use.

In another embodiment, the present invention relates to a kit for use to assess a subject's risk to have or to develop PCOS, comprising:
at least a means for determining the expression level of one or more gene selected from a group of gene consisting of TET1, ROBO1, HDC, IGFBPL1 CDKN1A and/or IRS4 gene and
instructions for use.

In a particular embodiment, the kit for use comprising:
amplification primers and/or probe for determining the expression level (or methylation status) of one or more gene selected from a group of gene consisting of TET1, ROBO1, HDC, IGFBPL1 CDKN1A and/or IRS4 gene,
instructions for use.

The kits may include probes, primers macroarrays or microarrays as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol.

Monitoring Methods and Management

An additional object of the invention relates to an in vitro method for monitoring a Polycystic Ovary Syndrome (PCOS) comprising the steps of i) determining the methylation status of one or more gene selected from a group of gene consisting of: TET1, ROBO1, HDC, IGFBPL1, CDKN1A and IRS4 in a sample obtained from the subject at a first specific time of the disease, ii) determining the methylation status of one or more gene selected from a group of gene consisting of TET1, ROBO1, HDC, IGFBPL1, CDKN1A and IRS4 in a sample obtained from the subject at a second specific time of the disease, iii) comparing the methylation status determined at step i) with the methylation status determined at step ii) and iv) concluding that the disease has evolved in better manner when one or more gene selected from a group of gene consisting of TET1, ROBO1, HDC, IGFBPL1, CDKN1A and IRS4 determined at step ii) is higher than the methylation status determined at step i).

An additional object of the invention relates to an in vitro method for monitoring the treatment of Polycystic Ovary Syndrome (PCOS) comprising the steps of i) determining the methylation status of one or more gene selected from a group of gene consisting of TET1, ROBO1, HDC, IGFBPL1, CDKN1A and IRS4 in a sample obtained from the subject before the treatment, ii) determining the methylation status of one or more gene selected from a group of gene consisting of TET1, ROBO1, HDC, IGFBPL1, CDKN1A and IRS4 in a sample obtained from the subject after the treatment”, iii) comparing the level determined at step i) with the level determined at step ii) and iv) concluding that the treatment is efficient when the level determined at step ii) is higher than the level determined at step i).

In particular embodiment, the sample obtained from the subject, is a blood sample.

The increase can be e.g. at least 5%, or at least 10%, or at least 20%, more preferably at least 50% even more preferably at least 100%.

An additional object of the invention relates to an in vitro method for monitoring a Polycystic Ovary Syndrome (PCOS) comprising the steps of i) determining the gene expression level of one or more gene selected from a group of gene consisting of TET1, ROBO1, HDC, IGFBPL1, CDKN1A and IRS4 in a sample obtained from the subject at a first specific time of the disease, ii) determining the gene expression level of one or more gene selected from a group of gene consisting of: TET1, ROBO1, HDC, IGFBPL1, CDKN1A and IRS4 in a sample obtained from the subject at a second specific time of the disease, iii) comparing the gene expression level determined at step i) with the gene expression level determined at step ii) and iv) concluding that the disease has evolved in better manner when one or more gene selected from a group of gene consisting of TET1, ROBO1, HDC, IGFBPL1, CDKN1A and IRS4 determined at step ii) is lower than the gene expression level determined at step i).

An additional object of the invention relates to an in vitro method for monitoring the treatment of Polycystic Ovary Syndrome (PCOS) comprising the steps of i) determining the gene expression level of one or more gene selected from a group of gene consisting of TET1, ROBO1, HDC, IGFBPL1, CDKN1A and IRS4 in a sample obtained from the subject before the treatment, ii) determining the gene expression level of one or more gene selected from a group of gene consisting of: TET1, ROBO1, HDC, IGFBPL1, CDKN1A and IRS4 in a sample obtained from the subject after the treatment”, iii) comparing the level determined at step i) with the level determined at step ii) and iv) concluding that the treatment is efficient when the level determined at step ii) is lower than the level determined at step i).

In particular embodiment, the sample obtained from the subject, is a blood sample, preferably plasma sample.

The decrease can be e.g. at least 5%, or at least 10%, or at least 20%, more preferably at least 50% even more preferably at least 100%.

Therapeutic Method

Methylating Agent

In the present invention, inventors show that treatment of PAMH F3 female offspring with a methylating pharmacological agent (SAM) rescued the neuroendocrine and metabolic alterations of PCOS, thus highlighting a roadmap to new avenues for epigenetic therapies of the disease.

According another object of the invention relates to a methylating agent for use in the prevention or the treatment of a Polycystic Ovary Syndrome (PCOS) in a subject in need thereof.

The term Methylation agent in the context of the present invention means any biological or chemical compound capable of adding 5′ Methyl-Cytosine groups to the otherwise hypomethylated DNA.

In a particular embodiment the methylating agent is S-Adenosyl methionine (SAM),

S-Adenosyl methionine (SAM-e/Cas Number 29908-03-0) is a common cosubstrate involved in methyl group transfers, transsulfuration, and aminopropylation. Although these anabolic reactions occur throughout the body, most SAM-e is produced and consumed in the liver (Cantoni, GL (1952). J Am Chem Soc. 74 (11): 2942-3). More than 40 methyl transfers from SAM-e are known, to various substrates such as nucleic acids, proteins, lipids and secondary metabolites. It is made from adenosine triphosphate (ATP) and methionine by methionine adenosyltransferase. In eukaryotic cells, SAM-e serves as a regulator of a variety of processes including DNA, tRNA, and rRNA methylation; immune response; (Ding Wei; et al (2015). Cell Metabolism. 22 (4): 633-645) amino acid metabolism; transsulfuration; and more. Chemically, it is a sulfonium betaine which serves as a source of electrophilic methyl group or as a source of 5′-deoxyadenosyl radical

SAM has the following structure:

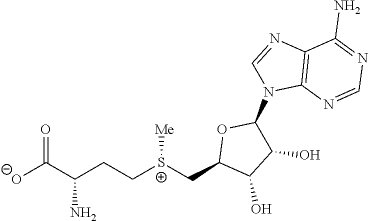 [SMILES: C[S@@+](CC[C@H](N)C(=O)[O-])C[C@H]1O[C@@H](n2cnc3c(N)ncnc32)[C@H](O)[C@@H]1O]

TET1 Inhibitor

TET1 is one of the family members of 5mC dioxygenases, which oxidize 5mC and initiate demethylation. Since the inventors showed: 1) a higher preponderance of hypomethylations in PCOS-like animals and in PCOS women, 2) higher TET1 gene expression in PCOS murine models (see FIG. 7) and significantly hypomethylation in PCOS women (see FIG. 8b), it is likely that the decreased levels of TET1 methylation observed in PCOS women could be at the origin of the preponderance of global DNA hypomethylation characterizing the disease and of the molecular and phenotypic alterations associated with PCOS. These observations reinforce the idea that TET1 should be thus considered a potential target for therapeutic intervention in PCOS.

The inventors show that TET1 is expressed and dysregulated in cells of PCOS subject. TET1 have a potential role in Polycystic Ovary Syndrome (PCOS) pathogenesis.

Accordingly, in an additional aspect the invention relates to a method of preventing or treating a Polycystic Ovary Syndrome (PCOS) in a patient in need thereof comprising administering to the patient a therapeutically effective amount of a TET1 inhibitor.

In a particular embodiment, the invention relates to a TET1 inhibitor for use in the prevention or the treatment of a Polycystic Ovary Syndrome (PCOS) in a subject in need thereof.

In a particular embodiment, the invention relates to a TET1 inhibitor for use in the prevention or the treatment of a Polycystic Ovary Syndrome (PCOS) in a subject in need thereof, wherein the methylation status (or gene expression level) of one or more gene expression level selected from a group of gene consisting of TET1, ROBO1, HDC, IGFBPL1, CDKN1A and IRS4 gene obtained from said patient, have been detected by one of the methods (diagnostic or monitoring) of the invention

In its broadest meaning, the term “treating” or “treatment” refers to reversing, alleviating, inhibiting the progress of Polycystic Ovary Syndrome (PCOS). In particular, “prevention” or “prophylactic treatment” of Polycystic Ovary Syndrome (PCOS) may refer to the administration of the compounds of the present invention that prevent the symptoms of Polycystic Ovary Syndrome (PCOS).

According to the invention, the term “subject” denotes a mammal, such as a rodent, a feline, a canine, or a primate. In some embodiments, the subject is a human. In some embodiments, the subject is a woman. Particularly, the subject denotes a human with a Polycystic Ovary Syndrome (PCOS). As used herein, the term “subject” encompasses the term “patient”.

As used herein, the term “TET1 inhibitor” refers to refers to a natural or synthetic compound that has a biological effect to inhibit the activity or the expression of TET1.

The term “inhibitor” as used herein, refers to an agent that is capable of specifically binding and inhibiting DNA demethylation process and gene activation (such as ROBO1, HDC, IGFBPL1, CDKN1A and IRS4 gene in PCOS) to fully block, as does an inhibitor, or detectably inhibit a response mediated by DNA demethylation process and gene activation (such as ROBO1, HDC, IGFBPL1, CDKN1A and IRS4 gene in PCOS). For example, as used herein the term “TET1 inhibitor” is a natural or synthetic compound which binds and inactivates fully or partially TET1 for initiating or participating to the DNA demethylation process and gene activation (such as ROBO1, HDC, IGFBPL1, CDKN1A and IRS4 gene in PCOS) and further biological processes. In the context of the invention the TET1 inhibitor in particular prevents, decreases or suppresses the DNA demethylation process and gene activation (such as ROBO1, HDC, IGFBPL1, CDKN1A and IRS4 gene in PCOS). The DNA methylation process decrease observed can be by at least about by 20%, 30%, 40%, 50%, 60%, 70%, 80%, or 90%, as compared to the clonal expansion observed in a referenced cell.

TET1 inhibitory activity may be assessed by various known methods. A control TET1 can be exposed to no antibody or antigen binding molecule, an antibody or antigen binding molecule that specifically binds to another antigen, or an anti-TET1 antibody or antigen binding molecule known not to function as an inhibitor, for example as an inhibitor.

In some embodiment, the TET1 inhibitor inhibits the TET1 actions that exacerbate DNA methylation process would be an effective therapeutic option for Polycystic Ovary Syndrome (PCOS) and its consequences.

By “biological activity” of TET1 is meant inducing the DNA demethylation process and gene activation (through the control of expression of ROBO1, HDC, IGFBPL1, CDKN1A and IRS4 gene).

Tests for determining the capacity of a compound to be a TET1 inhibitor are well known to the person skilled in the art. In a preferred embodiment, the inhibitor specifically binds to TET1 (protein or nucleic sequence (DNA or mRNA)) in a sufficient manner to inhibit the biological activity of TET1. Binding to TET1 and inhibition of the biological activity of TET1 may be determined by any competing assays well known in the art. For example, the assay may consist in determining the ability of the agent to be tested as a TET1 inhibitor to bind to TET1. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an inhibitor that “specifically binds to TET1” is intended to refer to an inhibitor that binds to human TET1 polypeptide with a KD of 1 M or less, 100 nM or less, 10 nM or less, or 3 nM or less. Then a competitive assay may be settled to determine the ability of the agent to inhibit biological activity of TET1. The functional assays may be envisaged such as evaluating the ability to: a) inhibit processes associated with DNA demethylation process and/or b) to inhibit gene expression (ie of ROBO1, HDC, IGFBPL1, CDKN1A and IRS4 gene).

The skilled in the art can easily determine whether a TET1 inhibitor neutralizes, blocks, inhibits, abrogates, reduces or interferes with a biological activity of TET1. To check whether the TET1 inhibitor binds to TET1 and/or is able to inhibit TET1 activity (or expression) such as processes associated with inhibition processes associated with DNA demethylation process and/or to inhibit gene expression (of ROBO1, HDC, IGFBPL1, CDKN1A and IRS4 gene) may be performed with each inhibitor. For instance, DNA methylation process may be assessed with aforementioned methods such as ChIP-chip or by ChIP-qPCR or MeDIP assay as described in the Example section (see materiel and method), and gene expression assay can be measured by the aforementioned methods by determining the quantity of mRNA, mRNA is then detected by hybridization (e.g., Northern blot analysis, in situ hybridization, RNAseq) and/or amplification (e.g., RT-PCR).

In a particular embodiment, a TET1 inhibitor according to the invention can be a molecule selected from a peptide, a peptide mimetic, a small organic molecule, an antibody, an aptamer, a polynucleotide (inhibitor of TET1 gene expression) and a compound comprising such a molecule or a combination thereof.

More particularly, the TET1 inhibitor according to the invention is:
1) an inhibitor of TET1 activity (such as, antibody, peptide, aptamer, small organic molecule)
   and/or
2) an inhibitor of TET1 gene expression (such as antisense oligonucleotide, nuclease,)

Antibody or an Antigen-Binding Molecule

The TET1 inhibitor can be an antibody or an antigen-binding molecule. In an embodiment, the antibody specifically recognize/bind TET1 (e.g. TET1 of SEQ ID NO:1) or an epitope thereof involved in a) inhibit processes associated with DNA demethylation and/or b) to inhibit gene expression (ie of ROBO1, HDC, IGFBPL1, CDKN1A and IRS4 gene). In another preferred embodiment, the antibody is a monoclonal antibody.

In a particular embodiment, the TET1 inhibitors may consist in an antibody (the term including antibody fragment or portion) directed against the TET1, that inhibit processes associated with DNA demethylation process in such a way that said antibody impairs the gene expression (ie of ROBO1, HDC, IGFBPL1, CDKN1A and IRS4 gene) (“neutralizing antibody”).

Then, for this invention, neutralizing antibody of TET1 are selected as above described for their capacity to (i) bind to TET1 (protein) and/or ii) inhibit processes associated with DNA demethylation and/or iii) inhibit gene expression (ie of ROBO1, HDC, IGFBPL1, CDKN1A and IRS4 gene).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of TET1. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant TET1 may be provided by expression with recombinant cell lines or bacteria. Recombinant form of TET1 may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

As the TET1 is an intracellular target, the antibody of the invention acting as an activity inhibitor could be an antibody fragment without Fc fragment.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

The skilled artisan can use routine technologies to use the antigen-binding sequences of these antibodies (e.g., the CDRs) and generate humanized antibodies for treatment of cancer disease as disclosed herein.

Peptide Molecule

As indicated previously the TET1 inhibitor can also be a peptide or peptide molecule comprising amino acid residues. As used herein the term “amino acid residue” refers to any natural/standard and non-natural/non-standard amino acid residue in (L) or (D) configuration, and includes alpha or alpha-disubstituted amino acids. It refers to isoleucine, leucine, lysine, methionine, phenylalanine, threonine, tryptophan, valine, arginine, alanine, asparagine, aspartic acid, cysteine, glutamic acid, glutamine, glycine, histidine, proline, serine, tyrosine. It also includes beta-alanine, 3-amino-propionic acid, 2,3-diamino propionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, cyclopentylalanine, cyclobutylalanine, cyclopropylalanine, cyclohexylglycine, cyclopentylglycine, cyclobutylglycine, cyclopropylglycine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (hArg), N-acetyl lysine, 2-amino butyric acid, 2-amino butyric acid, 2,4,-diaminobutyric acid (D- or L-), p-aminophenylalanine, N-methylvaline, selenocysteine, homocysteine, homoserine (HoSer), cysteic acid, epsilon-amino hexanoic acid, delta-amino valeric acid, or 2,3-diaminobutyric acid (D- or L-), etc. These amino acids are well known in the art of biochemistry/peptide chemistry.

Example of peptide used as a TET1 inhibitor for use in the context of the present invention can be selected from specific peptides such as.

TET1 inhibitors based on a scaffold of thioether macrocyclic peptides, which have been discovered by the random nonstandard peptide integrated discovery as described in Nishio K et al “Thioether Macrocyclic Peptides Selected against TET1 Compact Catalytic Domain Inhibit TET1 Catalytic Activity” ChemBioChem 2018, 19, 979-985; The affinity-based selection was performed against the TET1 compact catalytic domain (TET1CCD) to yield thioether macrocyclic peptides. These peptides exhibited inhibitory activity of the TET1 catalyticdomain (TET1CD), with an IC50 value as low as 1.1 mm. One of the peptides, TiP1, was also able to inhibit TET1CD over TET2CD with tenfold selectivity, although it was likely to target the 20G binding site.

Cyclic peptides 36, 37, 38 described in Belle R. Kawamura A and Arimondo P B. (2019) “Chemical Compounds Targeting DNA Methylation and Hydroxymethylation”. Chemical Epigenetics pp 255-286. Part of the Topics in Medicinal Chemistry book series (TMC, volume 33).

Examples of such cyclic peptide TET1 inhibitors are:
Molecule 36 Tip1 (SEQ ID No 3): IC50 (NgTET1)=1.48 μM, having the following structure:

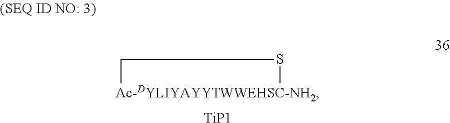

Molecule 37 Tip2 (SEQ ID No 4): IC50 (NgTET1)=1.13 μM, having the following structure:

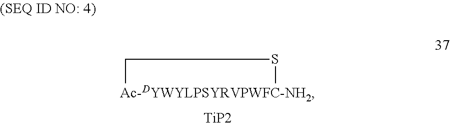

Molecule 38 Tip3m15L: IC50 (NgTET1)=1.44 μM, having the following structure:

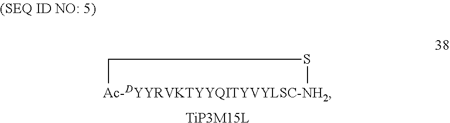

Compounds of the present invention which include peptides may comprise replacement of at least one of the peptide bonds with an isosteric modification. Compounds of the present invention which include peptides may be peptidomimetics. A peptidomimetic is typically characterised by retaining the polarity, three dimensional size and functionality (bioactivity) of its peptide equivalent, but wherein one or more of the peptide bonds/linkages have been replaced, often by proteolytically more stable linkages. Generally, the bond which replaces the amide bond (amide bond surrogate) conserves many or all of the properties of the amide bond, e.g. conformation, steric bulk, electrostatic character, potential for hydrogen bonding, etc. Typical peptide bond replacements include esters, polyamines and derivatives thereof as well as substituted alkanes and alkenes, such as aminomethyl and ketomethylene. For example, the peptide may have one or more peptide linkages replaced by linkages such as —CH2NH—, —CH₂S—, —CH2-CH2-, —CH═CH— (cis or trans), —CH(OH)CH2-, or —COCH2-, —N—NH—, —CH2NHNH—, or peptoid linkages in which the side chain is connected to the nitrogen atom instead of the carbon atom. Such peptidomimetics may have greater chemical stability, enhanced biological/pharmacological properties (e.g., half-life, absorption, potency, efficiency, etc.) and/or reduced antigenicity relative its peptide equivalent.

Aptamer

The TET1 inhibitor can also be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Small Organic Molecule

The TET1 inhibitor can also be a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Examples of small organic molecule are Molecules 3, 28, 29, 30, 31, 32, 33, 34, 35 described in Belle R. Kawamura A and Arimondo P B. (2019) “Chemical Compounds Targeting DNA Methylation and Hydroxymethylation”. Chemical Epigenetics pp 255-286. Part of the Topics in Medicinal Chemistry book series (TMC, volume 33).

Examples of such small organic molecule TET1 inhibitors are:
Molecule 3 20G, (2-oxoglutraric acid): IC50 (NgTET1)=250 μM, having the following structure:

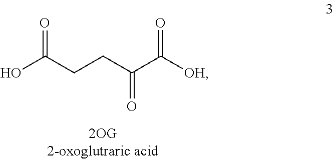

Molecule 28 R-2HG (D-2HG), R-2-hydroxyglutaric acid IC50 {mTet1CD)=4 mM, having the following structure:

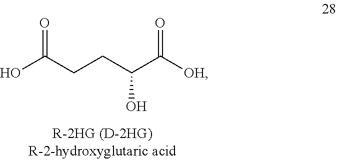

Molecule 29 L-2HG (S-2HG), L-2-hydroxyglutaric acid: IC50 (mTET1)=1 mM, having the following structure:

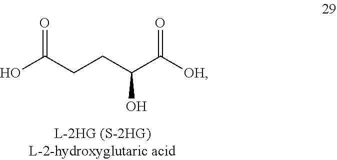

Molecule 30 NOG, N-oxalylglycine: IC50 (NgTET1)=49 μM, having the following structure:

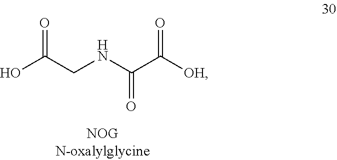

Molecule 31 Succinate: IC50 (mTET1)=540 μM, having the following structure:

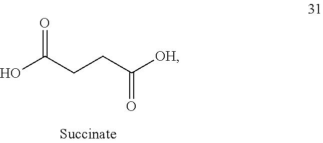

Molecule 32 Fumarate: IC50 (mTET1)=390 μM, having the following structure:

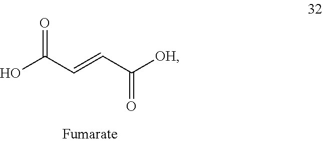

Molecule 33 NgTET1 fluorescent probe: Kd(NgTET1)=250 nM, having the following structure:

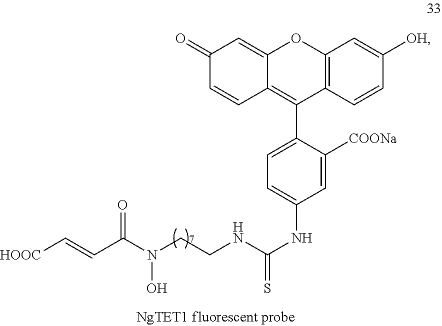

Molecule 34 I OX1, a-hydroxyquinoline: IC50 (hTET1)=1 μM, having the following structure:

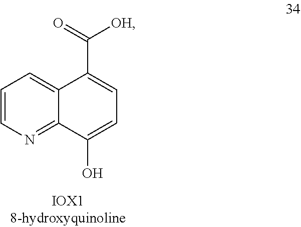 [SMILES: O=C(O)c1ccc(O)c2ncccc12]

Molecule 35 2,4-PDCA, 2,4-pyridinedicarboxylic acid: IC50 (NgTET1)=27 μM, having the following structure:

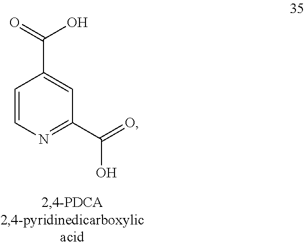 [SMILES: O=C(O)c1ccnc(C(=O)O)c1]

Polynucleotide

The TET1 inhibitor can also be a polynucleotide, typically an inhibitory nucleotide. (Inhibitor of TET1 gene expression). In one embodiment, the inhibitor of TET1 gene expression antibody specifically recognize/bind TET1 nucleic acid sequence (e.g. TET1 of SEQ ID NO:2)

These polynucleotide include short interfering RNA (siRNA), microRNA (miRNA), and synthetic hairpin RNA (shRNA), anti-sense nucleic acids, complementary DNA (cDNA) or guide RNA (gRNA usable in the context of a CRISPR/Cas system). In some embodiments, a siRNA targeting TET1+expression is used. Interference with the function and expression of endogenous genes by double-stranded RNA such as siRNA has been shown in various organisms. See, e.g., Zhao Y et al, “Co-delivery of TET1+siRNA and statin to endothelial cells and macrophages in the atherosclerotic lesions by a dual-targeting core-shell nanoplatform: A dual cell therapy to regress plaques,” Journal of Controlled Release Volume 283, 10 Aug. 2018, p. 241-260; Arjuman A et al “TET1: A potential target for therapy in atherosclerosis; an in vitro study “Int J Biochem Cell Biol. 2017 October; 91(Pt A):65-80. doi: 10.1016. siRNAs can include hairpin loops comprising self-complementary sequences or double stranded sequences. siRNAs typically have fewer than 100 base pairs and can be, e.g., about 30 bps or shorter, and can be made by approaches known in the art, including the use of complementary DNA strands or synthetic approaches. Such double-stranded RNA can be synthesized by in vitro transcription of single-stranded RNA read from both directions of a template and in vitro annealing of sense and antisense RNA strands. Double-stranded RNA targeting TET1 can also be synthesized from a cDNA vector construct in which a TET1 gene (e.g., human TET1 gene) is cloned in opposing orientations separated by an inverted repeat. Following cell transfection, the RNA is transcribed and the complementary strands reanneal. Double-stranded RNA targeting the TET1 gene can be introduced into a cell (e.g., a tumor cell) by transfection of an appropriate construct.

Typically, RNA interference mediated by siRNA, miRNA, or shRNA is mediated at the level of translation; in other words, these interfering RNA molecules prevent translation of the corresponding mRNA molecules and lead to their degradation. It is also possible that RNA interference may also operate at the level of transcription, blocking transcription of the regions of the genome corresponding to these interfering RNA molecules.

The structure and function of these interfering RNA molecules are well known in the art and are described, for example, in R. F. Gesteland et al., eds, “The RNA World” (3rd, Cold Spring Harbor Laboratory Press, Cold Spring Harbor, N.Y., 2006), pp. 535-565, incorporated herein by this reference. For these approaches, cloning into vectors and transfection methods are also well known in the art and are described, for example, in J. Sambrook & D. R. Russell, “Molecular Cloning: A Laboratory Manual” (3rd, Cold Spring Harbor Laboratory Press, Cold Spring Harbor, 2001), incorporated herein by this reference.

In addition to double stranded RNAs, other nucleic acid agents targeting TET1+can also be employed in the practice of the present invention, e.g., antisense nucleic acids. Antisense nucleic acids are DNA or RNA molecules that are complementary to at least a portion of a specific target mRNA molecule. In the cell, the single stranded antisense molecule hybridizes to that mRNA, forming a double stranded molecule. The cell does not translate an mRNA in this double-stranded form. Therefore, antisense nucleic acids interfere with the translation of mRNA into protein, and, thus, with the expression of a gene that is transcribed into that mRNA. Antisense methods have been used to inhibit the expression of many genes in vitro. See, e.g., Li D et al., “Antisense to TET1+inhibits oxidized LDL-mediated upregulation of monocyte chemoattractant protein-1 and monocyte adhesion to human coronary artery endothelial cells “Circulation. 2000 Jun. 27; 101 (25):2889-95. doi: 10.1161; Amati F et al, “TET1+Inhibition in ApoE KO Mice Using a Schizophyllan-based Antisense Oligonucleotide Therapy,” Mol Ther Nucleic Acids. 2012 December; 1(12): e58; incorporated herein by this reference. TET1+polynucleotide sequences from human and many other animals in particular mammals have all been delineated in the art. Based on the known sequences, inhibitory nucleotides (e.g., siRNA, miRNA, or shRNA) targeting TET1+can be readily synthesized using methods well known in the art.

Exemplary siRNAs according to the invention could have up to 29 bps, 25 bps, 22 bps, 21 bps, 20 bps, 15 bps, 10 bps, 5 bps or any integral number of base pairs between these numbers. Tools for designing optimal inhibitory siRNAs include that available from DNAengine Inc. (Seattle, Wash.) and Ambion, Inc. (Austin, Tex).

Examples of siRNAs shRNA, used as TET1 inhibitors are described in Yu T. et al.” Inhibition of Tet1- and Tet2-mediated DNA demethylation promotes immunomodulation of periodontal ligament stem cells” Cell Death & Disease volume 10, Smeriglio P et al; Inhibition of TET1 prevents the development of osteoarthritis and reveals the 5hmC landscape that orchestrates pathogenesis” Science Translational Medicine 15 Apr. 2020:Vol. 12, Issue 539, eaax2332;

Examples of commercial siRNAs shRNA, miRNAs that target human TET1 are also available:
miRNA for TET1 Gene (https://www.genecards.org/cgi-bin/carddisp.pl?gene=TET1): hsa-miR-29b-3p (MIRT004419) hsa-miR-29a-3p (MIRT004420) hsa-miR-21-5p (MIRT030814) hsa-miR-877-5p (MIRT037234) hsa-miR-454-3p (MIRT039236), . . .
RNAi products for human TET1: SR312887 “TET1 Human siRNA Oligo Duplex” (Locus ID 80312) (Browse OriGene Inhibitory RNA Products For TET1); sc-90457 TET1 siRNA and Plasmids shRNA (h) (Santa Cruz Biotechnology (SCBT) for TET1 siRNA/shRNA)

The guide RNA (gRNA) sequences direct a nuclease (i.e. CrispRCas9 protein) to induce a site-specific double strand break (DSB) in the genomic DNA in the target sequence.

Accordingly, Inhibitors of TET1 gene expression for use in the present invention may be based nuclease therapy (like Talen or Crispr).

The term “nuclease” or “endonuclease” means synthetic nucleases consisting of a DNA binding site, a linker, and a cleavage module derived from a restriction endonuclease which are used for gene targeting efforts. The synthetic nucleases according to the invention exhibit increased preference and specificity to bipartite or tripartite DNA target sites comprising DNA binding (i.e. TALEN or CRISPR recognition site(s)) and restriction endonuclease target site while cleaving at off-target sites comprising only the restriction endonuclease target site is prevented.

The guide RNA (gRNA) sequences direct the nuclease (i.e. Cas9 protein) to induce a site-specific double strand break (DSB) in the genomic DNA in the target sequence.

Restriction endonucleases (also called restriction enzymes) as referred to herein in accordance with the present invention are capable of recognizing and cleaving a DNA molecule at a specific DNA cleavage site between predefined nucleotides. In contrast, some endonucleases such as for example Fokl comprise a cleavage domain that cleaves the DNA unspecifically at a certain position regardless of the nucleotides present at this position. Therefore, preferably the specific DNA cleavage site and the DNA recognition site of the restriction endonuclease are identical. Moreover, also preferably the cleavage domain of the chimeric nuclease is derived from a restriction endonuclease with reduced DNA binding and/or reduced catalytic activity when compared to the wildtype restriction endonuclease.

According to the knowledge that restriction endonucleases, particularly type II restriction endonucleases, bind as a homodimer to DNA regularly, the chimeric nucleases as referred to herein may be related to homodimerization of two restriction endonuclease subunits. Preferably, in accordance with the present invention the cleavage modules referred to herein have a reduced capability of forming homodimers in the absence of the DNA recognition site, thereby preventing unspecific DNA binding. Therefore, a functional homodimer is only formed upon recruitment of chimeric nucleases monomers to the specific DNA recognition sites. Preferably, the restriction endonuclease from which the cleavage module of the chimeric nuclease is derived is a type llP restriction endonuclease. The preferably palindromic DNA recognition sites of these restriction endonucleases consist of at least four or up to eight contiguous nucleotides. Preferably, the type llP restriction endonucleases cleave the DNA within the recognition site which occurs rather frequently in the genome, or immediately adjacent thereto, and have no or a reduced star activity. The type llP restriction endonucleases as referred to herein are preferably selected from the group consisting of Pvull, EcoRV, BamHl, Bcnl, BfaSORF1835P, BfiI, Bgll, Bglll, BpuJl, Bse6341, BsoBl, BspD6I, BstYl, Cfr101, Ecl18kl, EcoO109l, EcoRl, EcoRll, EcoRV, EcoR124l, EcoR124ll, HinP11, Hincll, Hindlll, Hpy991, Hpy1881, Mspl, Munl, Mval, Nael, NgoMIV, Notl, OkrAl, Pabl, Pacl, PspGl, Sau3Al, Sdal, Sfil, SgrAl, Thal, VvuYORF266P, Ddel, Eco57l, Haelll, Hhall, Hindll, and Ndel.

Examples of gRNA, used to target TET1 are described in. Choudhury, S R. et al CRISPR-dCas9 mediated TET1 targeting for selective DNA demethylation at BRCA1 promoter. Oncotarget 7, 46545-46556 (2016); Tobias Anton & Sebastian Bultmann (2017) “Site-specific recruitment of epigenetic factors with a modular CRISPR/Cas system,” Nucleus, 8:3, 279-286.

Examples of commercial gRNAs that target human TET1 are also available: Sku: 4651811/TET1 CRISPR Knockout Vector/Virus/Cell Line CRISPR (Applied Biological Materials); CAT #: KN418608TET1 Human Gene Knockout Kit (CRISPR) (Origen), sc-400845 TET1 CRISPR/Cas9 KO Plasmid (h): (Santa Cruz Biotechnology)

Other nuclease for use in the present invention are disclosed in WO 2010/079430, WO2011072246, WO2013045480, Mussolino C, et al (Curr Opin Biotechnol. 2012 October; 23(5):644-50) and Papaioannou I. et al (Expert Opinion on Biological Therapy, March 2012, Vol. 12, No. 3: 329-342) all of which are herein incorporated by reference.

Ribozymes can also function as inhibitors of TET1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of TET1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of TET1 gene expression can be prepared by known methods. These include techniques for chemical synthesis such as, e.g., by solid phase phosphoramadite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing TET1. Preferably, the vector transports the nucleic acid within cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vectors and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in KRIEGLER (A Laboratory Manual,” W.H. Freeman C. O., New York, 1990) and in MURRY (“Methods in Molecular Biology,” vol. 7, Humana Press, Inc., Cliffton, N.J., 1991).

Preferred viruses for certain applications are the adenoviruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, nuclease (i.e. CrispR), siRNA, shRNA or ribozyme nucleic acid sequences are under the control of a heterologous regulatory region, e.g., a heterologous promoter. The promoter may be specific for the ovarian cells or neurons.

Therapeutic Method of a Specific Population

The invention also relates to a method for treating Polycystic Ovary Syndrome (PCOS) with a TET1 inhibitor in a subject having low methylated status of one or more gene selected from a group of gene consisting of: TET1, ROBO1, HDC, IGFBPL1, CDKN1A and IRS4 in a biological sample., wherein the level of methylated status of one or more gene selected from a group of gene consisting of: TET1, ROBO1, HDC, IGFBPL1, CDKN1A and IRS4 obtained from said subject, have been detected by one of method of the invention.

In a preferred embodiment, the biological sample is blood sample or ovarian sample.

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies.

In a particular embodiment, a TET1 inhibitor according to the invention can be a molecule selected from a peptide, a small organic molecule, an antibody, an aptamer, a polynucleotide or a nuclease (inhibitor of TET1 gene expression) and a compound comprising such a molecule or a combination thereof.

Another object of the present invention is a method of treating Polycystic Ovary Syndrome (PCOS) in a subject comprising the steps of:
a) providing a sample from a subject,
b) detecting the methylated status of one or more gene selected from a group of gene consisting of: TET1, ROBO1, HDC, IGFBPL1, CDKN1A and IRS4
c) comparing the level determined at stet b) with a reference value and
if level determined at stet b) is lower than the reference value, treating the subject with a TET1 inhibitor.

The invention will be further illustrated by the following figures and examples. However, these examples and figures should not be interpreted in any way as limiting the scope of the present invention.

### FIGURES

FIG. 1: FIG. 1. Prenatal AMH exposure induces transgenerational transmission of PCOS neuroendocrine traits to multiple generations. a, Schematic illustration of experimental design employed to generate F1, F2, F3 offspring. Gestating mice (FO), prenatally exposed to AMH or PBS from embryonic day 16.5 to 18.5 gave birth to PAMH and control offspring. PAMH F1 females have been mated with unrelated PAMH F1 males to generate PAMH F2 offspring and PAMH F2 females have been mated with unrelated PAMH F2 males to generate PAMH F3 offspring. Control females (CNTR) used throughout the study were the first offspring of gestating mice prenatally treated with PBS. b, Anogenital distance (AGD) measurement over post-natal days (P) 30, 40, 50 and 60 in adult control females (n=14), PAMH F1 (n=13-16), PAMH F2 (n=14), PAMH F3 (n=14). Comparisons between groups were performed using Kruskal Wallis followed by Dunn's multiple comparisons post hoc test: (P30: ****P<0.0001; P40: ****P<0.0001; P50: ****P<0.0001; P60: ****P<0.0001). c, Plasma testosterone concentration in adult females (P60-P90) in diestrus (CNTR F1, n=12; PAMH F1, n=12; PAMH F2, n=14; PAMH F3, n=15; one-way ANOVA: F_{3, 49=39.03}, ****P<0.0001; followed by Tukey's multiple comparison post hoc test.). d, Plasma LH levels in adult (P60-P90) diestrous females (CNTR F1, n=14; PAMH F1, n=11; PAMH F2, n=17; PAMH F3, n=17; one-way ANOVA: F_{3,55}=33.71, ****P<0.0001 followed by Tukey's multiple comparison post hoc test). e, Quantification of the number of corpora lutea (CL) in the ovaries of adult diestrous female mice (CNTR F1, n 8; PAMH F1, n 3; PAMH F3, n=3; one-way ANOVA: F_{2, 11}=15.03, ***P=0.0007; Tukey's multiple comparison post hoc test). Data in c, d, e are represented as mean±s.e.m. *P<0.05; **P<0.005; ***P<0.0005, ****P<0.0001. f, Representative estrous cyclicity of 8 mice/treatment group during 16 consecutive days. M/D: Metestrus/Diestrus phase, P: Proestrus, E: Estrus. g, Quantitative analysis of estrous cyclicity in adult (P60-P90) offspring mice from control and PAMH lineages. Scatter plot representing the percentage (%) of time spent in each estrous cycle respectively in CNTR F1 (n=19), PAMH F1 females (n=19), PAMH F2 females (n=14), PAMH F3 females (n=12). Comparisons between treatment groups were performed using Kruskal-Wallis test followed by Dunn's multiple comparison post hoc test: M/D: ****P<0.0001; P: ****P<0.0001; E: ****P<0.0001. The horizontal line in each scatter plot corresponds to the median value. The vertical line represents the 25^{th}-75^{th} percentile range. h-j, Fertility tests of adult offspring mice (P60). g, Number of pups/litter, h, time to first litter (number of days to first litter after pairing) and i, Fertility index (number of litters per females over 3 months) were quantified per generation and pairing. Statistical analysis was performed using one-way ANOVA and Tukey's multiple comparison post hoc test: Time to first litter, F_{3,25}=27.97, ****P<0.0001; Number of pups/litter, F_{3,25}=43.66 ****P<0.0001; Analysis of fertility index between groups was assessed using Kruskal Wallis test followed by Dunn's multiple comparisons post hoc test: ***P=0.0005. Data h-j are represented as mean±s.e.m. *P<0.05; **P<0.005; ***P<0.0005, ****P<0.0001.

FIG. 2: Prenatal AMH exposure causes a transgenerational increase in body weight, fat mass, and fasting glucose levels in adult female offspring. a, Body composition of CNTR (n=16; 6 months-old), PAMH F1 (n=16; 6 months-old), PAMH F2 (n=11-12; 6 months-old), PAMH F3 (n=16; 6 months-old), presented as body weight (g), percent fat mass normalized to body weight (g) and percent of lean mass. For body weight analysis, statistics was computed with one-way ANOVA (F_{3,56}=23,98 ****P<0.0001) followed by Tukey's multiple comparison post hoc test. Values are represented as the mean±s.e.m. For the analysis of % fat mass and % lean mass, comparisons between groups were performed using Kruskal Wallis followed by Dunn's multiple comparisons post hoc test: (% fat mass: **P=0.0013; % lean mass: **P=0.0033). b, Oral glucose tolerance test (GTT) and insulin tolerance test (ITT; c) in CNTR (n=7; 6 months-old) and PAMH F1 adult female offspring (n=7; 6 months-old). Comparisons between groups at each time point were performed using unpaired Student's t test. Values are represented as the mean±s.e.m. d, Glucose levels upon 12 hours of fasting in CNTR (n=10; 6 months-old), PAMH F1 (n=10; 6 months-old) and PAMH F3 (n=7; 6 months-old) female offspring. Statistics was computed with Kruskal Wallis test followed by Dunn's multiple comparisons post hoc test: (***P=0.0001) Values are represented as the mean±s.e.m. Statistical significance for all analyses were: *P<0.05, **P<0.005, ***P<0.0005, ****P<0.0001.

FIG. 3: RNAseq analysis of ovarian tissue in control and PCOS animals at F3 generation. a, Schematic illustration of the experimental design. b-c, Functional annotation charts using DAVID performed on the differentially regulated genes corresponding to the peaks either decreased in PAMH F3 vs. CNTR (b) or increased in PAMH F3 vs. CNTR (c). Significance is indicated as −log 10 P_value. d-g, Histograms show significantly enrichment in the PAMH F3 ovaries vs CNTR of genes involved in the negative regulation of insulin secretion (d), Follistatin (Fst; e), lipid metabolism (f) and inflammatory response (g). *p_{adj}<0.05; **p_{adj}<0.005; ***p_{adj}<0.0005.

FIG. 4: Top 20 upregulated and downregulated differentially expressed genes and RNA-seq validation. a, b, qPCR validation of 12 differentially expressed genes related to ovarian function, insulin signaling, inflammation, axon guidance, identified by RNA-seq. The mRNA expression levels for 6 upregulated genes (a) and 6 downregulated genes (b) in CNTR (n=5-6) and PAMH F3 ovaries (n=6) dissected from adult females (P60) at dioestrus. Data are presented as mean±s.e.m. P value is determined by unpaired two-tailed Student's t test; n.s, not significant; *, **, P<0.05 and P<0.005, compared with the corresponding controls, respectively. Data were combined from two independent experiments.

FIG. 5 Biological process of hypomethylated and hypermethylated genes in PCOS animals and chromosomal distribution of DNA methylation reads., Representative UCSC Genome Browser views of the Tet Methylcytosine Dioxygenase 1 (Tet1) and Ubiquitin-like, containing PHD and RING finger domains, 1 (Uhrf1) locuses with DNA methylation peaks in ovarian tissues of CNTR vs PAMH F3 mice. Differential methylation analyses revealed that the 5-meC is decreased at the highlighted regions in PAMH F3 mice compared to the CNTR. Tet1: padj=0.018; Uhrf1: padj=0.01/0.02.

FIG. 6 Epigenetic therapy restores PCOS neuroendocrine, reproductive and metabolic traits in PAMH F3 adult females. a, Schematic of experimental design whereby adult (6 months-old) PAMH F3 females have been treated or not with intraperitoneal (i.p.) injections of S-adenosylmethionine (SAM; 50 mg/Kg/day). SAM functions as the primary methyl donor for transmethylation reactions and acts by adding 5′ Methyl-Cytosine groups to the otherwise hypomethylated DNA. b, Representative estrous cyclicity and experimental design. Estrous cyclicity was analyzed in adult CNTR offspring (prenatally PBS-treated; Group 1, n=5, 6 months-old) for 25 days and in PAMH F3 animals during 10 days before treatment. One group of PAMH F3 animals (Group 2; n=5) was then injected daily with PBS and another group of animals (Group 3; n=5) was injected with SAM for 15 days. The Y axis refers to the different stages of the estrous cycle: Metestrus/Dioestrus (M/D), Estrus (E) and Proestrus (P). The X axis represents the time-course of the experiments (days). Tail-blood samples were collected for LH and T measurements at day 10 (dioestrus), before the beginning of the treatment, and trunk-blood was collected at day 25 (dioestrus) at the moment of the sacrifice, corresponding to the end of the treatment period. c, Quantitative analysis of the % of completed estrous cycles in the three experimental groups. The horizontal line in each scatter plot corresponds to the median value. The vertical line represents the 25th-75th percentile range. Comparisons between treatment groups were performed using Kruskal-Wallis test followed by Dunn's post hoc analysis test ***P=0.0002. d, Scatter plot representing the percentage (%) of time spent in each estrous cycle respectively in the three Groups of animals. The horizontal line in each scatter plot corresponds to the median value. The vertical line represents the 25th-75th percentile range. Comparisons between treatment groups were performed using Kruskal-Wallis test followed by Dunn's multiple comparison post hoc test: M/D: ***P=0.0007; E: n.s. P=0.2623; P: **P=0.0026. e, Mean LH levels were measured in dioestrus CNTR F1 mice (n=10), in Group 2 (n=5) and Group 3 (n=5) before the treatment (day 10) and after the treatment (day 25). Statistics was computed with one-way ANOVA (F_{4,25}=21.34, ****P<0.0001) followed by Tukey's multiple comparison post hoc test. f, Mean T levels measured in dioestrus in CNTR F1 mice (n=10) and in Group 2 (n=5) and Group 3 (n=5) before the treatment (day 10) and after the treatment (day 25). Statistics was computed with one-way ANOVA (F_{4,25}=30.17, ****P<0.0001) followed by Tukey's multiple comparison post hoc test. Values in e, f are represented as the mean±s.e.m. g, Body composition in the three experimental groups (n=5 for each group, 6 months-old) presented as body weight (g), percent fat mass normalized to body weight (g) and percent lean mass normalized to body weight (g). h, quantitative analysis of the area of the islets in the three animal groups (CNTR, n=4; PAMH F3, n=5, PAMH F3+SAM, n=5).

For body weight analysis and area of the pancreatic islets, statistics was computed with one-way ANOVA followed by Tukey's multiple comparison post hoc test. Values are represented as the mean±s.e.m. For the analysis of % fat mass and % lean mass, comparisons between groups were performed using Kruskal Wallis followed by Dunn's multiple comparisons post hoc test: (% fat mass F_{4.20}=5.943: **P=0.0026; % lean mass: F_{4.20}=12.09 ****P<0.0001). Statistical significance: *P<0.05, **P<0.005, ***P<0.0005, ****P<0.0001.

FIG. 7 Epigenetic therapy restores expression of genes involved in DNA methylation maintenance and in inflammation in ovarian tissues of PAMH F3 offspring. TaqMan array of the ovaries harvested from CNTR (n=8-9, 6 months-old), PAMH F3 (n=6-9, 6 months-old), PAMH F3-SAM treated (n=5, 6 months-old) offspring at dioestrus. Histograms show on the y-axis relative gene expression (normalized to actin) of Tet1, Uhrf1 Sorbs2, Hdc, Ptgs2, NF-κB. Statistical analysis was performed using Kruskal Wallis followed by Dunn's multiple comparisons post hoc test (Tet1: P=0.1398; Uhrf1: *P=0.0215; Sorbs2: *P=0.0385; Hdc: **P=0.0081; Ptgs2: *P=0.0397; NF-κB: *P 0.0197. Data are presented as mean±s.e.m. *P<0.05; **P<0.005; ***P<0.0005.

FIG. 8 Common epigenetic signatures in human blood samples from women with PCOS. a, Schematic illustration of the experimental design. Genomic DNA was isolated from blood samples of a case-control study comprising two cohorts of women. Group 1: women with and without PCOS (CNTR). Group 2: post-pubertal control daughters born to mothers without PCOS (CNTR-D) and PCOS daughters born to mothers with PCOS (PCOS-D). Methylated DNA immunoprecipitation using antibody against anti-5mC, followed by PCR (MeDIP-PCR) using specific primers against the genes listed in b, c, was performed in the two groups. b, MeDIP-PCR analyses of in CNTR women (n=15) and women with PCOS (n=32), and, c, in daughters from the control group (CNTR-D, n=3) and PCOS daughters of women with PCOS (PCOS-D, n=5). Data are presented as mean±s.e.m. Unpaired two-tailed Mann-Whitney U test, *, **, P<0.05 and P<0.005, compared with the corresponding controls.

### EXAMPLE

Methods:

Study Population: Human Patients

Blood samples have been collected prospectively, from 2003 to 2008, to perform genetic studies, in the Reproductive Medicine Department of Jeanne de Flandre in Lille University Hospital, France. Biological and clinical data about patients were collected at the same time. This study was approved by the Ethics Committee of Lille University Hospital (DRC BT/JR/DS/No 0231 PROM 02-563 CP 03/11). Written informed consent was obtained for all patients. Patients were initially referred to our department for hyperandrogenism (HA) and/or oligo-anovulation and/or infertility. The diagnosis of PCOS was based on the presence of at least 2 out of the 3 following Rotterdam criteria (Rotterdam, 2004), i.e.,: —1) HA (clinical or biological). Clinical HA was defined by the presence of hirsustism (modified Ferriman-Gallwey score over 7 and/or acne located in more than two areas). Hyperandrogenism was defined as a serum TT level >0.7 ng/ml and/or a serum androstenedione level (A) >2.2 ng/ml, as previously reported (Pigny et al., 1997) —2) oligo-anovulation, (i.e. oligomenorrhea or amenorrhea); —3) presence of Polycystic Ovarian Morphology (PCOM) at Ultrasound (U/S), with an ovarian area ≥5.5 cm² and/or a follicle number per ovary ≥12, unilaterally or bilaterally. Women with congenital adrenal hyperplasia, Cushing syndrome, androgen secreting tumor or hyperprolactinemia were excluded. Women with PCOS were asked about familial history and the genetic study was also proposed to their mothers and sisters. The latter were asked about their personal clinical history (age, body mass index, age of first menstruations, cycle length, presence of hirsutism or acne). For sisters who didn't have any contraceptive treatment, hormonal assays were also performed in the follicular phase. Based on these informations, they were classified as PCOS women or control if possible.

Biochemical and hormonal measurement tests were performed in the central biochemistry department of Lille and included: estradiol, LH and FSH, total testosterone, delta4 androstenedione, 17-hydroxyprogesterone, SDHEA, SBP, prolactinemia, fasting glucose, insulinemia and lipid profile. Estradiol, androstenedione, testosterone, LH and FSH were measured by immunoassays as previously described (Pigny et al., 1997). Fasting serum insulin levels were measured in duplicate by an immunoradiometric assay (Bi-Insulin IRMA Pasteur, Bio-Rad, Marnes laCoquette, France) that uses two monoclonal anti-insulin antibodies. Intra and interassay coefficient of variation were <3.8 and <7.5% respectively.

Results are expressed as milli international units per liter. 47 blood samples have been recently analyzed from 32 women with PCOS (18-65 years old) and 15 women without PCOS (22-66 years old). Among the 32 PCOS women, five were born from PCOS mothers (23-30 years old) and among the 15 control women, 3 were confirmed to be born from control mothers (22-36 years old).

All procedures contributing to this work comply with the ethical standards of the relevant national and institutional committees on human experimentation and with the 1975 Declaration of Helsinki, as revised in 2008.

Animals

Timed-pregnant female wild-type C57BL/6J (B6) (Charles River, USA) were group-housed under specific pathogen-free conditions in a temperature-controlled room (21-22° C.) with a 12-h light/dark cycle and ad libitum access to food and water. Standard diet (9.5 mm Pelleted RM3, Special Diets Services, France) was given to all mice during breeding, lactation and growth of young stock. Nutritional profile of the standard diet RM3 is the following: Protein 22.45%, Fat 4.2%, Fiber 4.42%, Ash 8%, Moisture 10%, Nitrogen free extract 50.4%; Calories: 3.6 kcal/gr. Mice were randomly assigned to groups at the time of purchase or weaning to minimize any potential bias. No data sets were excluded from analyses. Animal studies were approved by the Institutional Ethics Committees of Care and Use of Experimental Animals of the University of Lille (France; Ethical protocol number: APAFIS #2617-2015110517317420 v5). All experiments were performed in accordance with the guidelines for animal use specified by the European Council Directive of 22 Sep. 2010 (2010/63/EU). The sample size, sex and age of the animals used is specified in the text and/or figure legends.

Prenatal Anti-Müllerian Hormone (PAMH) Treatment

PAMH animals have been generated as previously described (Tata et al., 2018). Timed-pregnant adult (3-4 months) C57BL6/J (B6) dams were injected daily intraperitoneally (i.p.) from embryonic day (E) 16.5 to 18.5 with 200 μL of a solution containing respectively: 1) 0.01 M phosphate buffered saline (PBS, pH 7.4, prenatal control-treated, CNTR), 2) PBS with 0.12 mgKg⁻¹/d human anti-Müllerian hormone (AMH) (AMHc, R&D Systems, rhMIS 1737-MS-10, prenatal AMH (PAMH)-treated).

Mouse Breeding Scheme and Feeding Paradigm to Generate F1-F3 Offspring

PAMH female offspring (F1) were mated with F1 PAMH unrelated males to generate PAMH F2 offspring, and a subset of PAMH F2 female offspring were mated with PAMH F2 unrelated males to generate PAMH F3 offspring. The remaining F1, F2 and F3 female offspring were subjected to phenotypic testing as described below. Control male or female offspring (CNTR) used in this study were generated by prenatally treating gestating mice with PBS from E16.5 to E18.5 as described above.

The exact number of mice used for each procedure and their sex and age are given in the figure legends and/or text. Details of the number of mice used for (1) phenotypic testing and (2) breeding to generate F1, F2 and F3 offspring in each group are specified in in the figure legends and/or text. To ensure variability within each group, offspring in each generation were randomly allocated for phenotypic testing or breeding.

Assessment of Phenotype, Estrous Cycle, and Fertility

Control F1 and PAMH F1-F3 female offspring were weaned at post-natal day P21 and checked for vaginal opening (VO) and time of first estrus. Anogenital distance (AGD) and body mass (grams, g) were measured at different ages during post-natal development (P30, 35, 40, 50 and 60). At VO and in adulthood (P60), vaginal smears were performed daily for 16 consecutive days (4-cycles) for analysis of age of first estrus and estrous cyclicity. Vaginal cytology was analyzed under an inverted microscope to identify the specific stage of the estrous cycle. The reproductive competency of these animals was determined by pairing the following mice: CNTR F1 females mated with CNTR F1 males, CNTR F1 males mated with PAMH F1-F3 females, PAMH F1-F3 females mated with PAMH F1-F3 males, for a period of 3 months. Unexperienced males and primiparous females, selected from at least three different litters, were used for the 90-days mating protocol test. Number of pups/litter (number of pups), fertility index (number of litters per females over 3 months), and time to first litter (number of days to first litter after pairing) were quantified per treatment and pairing.

Ovarian Histology

Ovaries were collected from 3-month-old dioestrous mice, immersion-fixed in 4% PFA solution and stored at 4° C. Paraffin-embedded ovaries were sectioned at a thickness of 5 pm (histology facility, University of Lille 2, France) and stained with hematoxylin-eosin (Sigma Aldrich, Cat #GHS132, HT1103128). Sections were examined throughout the ovary. Total numbers of corpora lutea (CL) were classified and quantified as previously reported (Caldwell et al., 2017) To avoid repetitive counting, each follicle was only counted in the section where the oocyte's nucleolus was visible. To avoid repetitive counting, CL were counted every 100 μm by comparing the section with the preceding and following sections. CL were characterized by a still present central cavity, filled with blood and follicular fluid remnants or by prominent polyhedral to round luteal cells.

LH and T ELISA Assays

LH levels were determined by a sandwich ELISA, as described previously (Steyn et al., 2013), using the mouse LH-RP reference provided by A. F. Parlow (National Hormone and Pituitary Program, Torrance, CA). Plasma T levels were analyzed using a commercial ELISA (Demeditec Diagnostics, GmnH, DEV9911) (Moore et al., 2015) according to the manufacturers' instructions.

Body Weight and Composition

Whole body fat, fluids, and lean tissue mass were determined by Nuclear Magnetic Resonance (MiniSpec mq 7.5, RMN Analyser, Bruker) according to the manufacturer's recommendations.

Measurement of Fasting Blood Glucose Level

Fasting blood glucose levels were assessed after animals were fasted for 12 h (starting from 8:00 PM). Blood glucose levels were determined in blood samples from the tail vein at 8:00 AM using an automatic glucometer (OneTouch Verio®, Life scan).

Glucose and Insulin Tolerance Tests

For intraperitoneal glucose tolerance test (ipGTT), animals were subjected to an overnight fasting (12 h food withdrawal). For intraperitoneal insulin tolerance test (ipITT), mice were fasted for 4 h. Either glucose (2 g/kg body weight) or human normal insulin (0.75 U/kg body weight) were injected intraperitoneally at 0 (prior to glucose or insulin administration) and blood was collected from the tail vein at different time points (0, 15, 30, 45, 60, 120, 150). Plasma glucose was measured using an automatic glucometer (OneTouch Verio®, Life scan).

RNA Extraction and RT-qPCR

Ovaries tissues were harvest from control F1 and PAMH F3 female mice, frozen ovaries were homogenized using 1 ml of Trizol (ThermoFisher Scientific, Cat #15596026) with a tissue homogenizer and total RNA was isolated using RNeasy Lipid Tissue Mini Kit (Qiagen; Cat #74804) following the manufacturer's instructions. For gene expression analyses, cDNA was synthetized from 1000 ng of total RNA using the High capacity RNA-to-cDNA kit (Applied Biosystems, Cat #4387406) using the manufacturer's recommended cycling conditions. Real-time PCR was carried out on Applied Biosystems 7900HT Fast Real-Time PCR system using exon-boundary-specific TaqMan® Gene Expression Assays (Applied Biosystems) (Table S4). Data were analyzed by using the 2^{-ΔΔCT} method (Livak and Schmittgen, 2001) and normalized to housekeeping genes Beta-actin (ActB) levels. Values are expressed relative to control values, as appropriate, set at 1.

RNA Libraries and Sequencing

RNA-Seq libraries were generated from 600 ng of total RNA using TruSeq Stranded mRNA Library Prep Kit and TruSeq RNA Single Indexes kits A and B (Illumina, San Diego, CA), according to manufacturer's instructions. Briefly, following purification with poly-T oligo attached magnetic beads, the mRNA was fragmented using divalent cations at 94° C. for 2 minutes. The cleaved RNA fragments were copied into first strand cDNA using reverse transcriptase and random primers. Strand specificity was achieved by replacing dTTP with dUTP during second strand cDNA synthesis using DNA Polymerase I and RNase H. Following addition of a single ‘A’ base and subsequent ligation of the adapter on double stranded cDNA fragments, the products were purified and enriched with PCR (30 sec at 98° C.; [10 sec at 98° C., 30 sec at 60° C., 30 sec at 72° C.]×12 cycles; 5 min at 72° C.) to create the cDNA library. Surplus PCR primers were further removed by purification using AMPure XP beads (Beckman-Coulter, Villepinte, France) and the final cDNA libraries were checked for quality and quantified using capillary electrophoresis. Libraries were then single-read sequenced with a length of 50 pb, with 8 samples per lane on an Illumina Hiseq4000 sequencer. Image analysis and base calling were carried out using RTA v.2.7.3 and bcl2fastq v.2.17.1.14. Reads were mapped onto the mm10 assembly of Mus musculus genome using STAR (Dobin et al., 2013) v.2.5.3a. Gene expression was quantified from uniquely aligned reads using HTSeq-count (Anders et al., 2015) v.0.6.1p1 with annotations from Ensembl release 97 and union mode. Data quality was evaluated with RSeQC (Wang et al., 2012). Comparisons of read counts were performed using R 3.5.1 with DESeq2 (Love et al., 2014) v1.22.1 Bioconductor package. More precisely, counts were normalized from the estimated size factors using the median ratio method and a Wald test was used for the statistical test. Unwanted variation was identified with sva (Leek, 2014) and considered in the statistical model. To reduce false positive, p-values were adjusted by IHW method (Ignatiadis et al., 2016).

MeDIP

MeDIP was performed using MagMeDIP kit (Diagenode) according to the manufacturer's instructions. Briefly, frozen mouse ovaries (dissected at dioestrus) were chopped and lysed in 1 mL GenDNA digestion buffer and DNA was extracted using phenol:chloroform:isoamyl alcohol (25:24:1). DNA was quantified using the Qubit™ DNA BR Assay kit. 1.1 ug of DNA was sheared by sonication for six cycles with 30 s ON and 30 s OFF at 4° C. using the Bioruptor Plus sonicator (Diagenode). Immunoprecipitation was performed using an anti-5′-methylcytosine mouse monoclonal antibody (Diagenode; Cat nr: C15200081; Lot nr: RD004; 0.2 ug/immunoprecipitation) or a mouse IgG as a negative control (Diagenode; Cat nr: C15400001; Lot nr: MIG002S; 0.2 ug/immunoprecipitation) and magnetic beads, following MagMeDIP kit settings. One-tenth of the DNA sample was set aside at 4° C. for input. To check the efficiency of the MeDIP experiment, spike-in controls including unmethylated (unDNA) and in vitro methylated DNA (meDNA) from A. thaliana were used. After magnetic beads washes, methylated DNA was isolated using the DNA Isolation Buffer protocol according to the MagMeDIP kit recommendations. DNA concentration was measured using Qubit dsDNA HS Assay Kit (Thermo Fisher). Efficiency of the immunoprecipitation was assessed by performing qPCR using meDNA and unDNA primers.

MeDIP experiments from human blood were carried using the MagMeDIP protocol as described above with some modifications. DNA was extracted from 200 uL of frozen blood using the QIamp DNA blood Mini kit (Qiagen) according to the manufacturer's instructions. RNase A was added prior to cell lysis. DNA was eluted in 100 uL of water. Efficiency of the immunoprecipitation was assessed by performing qPCR for the human TSH2B (methylated region) and GAPDH (unmethylated region) (primers provided in the MagMeDIP kit). Methylation quantification was calculated from qPCR data and reported as the recovery of starting material: % (meDNA-IP/Total input)=2\hat{ }[(Ct(10% input)−3.32)−Ct(meDNA-IP)]×100%.

MeDIP-seq—Libraries Construction and Sequencing

Libraries were prepared using the SMART cDNA Library Construction Kit and sequenced on Illumina Hiseq 4000 sequencer as single-end 50 bp reads following Illumina's instructions. Image analysis and base calling were performed using RTA 2.7.3 and bcl2fastq 2.17.1.14. Adapter dimer reads were removed using Dimer Remover. Data were preprocessed with Cutadapt v1.13 (Martin, 2011) to remove the first 9 nucleotides and to remove sequences with a trailing polyT of at least 10 Ts. Cutadapt was used with the following parameters ‘-u 9 -a T(10) --discard-trimmed’. Reads were mapped to the mouse genome (mm10) using Bowtie v1.0.0 (Langmead et al., 2009) with default parameters except for “-p 3 -m 1 --strata --best”. Methylated regions were detected using MACS v1.4.2 (Zhang et al., 2008) with default parameters except for “-g mm -p le-3”. Regions were then annotated with the closest genes with Homer v4.9.1 annotatePeaks.pl (Heinz et al., 2010) with Ensembl v90 annotations.

All regions found in at least 2 replicates of the same condition were retained for the detection of differentially methylated regions. They were then combined to get the union of all peaks using the tool Bedtools merge v2.26.0 (Quinlan and Hall, 2010). Read counts were normalized across libraries using the method proposed by (Anders and Huber, 2010). Statistical comparisons of interest were performed using the method proposed by (Love et al., 2014) implemented in the DESea2 v1.22.2 Bioconductor library. P-values were adjusted for multiple testing using the (Benjamini, 1995) method. MA plot and Manhattan plots are been generated using custom R scripts.

Methyl Donor S-adenosylmethionine (SAM) Treatment

PAMH F3 female offspring (6 months-old) were cycled for 10 days before treatment, and for additional 15 days during the treatment. CNTR female offspring (6 months-old) were not treated and were cycled for 25 days. Vaginal cytology was analyzed under an inverted microscope to record the specific stage of the estrous cycle. PAMH F3 offspring were injected intraperitoneally (i.p.) daily for 15 days with 200 μL of a solution containing 0.01M phosphate buffered saline (PBS, pH 7.4) or with SAM (50 mg/Kg/day; New England Biolegends, Cat. B9003S). This concentration was chosen based on previous in vivo pharmacological studies using the same drug (Li et al., 2012). Tail-blood samples were collected for LH and T measurements at dioestrus before the beginning of the treatments, at day 10, and at the end of the treatment, at day 25.

Statistical Analyses

All analyses were performed using Prism 8 (Graphpad Software, San Diego, CA) and assessed for normality (Shapiro-Wilk test and/or D'Agostino & Pearson test) and variance, when appropriate. Sample sizes were chosen according to standard practice in the field. The investigators were not blinded to the group allocation during the experiments. However, analyses were performed by two independent investigators in a blinded fashion. For each experiment, replicates are described in the figure legends. No samples were excluded from the analyses.

All comparisons between groups, whose distribution was not normal, were performed using Mann-Whitney U test (comparison between two experimental groups) or Kruskal-Wallis test (comparison between three or more experimental groups) followed by a Dunn's post hoc analysis. The significance level was set at P<0.05. For analyses of populations normally distributed, data were compared using an unpaired two-tailed Student's t-test or a one-way ANOVA for multiple comparisons followed by Tukey's multiple comparison post-hoc test. The number of biologically independent experiments, sample size, P values, age and sex of the animals are all indicated in the main text or figure legends. All experimental data are indicated as mean±s.e.m or as the 25^{th}-75^{th} percentile, line at median. The significance level was set at P<0.05.

Data and Materials Availability

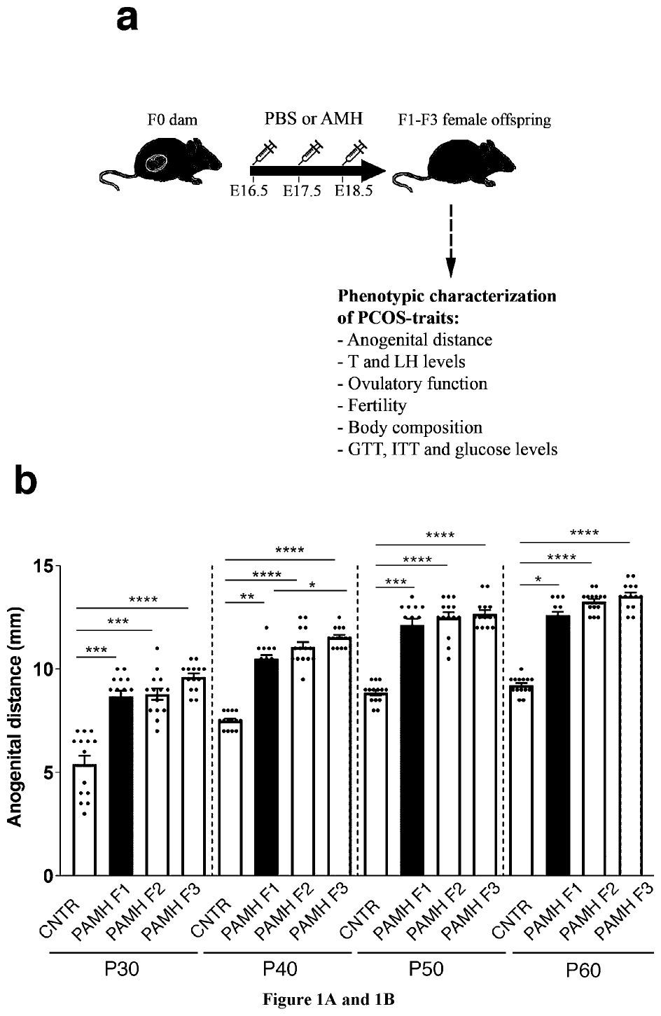

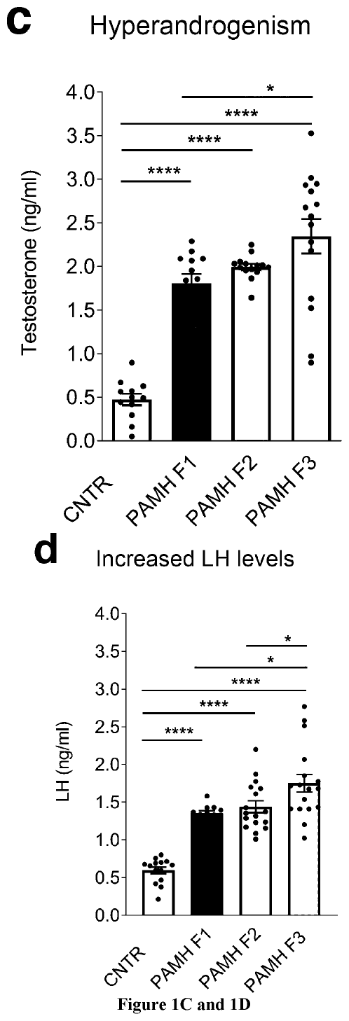

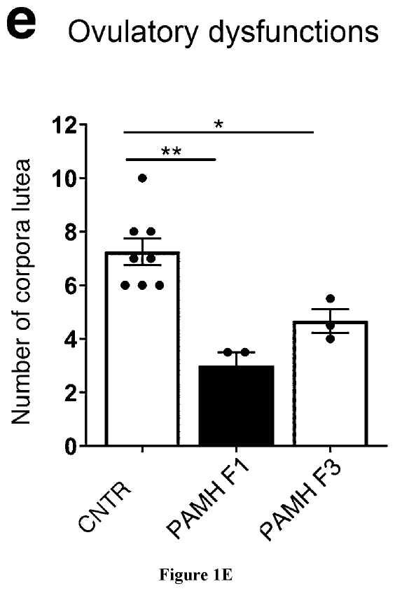

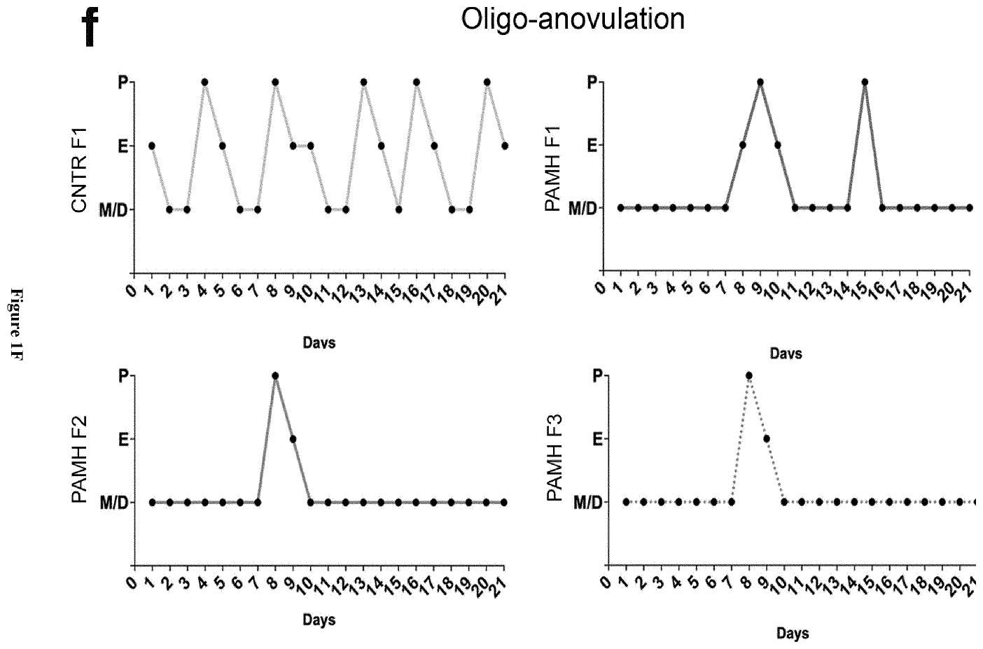

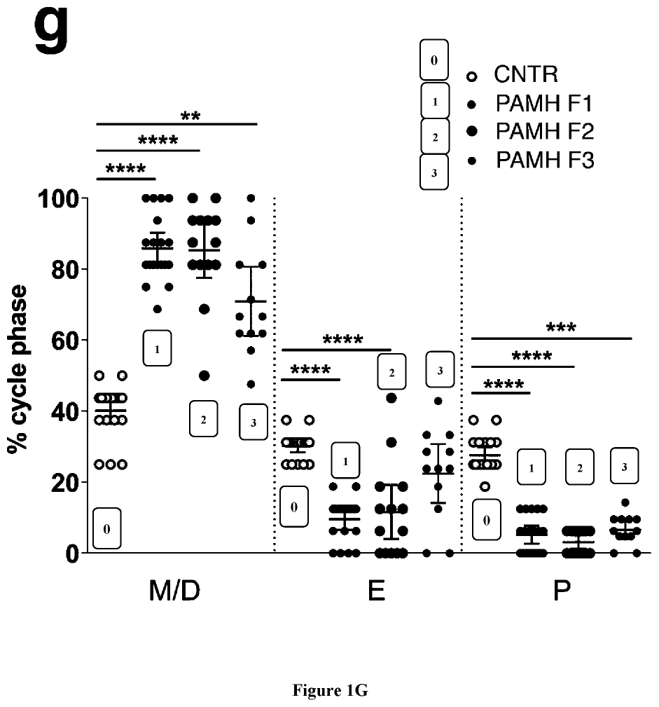

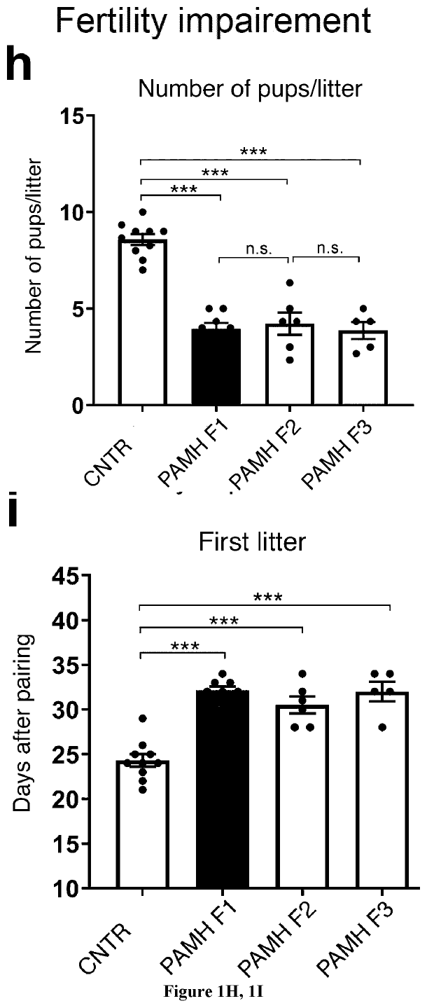

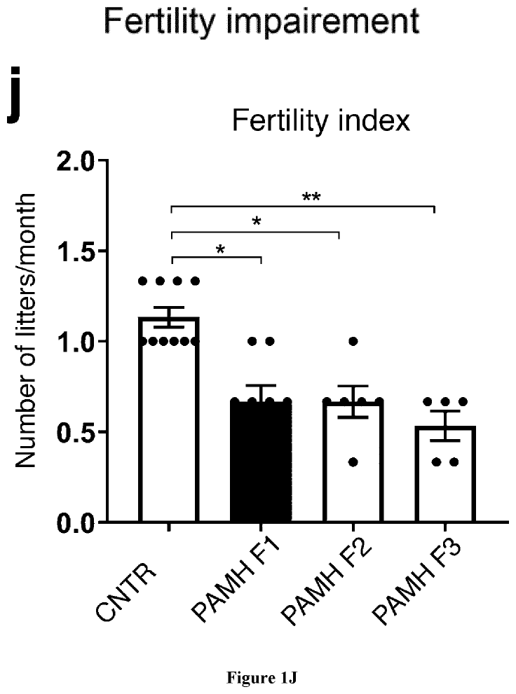

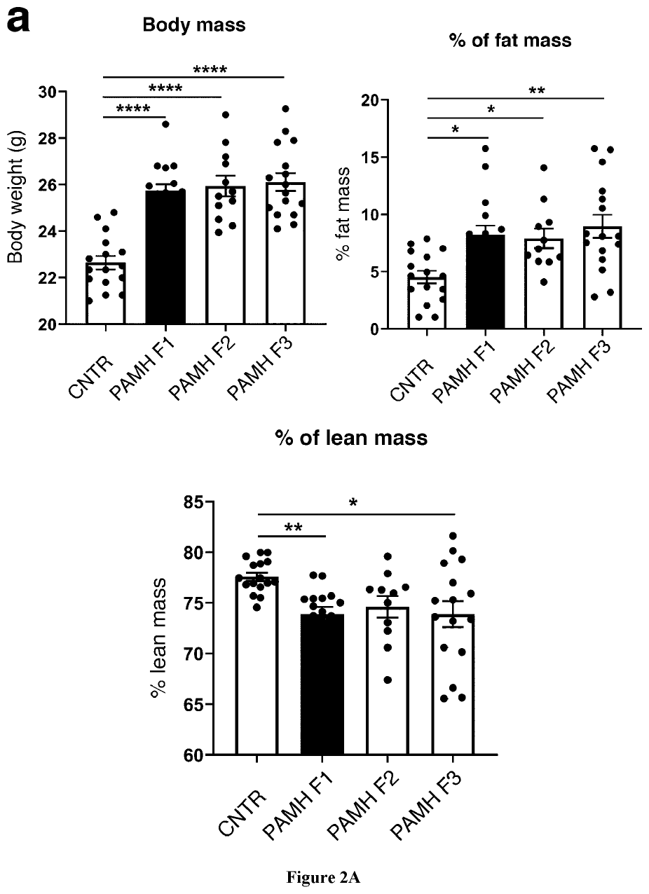

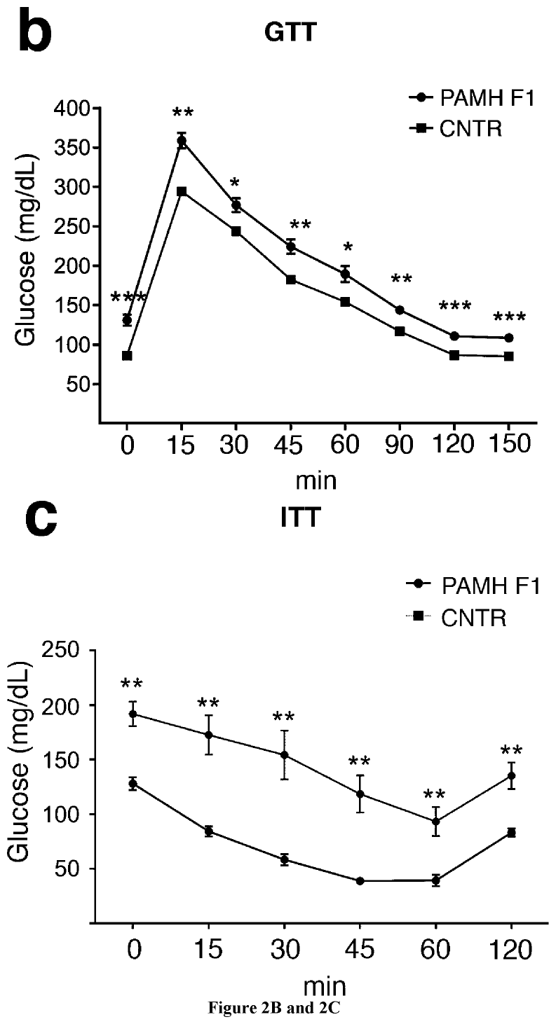

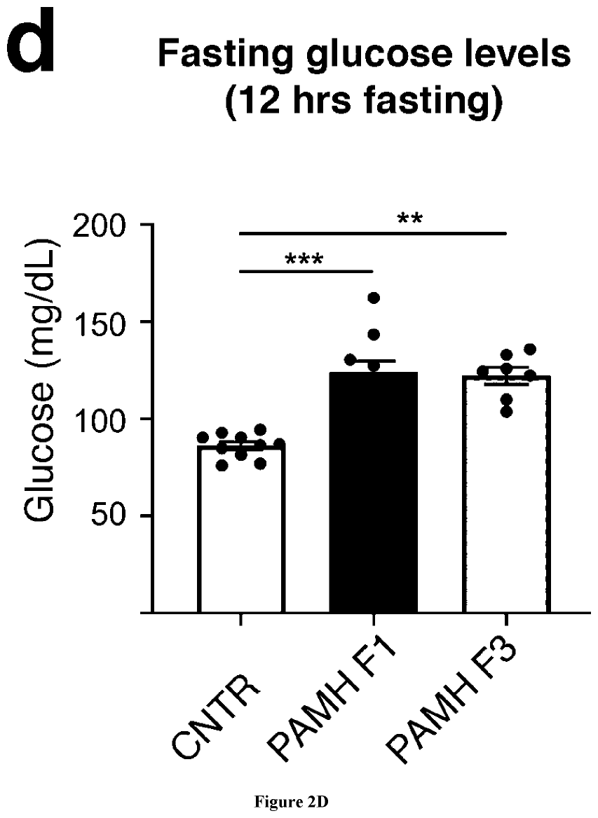

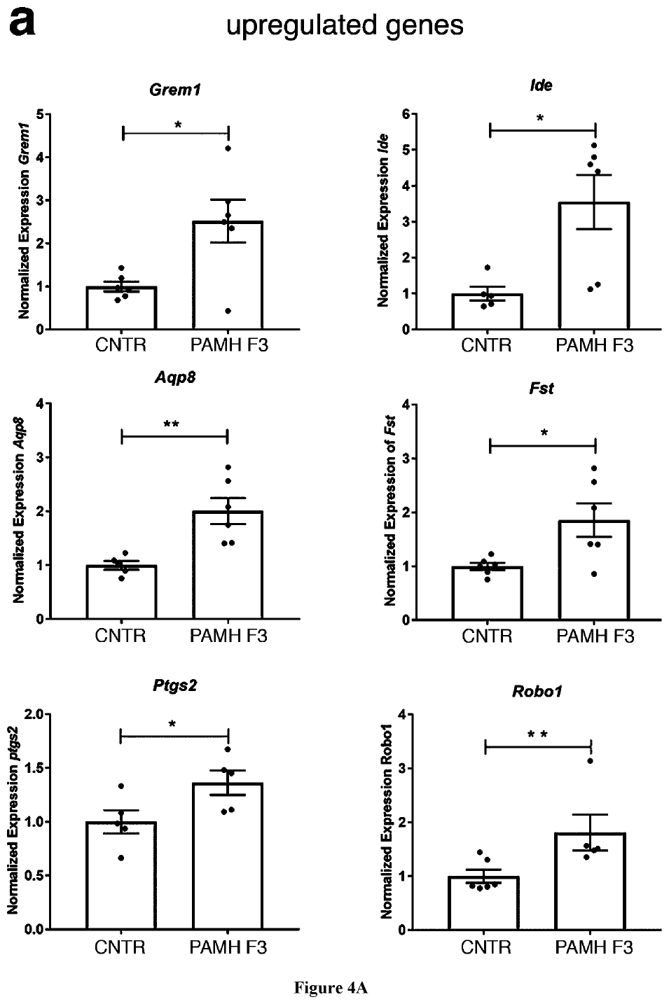

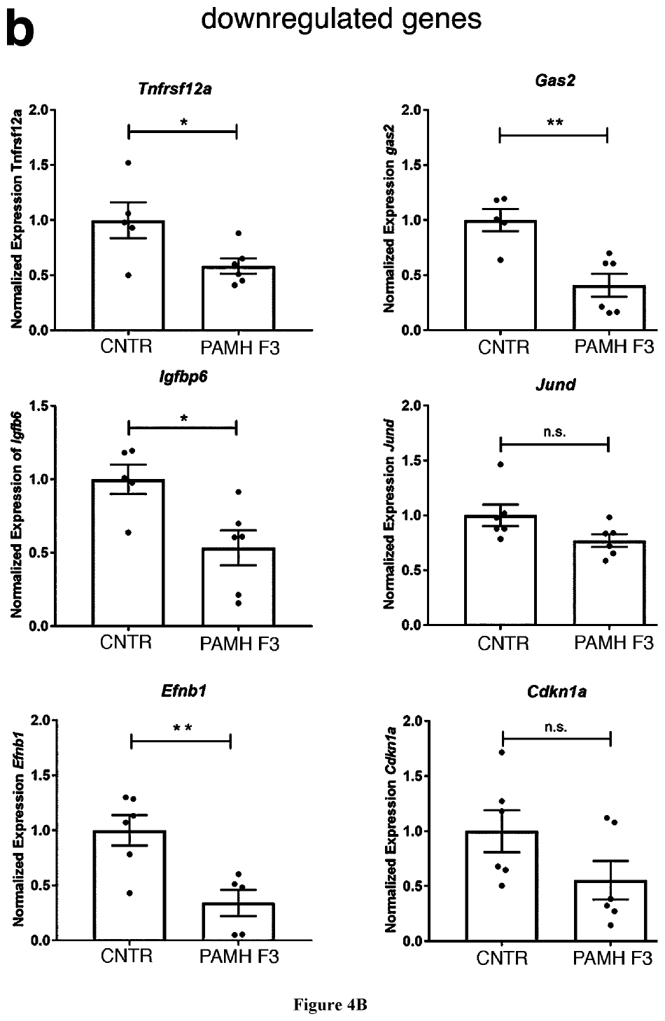

Raw data of Control and PAMH F1-F3 females and of case-control human study are presented in this paper as source data for FIGS. 1-8, Figs. S1, S2, S6 and Extended Data FIGS. 2, 4. All raw RNA-seq and MeDIP-seq data of mouse ovaries from CNTR and PAMH F3 females are available at the Gene Expression Omnibus database via accession number GSE148839.

Results

Prenatal AMH Treatment Drives Transgenerational Transmission of Reproductive and Metabolic PCOS Alterations Across Multiple Generations.

Giving the strong heritability of PCOS and the well documented transmission of the cardinal neuroendocrine features observed in first degree relatives of PCOS women (Sir-Petermann et al., 2012; Sir-Petermann et al., 2002), we sought to test whether female PCOS-like offspring (F1) of gestating mice prenatally exposed to high AMH (F0) (Tata et al., 2018) are susceptible to transfer PCOS-like neuroendocrine reproductive traits to F2 (intergenerational) and to F3 (transgenerational) offspring.

Pregnant dams (FO) were injected intraperitoneally with PBS (CNTR) or with AMH (AMHc, 0.12 mg/Kg/d; prenatal AMH-treated, PAMH) from embryonic day E16.5 to E18.5, to generate CNTR F1 and PAMH F1, respectively. PAMH F1 females were mated with PAMH F1 unrelated males to generate PAMH F2 offspring and F2 female offspring were mated with another group of unrelated males to generate F3 offspring (FIG. 1a). Previous studies have shown that PAMH F1 female offspring manifest all the major criteria of PCOS diagnosis in humans, namely, hyperandrogenism, oligo-anovulation, increased LH levels and fertility impairments (Qi et al., 2019; Tata et al., 2018). We then assessed whether these neuroendocrine reproductive alterations were systematically present in PAMH F2 and F3 offspring. From postnatal day 30 (P30) to P60, F1, F2 and F3 female PAMH lineage exhibited longer anogenital distance than control offspring (FIG. 1b), indicating a higher androgenic impregnation in the PAMH lineage.

PAMH F1-F3 female offspring exhibited delayed vaginal opening and delayed puberty onset (Data not shown).

Subsequently, we uncovered a significant and persistent elevation in both circulating levels of testosterone and LH in adult PAMH F1-F3 females in comparison with the control group (FIG. 1c, d). Ovarian histology of PAMH animals showed comparable abnormalities at F1 and F3 consistent with their anovulatory phenotype, with the presence of fewer post-ovulation corpora lutea as compared to control animals (FIG. 1e). Such ovulatory problems were confirmed by monitoring the estrous cycles of these animals over three weeks and showing that F1, F2 and F3 offspring in the PAMH lineage displayed disrupted estrous cycles with prolonged time in metestrus and dioestrus as compared to the control offspring (Figure if, g). PAMH lineage also showed impaired fertility from F1 to F3, as indicated by fewer pups per litter produced over a 3-month period (FIG. 1h), by a significant delay in their first litter (FIG. 1i) and by fewer litters produced during the 90-days mating protocol (FIG. 1j). Similar ovulatory and fertility defects were detected when PAMH female offspring were mated with control naïve males in a matriline breeding scheme (Data not shown). These data suggest that the reproductive defects of the PAMH lineage (F1-F3) are most likely inherited from the mother.

We then checked whether PAMH F1-F3 female offspring presented PCOS-like metabolic alterations. At P60, the PAMH lineage did not show any difference in body weight as compared with control females (Data not shown). However, at six months of postnatal life, PAMH F1-F3 animals had increased body weight, which was associated with increased fat mass, compared with controls (FIG. 2a). The percentage of free body fluids was comparable between all groups (FIG. 2a), further substantiating that the increased body mass of PAMH mice derive from their increased adiposity. Glucose tolerance and insulin sensitivity were lower in 6 months-old PAMH F1 offspring compared with controls (FIG. 2b, c). Since these defects are reminiscent of type 2 diabetes, we then measured fasting glucose levels, upon 12 hours night fasting conditions, in control and PAMH F1 and F3 female offspring. Fasting glucose levels were significantly increased in PAMH F1 and PAMH F3 animals compared with controls (FIG. 2d), suggesting that these animals are diabetic.

To be considered transgenerational transmission, the inherited traits should be displayed in the third generation (F3), being the first unexposed transgenerational offspring, whereas F1 fetuses and the germ cells of the second generation (F2) are directly exposed to the maternal intrauterine milieu. Since we found that all hormonal, reproductive and metabolic alterations of the F1 offspring are maintained in the third generation, our results show that ancestral exposure to elevated AMH levels during late gestation drives the transgenerational transmission of PCOS traits to multiple generations.

Prenatal AMH Exposure Results in Altered Ovarian Transcriptomic Profiles in the Third-Generation Offspring.

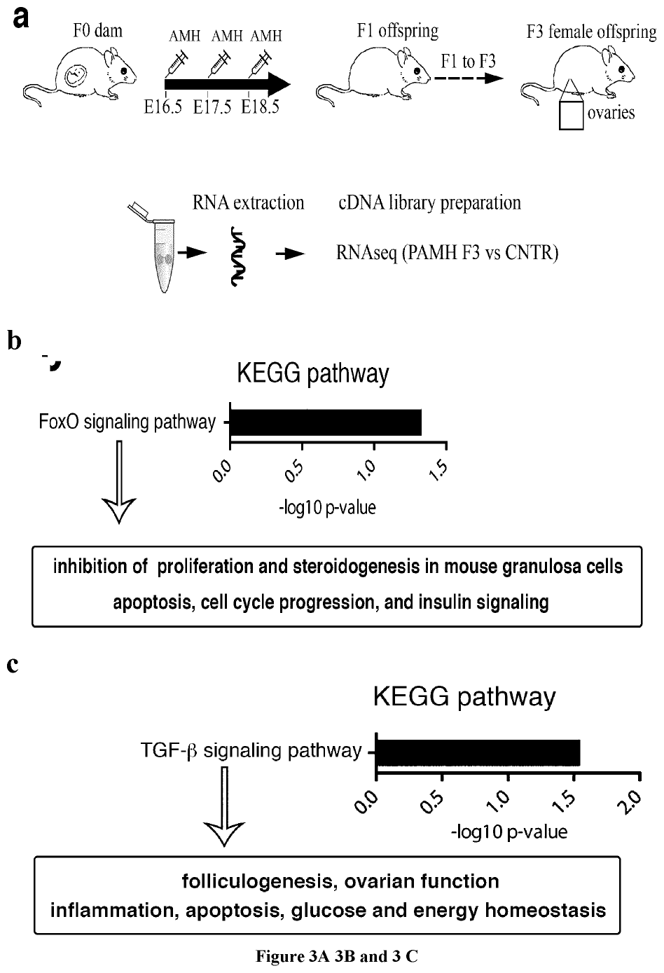

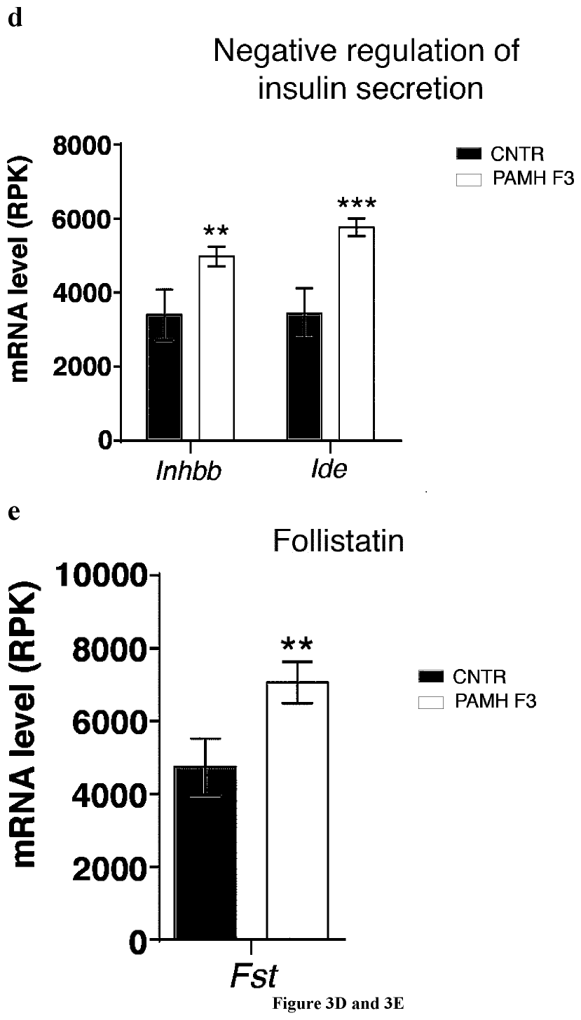

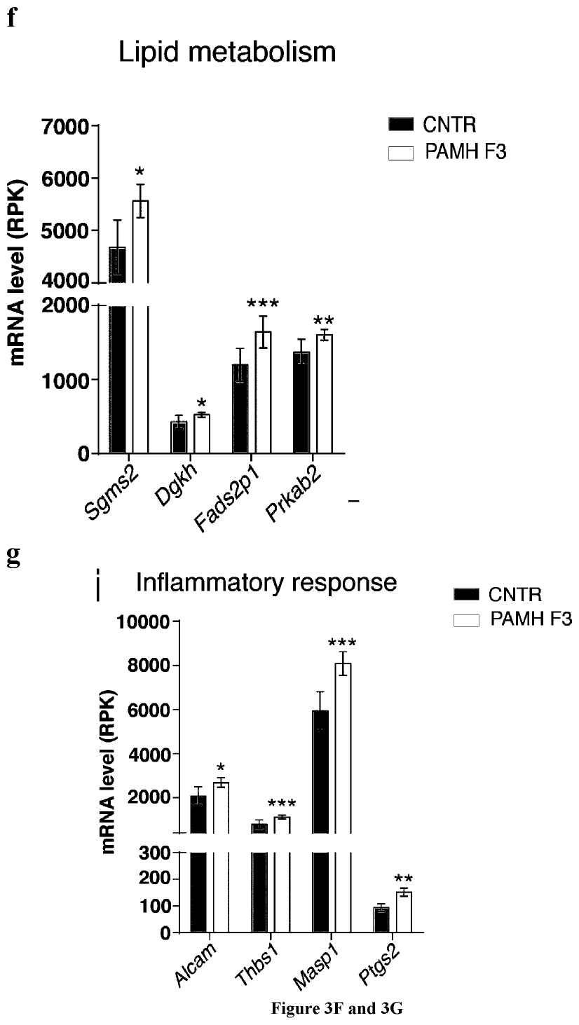

To dissect the molecular mechanisms and the affected gene pathways underlying ‘fetal reprogramming’ of PCOS, we performed RNAseq analysis in ovaries dissected from control dioestrous offspring (CNTR) and from PAMH F3 dioestrous and performed differential gene expression analysis (FIG. 3a, Data not shown). We identified 102 differentially expressed genes (DEGs; 54 downregulated and 48 upregulated; adjusted p value ≤0.05) in PAMH F3 ovaries compared to control ovaries, respectively (Data not shown). Next, we generated heat maps showing the expression patterns of the 102 DEGs in control and PAMH F3 offspring (Data not shown). Several differentially down-regulated genes are involved in regulating Insulin-like Growth Factor (IGF) transport and uptake by Insulin-like Growth Factor Binding Proteins (IGFBPs) as shown in the STRING protein interaction network (Data not shown).

To further get insights into gene function, we performed a gene enrichment analysis with the DEGs using the Database for Annotation, Visualization and Integrated Discovery (DAVID) functional annotation tool (p value ≤0.05; Data not shown). The downregulated genes-associated biological processes in PAMH F3 offspring are involved in DNA repair, cell cycle arrest, negative regulation of phosphorylation and negative regulation of cell proliferation (Data not shown). Pathway analyses were used to identify the significant pathways associated with the differentially expressed genes according to Kyoto Encyclopedia of Genes and Genomes (KEGG) (FIG. 3b, 3c). Our analysis revealed that among the downregulated genes, the most affected pathway is FoxO signaling pathway (FIG. 3b), which is related to the regulation of cell cycle and control of quiescence of primordial follicles, steroidogenesis in ovarian granulosa cells, apoptosis and insulin signaling (Dupont and Scaramuzzi, 2016; Richards and Pangas, 2010). The upregulated genes in the PAMH lineage are involved in axon guidance, fatty acid biosynthetic process, transforming growth factor beta (TGF-β) production and metabolic processes (Data not shown). KEGG pathway analysis showed that the most affected pathway among the upregulated genes is the TGF-β signaling pathway, which is involved in folliculogenesis, ovarian function, inflammation, glucose and energy homeostasis (Dupont and Scaramuzzi, 2016; Richards and Pangas, 2010) (FIG. 3c).

Interestingly, among the upregulated genes, we found some significant enrichment in PAMH F3 offspring of genes involved in the negative regulation of insulin secretion and in the control of folliculogenesis and ovarian steroidogenesis (Findlay, 1993; Poulsen et al., 2020), such as Inhibin b (Inhbb), Insulin Degrading Enzyme (Ide) (FIG. 3d) and Follistatin (Fst; FIG. 3e). Moreover, we identified a significant enrichment in the ovaries of PAMH F3 mice of genes involved in lipid metabolism (FIG. 3f) and inflammatory response (FIG. 3g).

The top 20 significant upregulated and downregulated genes by fold change in PAMH F3 ovaries versus control ovaries are presented in FIG. 4. The top upregulated genes in third-generation PCOS-like ovaries are mainly related to ovarian function, insulin metabolic process, inflammation, angiogenesis, cell cycle progression and axon guidance (FIG. 4a). The top 20 downregulated genes are mainly linked to epigenetic modifications, such as histone acetylation or methylation, apoptotic process, cell proliferation and regulation of cell cycle progression (FIG. 4b). Interestingly, the expression of 7 genes among the top 20 upregulated ones (FIG. 4a, asterisks) and 1 gene, among the top 20 downregulated ones (FIG. 4b), were previously reported to be altered in women with PCOS (Data not shown), strengthening the validity of our animal model.

To confirm our RNA-seq results, the expression of 6 upregulated genes and 6 downregulated genes related to ovarian function, metabolism, inflammation, axon guidance and cell migration was confirmed by RT-qPCR (Data not shown). The results of qPCR showed that the expression of related genes is in accordance with the RNA-seq analysis results (Data not shown).

These findings show that ancestral exposure to high intrauterine AMH drives ovarian transcriptomic changes into the third generation of the PAMH lineage that are associated to PCOS phenotypic reproductive and metabolic dysfunctions.

Alterations of DNA Methylation Patterns in Ovaries of PAMH Mice.

Since ancestral prenatal AMH exposure leads to alterations in ovarian gene expression in the third generation, we next investigated whether it could modulate the epigenome in PAMH F3 offspring. We used methylated DNA immunoprecipitation (anti-5′methyl-cytosine, 5mC) combined with deep sequencing (MeDIP-seq) to profile the methylomic landscape in control dioestrous ovaries (CNTR, prenatally PBS-treated) versus PAMH F3 dioestrous ovaries (Data not shown).

MeDIP efficiency was assessed using spike-in controls for unmethylated and methylated DNA regions from Arabidopsis thaliana (Data not shown). Principal component analysis, particularly the PC2, indicates an evident separation of CNTR and PAMH F3 groups (Data not shown).

We then calculated the differentially methylated windows between the two groups. Applying an adjusted p value ≤0.05 returned 185 significant hypermethylated regions and 887 hypomethylated regions in the ovaries of PAMH F3 offspring as compared to control offspring (Data not shown), corresponding to 173 exclusively hypermethylated genes and 858 hypomethylated genes.

We defined feature sets spanning sub-typed by location (exon, intergenic, intron, promoter-Transcription Start Site [TSS], Transcription Termination site [TTs]) of the hypomethylated and hypermethylated regions (Data not shown). We observed that the hypermethylated regions were mostly localized in intronic and intergenic regions, whereas hypomethylated regions were mostly found into upstream-promoters and TSS, thereby most likely affecting gene expression.

To determine whether DNA methylation changes might associate with gene expression changes, we looked for overlap between differentially methylated genes with DEGs of PAMH F3 ovaries (Data not shown). Four common genes between MeDIPseq and RNAseq were found: Roundabout homolog 1 (Robo-1), Sorbin and SH3 Domain-Containing Protein 2 (Sorbs2), Cyclin Dependent Kinase Inhibitor 1A (Cdkn1a), and Histidine Decarboxylase (Hdc) (Data not shown). They are respectively implicated in Slit/Robo pathway, Notch signaling, inhibition of cell proliferation and inflammation (Data not shown).

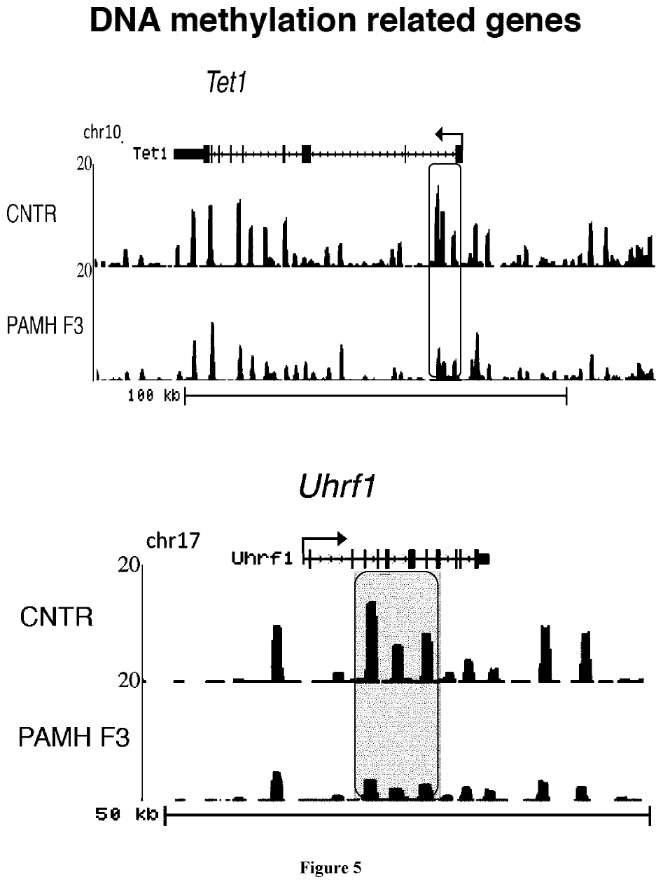

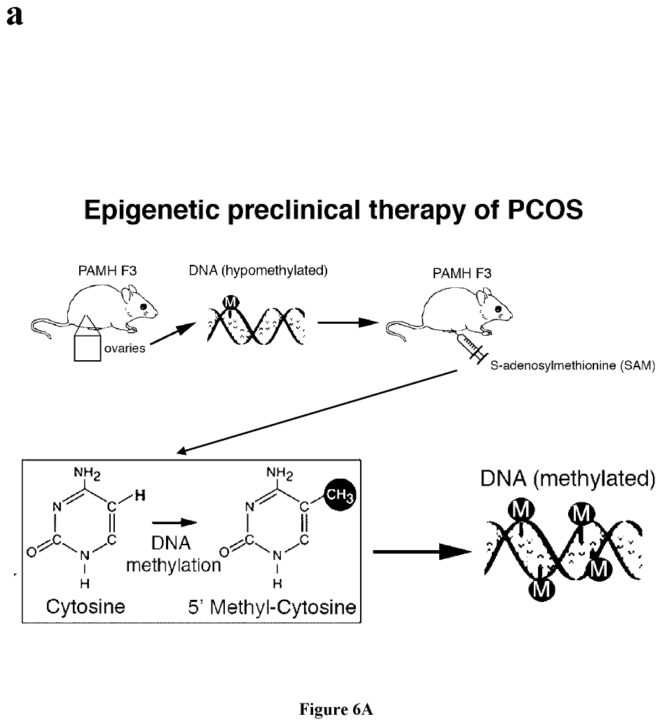

To begin defining the functional significance of the extensive changes in DNA methylation of third generation PCOS-like mice, GO-term enrichment analysis was performed and revealed distinct functional categories for the PAMH-associated gene lists (p value ≤0.05; FIG. 5). Within the category of biological processes (top 20 most significant processes) for the hypomethylated genes, chromatin remodeling and chromatin modification, cell cycle, cell differentiation, lipid metabolism, insulin response were listed in the top related functions (Data not shown). Regarding the KEGG pathways, the hypomethylated genes were enriched with metabolic pathways and type 2 diabetes (Data not shown). Genes associated with insulin regulation (glycolysis/gluconeogenesis and type 2 diabetes) are represented in a STRING protein network predicting interactions of those proteins associated with glucose metabolism, insulin signaling, insulin response and insulin receptor binding (Data not shown).

Within the category of GO biological processes for the hypermethylated genes, nervous system development, axons guidance, heart development, transcription and methylation pathways were listed in the top related functions (Data not shown). KEGG analysis revealed that GABAergic synapse pathway was significantly enriched in the hypermethylated genes (Data not shown). In accordance with these changes, preclinical investigations in PCOS animal models have reported an ovarian hyperinnervation and a potential contribution of the peripheral sympathetic system in the initiation and/or perpetuation of PCOS has been thus proposed (Stener-Victorin et al., 2005).

Depicting the differentially methylated genes along the chromosomes in a Manhattan plot confirmed a preponderance of hypomethylation in the PAMH F3 samples and indicated that epigenetic changes occur quite homogenously across all chromosomes (Data not shown).

Lastly, we identified significant changes in DNA methylation in the locuses of key genes involved in demethylase activities such as Ten-eleven translocation methylcytosine dioxygenase 1 (Tet1), and factors responsible for DNA methylation maintenance such as and Ubiquitin-like, containing PHD and RING finger domains, 1 (Uhrf1) in PAMH F3 ovaries compared to control ovaries (FIG. 5). Both Tet1 and Uhrf1 locuses were significantly hypomethylated in the third generation PCOS-like ovaries compared to controls (FIG. 5).

Consistently, Tet1 gene expression was found to be upregulated in our RNA-seq analysis of PAMH F3 ovaries vs control ovaries (p=0.009; Data not shown), even though the adjusted P value did not reach statistical significance (p=0.36; Data not shown).

Overall these experiments identified many genes and pathways associated to the PCOS phenotype, with altered DNA methylation profile in ovaries of the third generation of PAMH offspring. They highlight alteration in the insulin stimulus, glycolysis/gluconeogenesis and type 2 diabetes pathways, with a preponderance of hypomethylation in ovarian tissues of PCOS-like animals. These data also show that DNA methylation related genes are hypomethylated and they could thus be responsible for the global loss of methylation detected in PCOS mice.

Methyl Donor S-Adenosylmethionine (SAM) Treatment of PAMH F3 Mice Normalizes their Neuroendocrine Reproductive and Metabolic Phenotype.

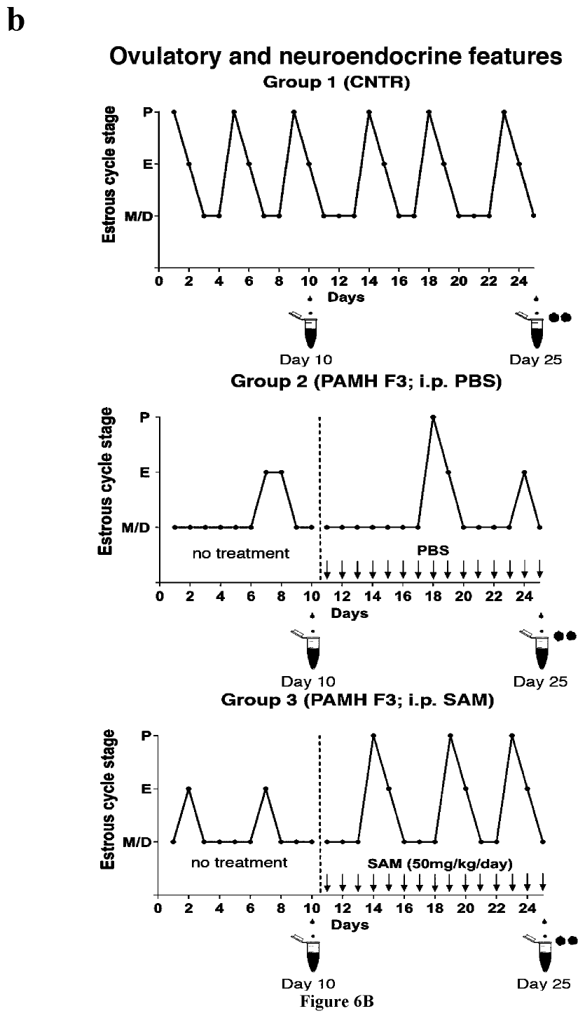

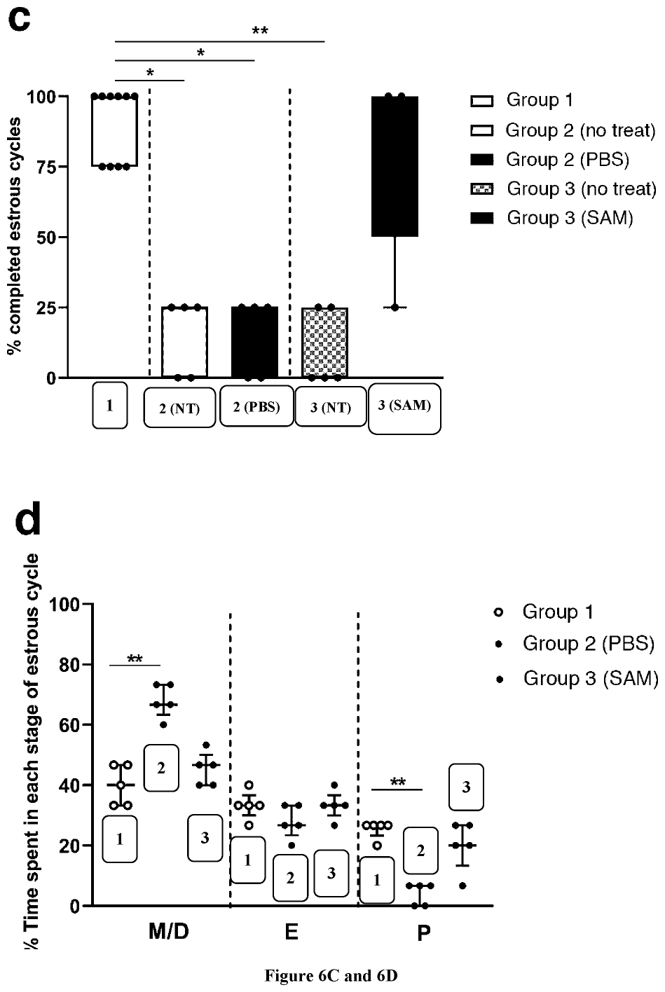

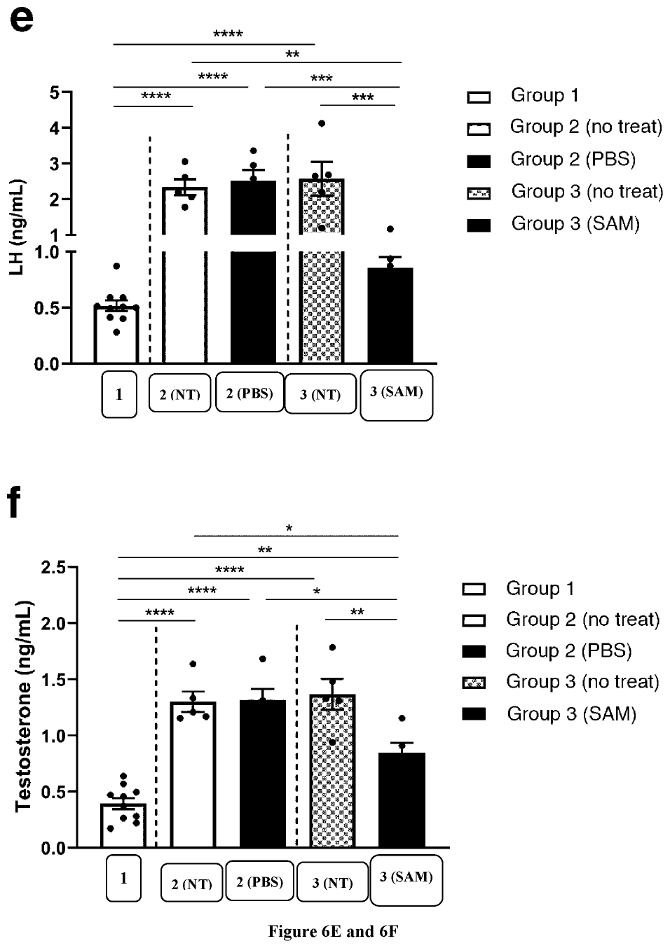

Since our MeDIP-seq analyses pointed to a preponderance of hypomethylation in ovarian tissues of PCOS-like animals compared to control animals, we then examined the therapeutic potential of using the universal methyl group donor S-adenosylmethionine (SAM) in an epigenetic preclinical investigation (FIG. 6a).

SAM is an important and naturally occurring biomolecule found ubiquitously in all living cells and functions as the primary methyl donor for all transmethylation reactions (Bottiglieri, 2002) and can thereby be used to promote methylation of otherwise hypomethylated tissues (FIG. 6a).

In the present study, we first analyzed estrous cyclicity of adult control (CNTR; 6 months-old Group 1) and PAMH F3 offspring (6 months-old) for 25 and 10 days, respectively, to confirm the oligo-anovulatory phenotype of PCOS-like animals (FIG. 6b). Thereafter, we monitored for additional 15 days vaginal cytology of PAMH F3 animals treated either with i.p. injections of PBS (Group 2) or with 50 mg/kg daily injections of SAM (Group 3). Tail-blood samples were collected for LH and T measurements at dioestrus (day 10), before the beginning of the treatment, and trunk-blood and ovaries were collected at day 25 (dioestrus) at the moment of the sacrifice, corresponding to the end of the treatment period (FIG. 6b). As expected, PAMH F3 mice of the group 2 displayed between 10 and 25% of completed estrous cycles during the monitoring time, either before or during the treatment (PBS), as compared to control mice (FIG. 6c). PAMH F3 animals injected with SAM displayed a significant increase in the percentage of completed estrous cycles, reaching 75% of completed estrous cycles (Group 3; FIG. 6c). We then quantified the percentage of time that animals spent in each cycle stage and showed that while PAMH F3 animals of the Group 2 displayed prolonged time in metestrus and dioestrus as compared to control offspring, SAM treatment restored normal ovulation of PCOS animals of Group 3 (FIG. 6d). We also assessed whether SAM treatment could ameliorate the PCOS-like neuroendocrine phenotype of these animals. The aberrant LH and T concentrations, typical of PAMH mice, were also normalized by this treatment (FIG. 6e, f). Lastly, the epigenetic pharmacological treatment also normalized the body mass composition (% of fat mass and % of lean mass) of PAMH F3 offspring to control conditions (FIG. 6g).

These data show that postnatal SAM treatment can rescue the major PCOS-like neuroendocrine, reproductive and metabolic traits of PAMH F3 mice.

Islet cell hyperplasia has been linked with type 2 diabetes in the leptin deficient ob/ob mouse, which has been extensively studied as a model for this disease for decades and which couples insulin resistance and obesity with a marked expansion of p-cell mass to compensate for increased insulin demand (Bock et al., 2003). Interestingly, we detected a marked increase in the volume of the islets of Langerhans in 6 months-old PAMH F1 female mice (Data not shown), consistent with a diabetic status of our animal model. Moreover, we observed that the pancreatic islet hyperplasia detected in PAMH F1 animals was transgenerationally passed to the third generation of PAMH animals and that the SAM treatment normalized the size of the islet of Langerhans in these mice (FIG. 6h).

To further explore the effect of the SAM treatment on DNA methylation and gene expression levels, we harvested the ovaries from CNTR, PAMH F3 and PAMH F3-SAM animals at the end of the treatment period and performed qRT-PCR experiments (Data not shown).

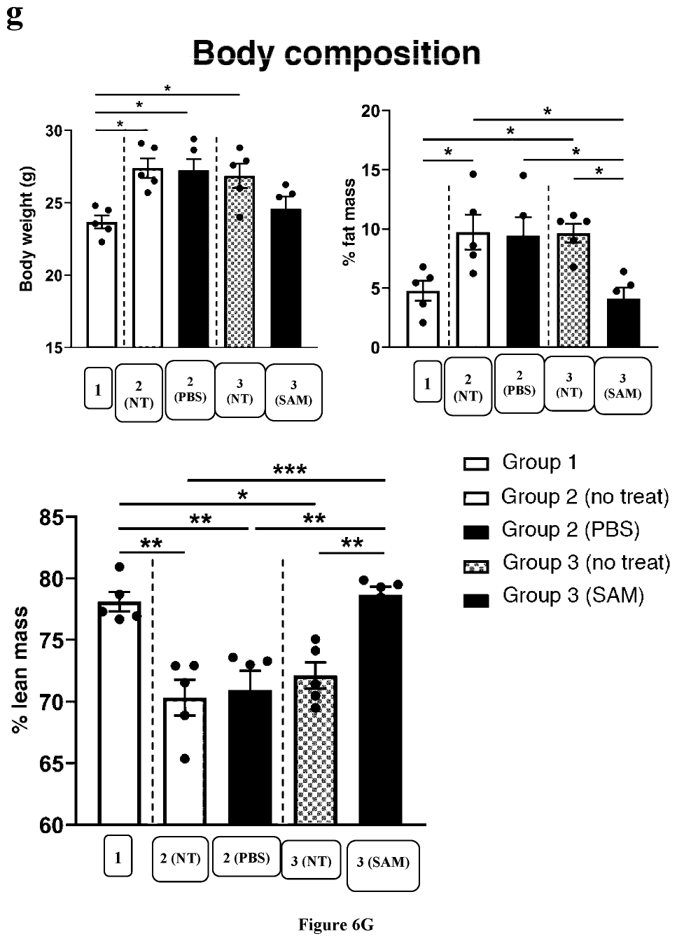

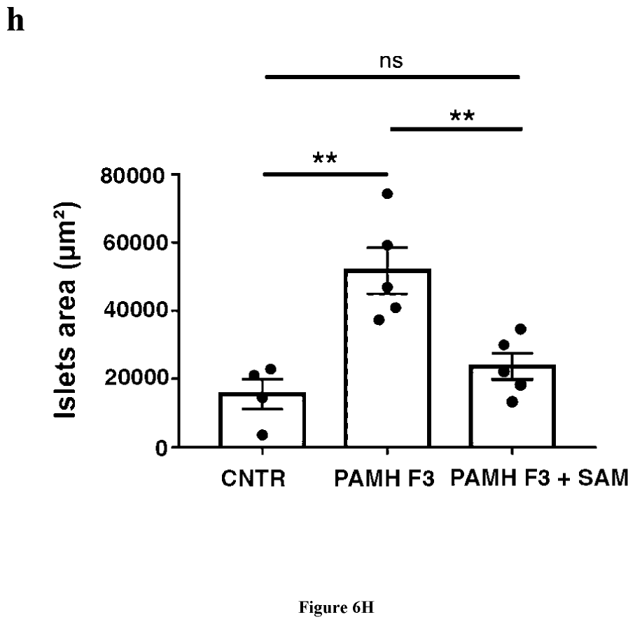

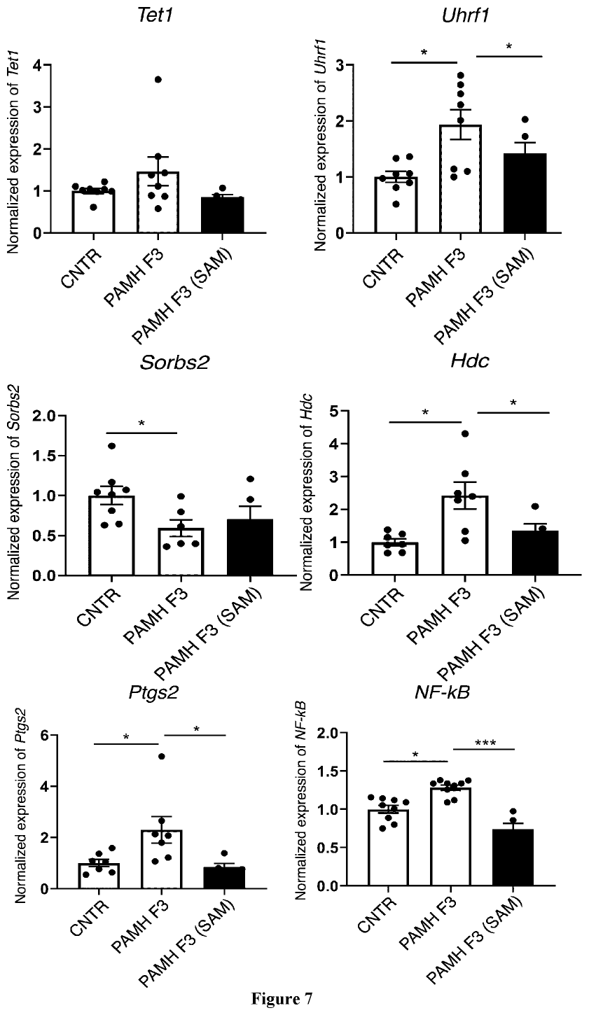

We first analyzed by RT-qPCR the transcript expression levels of the DNA methylation related genes, Tet1 and Uhrf1, which were significantly hypomethylated in PAMH F3 ovaries (FIG. 7). While Tet1 transcript levels were unaltered in ovarian tissues of PAMH F3 animals, either treated or not with the epigenetic drug, as compared with controls, Uhrf1 was significantly upregulated in PAMH F3 ovaries (FIG. 7). Notably, SAM treatment restored Uhrf1 expression in PAMH F3 mice to normal conditions (FIG. 7). We also selected two ovarian genes that we found both differentially expressed and methylated in PAMH F3 offspring versus controls, namely Sorbs2 and Hdc, related respectively to Notch signaling and inflammatory response. From our MeDIP-seq and RNAseq analyses, Sorbs2 resulted to be hypermethylated while its transcript levels were down-regulated in PAMH F3 ovaries versus CNTR (Data not shown). Our RT-qPCR experiments confirmed a significant down-regulation of Sorbs2 in the ovaries of PCOS animals, while its expression remained unaltered after the SAM treatment (FIG. 7). These results indicate that, in principle, the primary methyl donor SAM does not impact the transcript expression of hypermethylated genes.

Consistently with our RNAseq analysis, we identified a two-fold increase in Hdc transcript levels in PAMH F3 ovaries as compared with control animals (FIG. 7). Interestingly, the epigenetic treatment was able to rescue the altered ovarian gene expression of Hdc in PCOS-like animals (FIG. 7).

We lastly investigated the expression changes of two additional genes involved in ovarian inflammation, Ptgs2, which was hypomethylated in PAMH F3 ovaries (Data not shown), and Nuclear factor kappab (NF-κB), a known mediator of inflammation. RT-qPCR experiments showed that the mRNAs of both genes were upregulated in PAMH F3 ovaries and normalized in these tissues upon SAM treatment (FIG. 7).

Our data indicate that the mechanisms underlying SAM-regulated improvements in ovarian and metabolic dysfunction associated with PCOS likely involve restoration of gene expression of Uhrf1, which is required for DNA methylation maintenance. This is turn may be responsible for the normalization of aberrant expression of inflammatory genes, thus restoring metabolic and ovarian functions in PCOS animals (Data not shown).

Common Epigenetic Signatures in Ovarian Tissue of PAMH Lineage and Blood of Women with PCOS.

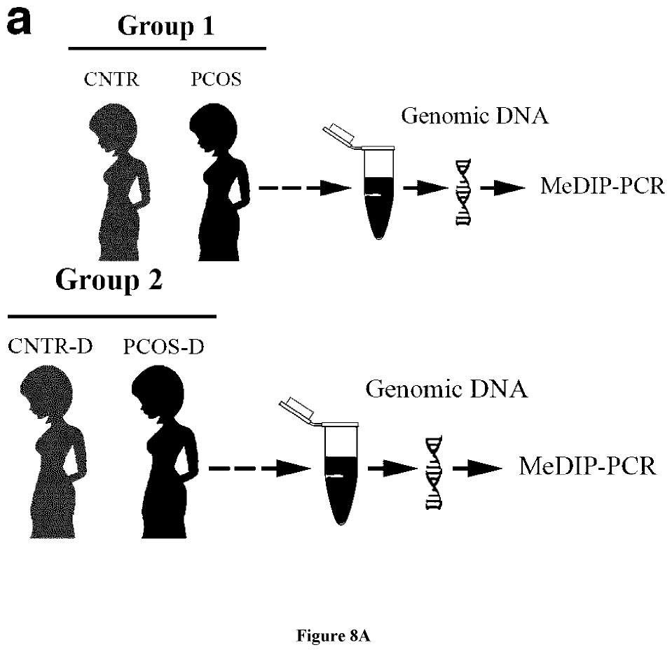

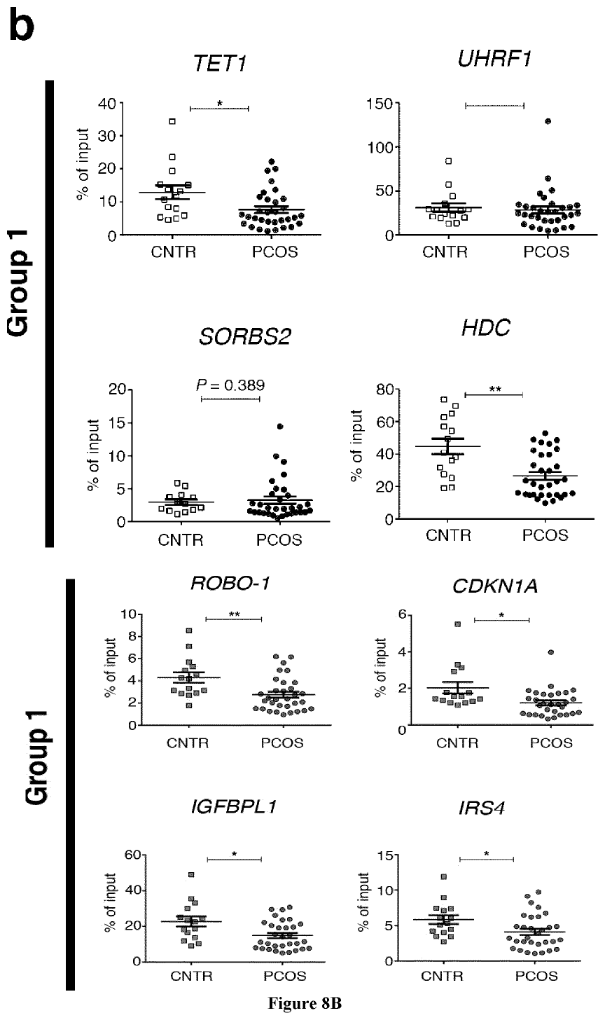

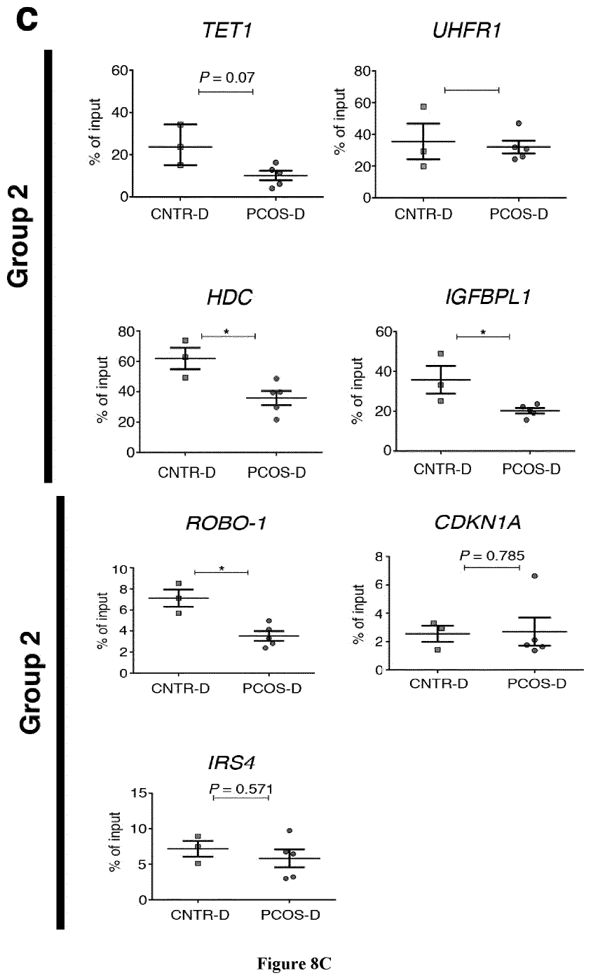

To investigate how our findings in mice might relate to the human PCOS condition, we searched by MeDIP-PCR for common epigenetic signatures in blood samples of PCOS women and control women (CNTR) as well as in post-pubertal daughters born to mothers with (PCOS-D) or without PCOS (CNTR-D; FIG. 8a, Data not shown). MeDIP efficiency was assessed using primers directed against Glyceraldehyde 3-phosphate dehydrogenase (GAPDH), as negative control, and the testicular gene Testis-Specific Histone H2B (TSH2B), as a positive control (Data not shown). Our experiments showed a strong hypomethylation of GAPDH and hypermethylation of TSH2B confirming the efficiency of the immunoprecipitation (Data not shown).

Taking into account the hypomethylation of two key DNA methylation related genes in PAMH F3 ovaries, Tet1 and Uhrf1, that could account for the preponderance of global DNA hypomethylation that we identified in PCOS-like mice, we first assessed the methylation levels of TET1 and UHRF1 in blood samples of women with PCOS and in control women. Interestingly, TET1 was significantly hypomethylated in PCOS women as compared with controls, whereas methylation levels of UHRF1 were comparable in the two groups (FIG. 8b).

We next selected the four ovarian genes that were differentially expressed and methylated in the PAMH F3 offspring versus controls, namely Robo-1, Sorbs2, Cdkn1a and Hdc and 2 additional hypomethylated genes, which may be related to defects in insulin signaling in PCOS: Insulin Like Growth Factor Binding Protein Like 1 (IGFBPL1) and Insulin Receptor Substrate 4 (IRS4). Five (ROBO-1, CDKN1A, HDC, IGFBPL1, IRS4) out of the 6 genes selected from the genome-wide methylation profile of the PAMH lineage were identified as being also differentially methylated in the blood samples of women with PCOS as compared with healthy women (FIG. 8b). Notably, all changes were identified in the promoter regions of these genes.

ROBO-1, HDC and IGFBPL1 were hypomethylated also in blood samples of post-pubertal daughters, diagnosed with PCOS, and born to mothers with PCOS as compared to control daughters (FIG. 8c). PCOS daughters also showed a tendency to lower methylation levels of TET1 as compared with controls, even though it did not reach statistical significance, due to the low number of subjects (FIG. 8c).

These findings highlighted the existence of common epigenetic signatures in our PCOS preclinical model and in women with PCOS and identify methylome markers, which are common in women with PCOS and in daughters from mothers with PCOS.

Discussion

Familial clustering and twin studies have shown that PCOS has a strong heritable component (McAllister et al., 2015). However, the PCOS loci identified by genome-wide association studies account for less than 10% of heritability (Azziz, 2016), suggesting that environmental and epigenetic mechanisms may play an important role in the etiology of this disease. Preclinical and clinical investigations have pointed to altered levels of androgens or AMH during pregnancy as the culprit for the fetal programming of PCOS (Stener-Victorin et al., 2020; Walters et al., 2018a; Walters et al., 2018b). Hence, prenatally androgen treated (PNA) and AMH treated (PAMH) animals are excellent preclinical models to mimic a key maternal PCOS condition in which to investigate whether exposed lineages have increased susceptibility to a PCOS-like reproductive and metabolic phenotype in F1 to F3 offspring (Stener-Victorin et al., 2020). Consistently, a recent study assessed transgenerational transmission of PCOS-like phenotypes in prenatally andogenized (PNA) mice (Risal et al., 2019). However, the mechanisms underlying the inheritance and transmission of PCOS-traits to subsequent generations have not been elucidated. To further explore the field of transgenerational transmission of PCOS and dissect the cascade of molecular events leading to increased disease susceptibility, we used the PAMH mouse model, which recapitulates the major neuroendocrine reproductive traits of PCOS (Tata et al., 2018). Moreover, we showed that as PAMH mice age they acquire a metabolic phenotype which mimics the human pathological condition, including increased fasting glucose level, impaired glucose tolerance and impaired insulin sensitivity. These data are consistent with the observation that as women with PCOS age, an increased incidence of type 2 diabetes mellitus emerges (Wild et al., 2010).

Here we showed that PAMH animals pass to subsequent generations all the major diagnostic features of PCOS in women: hyperandrogenism, ovulatory dysfunctions and altered fertility, together with metabolic dysfunctions, which are also a common feature in many women with PCOS (Stener-Victorin et al., 2020). Importantly, all these defects are maintained for at least three generations, making the PAMH mouse an amenable preclinical model to study mechanistic aspects underlying the transmission of reproductive and metabolic traits of PCOS.

Genetic and epigenetic modifications have been demonstrated to be implicated in the transgenerational inheritance of prenatally programmed diseases (Cavalli and Heard, 2019; Gapp et al., 2014). Our findings suggest that prenatal aberrant AMH exposure has an adverse and long-term effect on ovarian gene expression, associated with ovarian dysfunctions, in PAMH F3 offspring. This is particularly relevant, since we and others have recently shown that women with PCOS have higher levels of circulating AMH during pregnancy compared to non-PCOS pregnant women (Piltonen et al., 2019; Tata et al., 2018).

Despite differences in ovarian morphology and physiology between mice and humans, our data show that the ovarian gene expression is globally conserved between these species. Among the upregulated genes, we found some significant enrichment in PCOS mice of genes involved in the negative regulation of insulin secretion, including Inhbb, Ide, Fst and TGF-β signaling pathway, which also regulate folliculogenesis and ovarian steroidogenesis (Findlay, 1993; Liu et al., 2016). Accordingly, increase in FST may arrest follicular development and drive ovarian androgen production, both of which are typical traits of PCOS. Activin A and FST are also directly involved in promotion and regulation of inflammation, which has been implicated in the onset of insulin-resistance and diabetes (Sjoholm and Nystrom, 2006).

Consistent with those studies, we identified in the ovaries of PAMH F3 mice (at P60) a significant enrichment of genes involved in both inflammatory response and insulin-resistance/diabetes (Data not shown), even before the appearance of phenotypic manifestation of diabetes in these animals, occurring few months later. These pathways are known to be commonly affected in PCOS ovarian tissue dysfunction (Liu et al., 2016; Pan et al., 2018).

Finally, the expression of 8 genes among the top DEGs (Grem1, Ide, Ptgs2, Thbs1, Aqp8, Fst, Inhbb, Cdkn1a), and/or of their products, were previously reported to be altered in women with PCOS (Chen et al., 2010; Jiang et al., 2015; Liu et al., 2015; Liu et al., 2016; Wachs et al., 2006; Wang et al., 2008; Xiong et al., 2019), further supporting the validity of our animal model.

Epigenetic modifications work in concert with genetic machinery to regulate transcriptional activity in normal tissues and are often dysregulated in disease (Kelly et al., 2010). In recent years, epigenetic factors have gained considerable attention in the study of the PCOS pathogenesis (Escobar-Morreale, 2018; Makrinou et al., 2020; Patel, 2018; Tata et al., 2018; Vazquez-Martinez et al., 2019). Here, we identified many differentially methylated genes in PAMH F3 ovaries associated with the PCOS phenotype. Interestingly, we observed a preponderance of hypomethylation in ovarian tissues of these animals. A global loss of DNA methylation, particularly in promoter-TSS and upstream-promoters, as the ones detected in this study, could be responsible for genomic instability in the disease condition. In agreement with our findings, a genome-wide DNA methylation study on umbilical cord blood, reported a prevalence of hypomethylation in women with PCOS compared with unaffected women (Lambertini et al., 2017).

Notably, our MeDIP-seq experiments showed that the most affected genes in the ovarian tissue of PCOS animals were correlated with metabolic pathways and type 2 diabetes, which are known to be affected in PCOS women (Boyle and Teede, 2016; Dumesic et al., 2015; Vazquez-Martinez et al., 2019). These alterations are in accordance with the hyperandrogenism and ovulatory dysfunctions as well as metabolic alterations of PCOS women and PAMH animals. Indeed, both LH hypersecretion and hyperinsulinemia are known to exacerbate ovarian theca cell androgen production (Franks, 2008). Consistent with these findings, genome-wide DNA methylation studies indicate that differentially methylated genes in various tissues of women with PCOS are related to inflammation, hormone-related processes, glucose and lipid metabolism (Makrinou et al., 2020; Shen et al., 2013; Vazquez-Martinez et al., 2019).

Interestingly, the main biological processes associated with hypomethylated genes were related to chromatin remodeling and covalent chromatin modification, suggesting that transgenerationally transmitted alterations in PCOS animals may widely impact chromatin organization. Mechanistically, we identified a significant hypomethylation in the locus of two key genes involved in DNA demethylation (Tet1) and DNA methylation maintenance (Uhrf) in PAMH F3 ovaries compared to control ovaries of which our RNA-seq data showed increased Tet1 expression with a significant p value, which could account for the DNA methylation loss in PAMH F3 ovaries.

At the functional level, we report four genes that exhibited alterations both in DNA methylation and mRNA expression. Three of those genes, Robo-1, Sorbs2 and Cdkn1a, are regulators of ovarian function. The fourth common gene, Hdc, is implicated in inflammatory response. Based on the canonical view of 5mC being a repressor of transcription (Deaton and Bird, 2011), our results indicate a mismatch between the methylation state and the level of gene expression for Robo-1 and Cdkn1a. However, this is not a general rule, as the mechanism by which DNA methylation regulates transcription can be specific to different contexts such as gene content, locus, and developmental timing (Tremblay and Jiang, 2019). Since DNA methylation is actually more abundant in gene bodies, a primary role for DNA methylation could be to fine tune levels of expression and splicing, rather than acting as an on-off switch at gene promoters (Tremblay and Jiang, 2019). Considering that the most hypomethylated genes emerging from our analysis were related to chromatin remodeling and chromatin modification, it is possible that, besides DNA methylation, other epigenetic events, such as histone acetylation/methylation are modulated altering gene expression, which could in part explain the weak correlation that we observed between MeDIP-seq and RNA-seq. In line with these findings, histone acetylation alteration has been reported in various tissues of women with the disease (Qu et al., 2012; Vazquez-Martinez et al., 2019).

Remarkably, we report that several of the differentially methylated genes identified in ovarian tissues of PCOS mice of the third generation were also altered in blood samples from women with PCOS and from daughters of women with PCOS compared with healthy women. In particular, TET1 was significantly hypomethylated in women with PCOS as compared with control women and a tendency to a hypomethylation of this gene was also observed in PCOS-daughters. Since TET1 is one of the family members of 5mC dioxygenases, which oxidize 5mC and initiate demethylation, it is likely that the decreased levels of TET1 methylation observed in PCOS women could be at the origin of the preponderance of global DNA hypomethylation characterizing the disease and of the molecular and phenotypic alterations associated with PCOS.

Five genes out of the 6 selected from the genome-wide methylation and RNA sequencing, ROBO-1, CDKN1A, HDC, IGFBPL1 and IRS4, respectively associated with axon guidance, inflammation and insulin signaling, were found to be hypomethylated in women with PCOS as compared with controls and three genes (ROBO-1, HDC, IGFBPL1) were also found hypomethylated in daughters diagnosed with PCOS. Since the BMI of PCOS women was found not significantly different compared to controls both in unrelated women and in CNTR-D and PCOS-D (Data not shown) and because several metabolic parameters (fasting insulin, fasting glycemia and triglycerides) were within the normal range in the PCOS groups, we can exclude a priori the contribution of metabolic alterations on the differential methylation landscape of women with PCOS versus control women.

Because DNA methylation epigenetic changes can precede phenotypic manifestation and display more stability than gene expression alterations (Kelly et al., 2010), the differentially methylated genes offer the opportunity to develop valuable diagnostic indicators for PCOS risk or prognostic indicators for the disease progression.

Even more importantly, the reversible nature of epigenetic modifications makes them more ‘druggable’ than attempts to target or correct defects in gene expression itself. Both hypermethylation and hypomethylation are involved in several disease conditions (Kelly et al., 2010). Nevertheless, most of the scientific interest in epigenetic studies for the past two decades focused primarily on hypermethylation. As a consequence, several inhibitors of DNA methylation are currently approved for many pathologies by the Food and Drug Administration (FDA) and have been in clinical use for several years (Kelly et al., 2010). However, at present, there are no FDA-approved therapeutic modalities that target hypomethylation. Importantly, we showed a preponderance of hypomethylation in ovarian tissues of PCOS-like animals. Moreover, we and others (Lambertini et al., 2017) found hypomethylation signatures in the peripheral blood of women with PCOS, which led us to examine the therapeutic potential of SAM, a known agent causing methylation of several genes (Chik et al., 2014). Our preclinical investigation showed that SAM treatment can rescue the major PCOS reproductive neuroendocrine and metabolic alterations of PAMH F3 mice thus highlighting the therapeutic potential of methylating agents as promising epigenetic therapies aimed at treating women with PCOS.

From a mechanistic point of view, the treatment with the methyl donor agent was able to rescue the expression of Uhrf1, which plays an important role in maintaining DNA methylation. This mechanism may in turn be responsible for the normalization of gene expression of several molecules involved in inflammation, which are overexpressed in PAMH F3 ovaries. Among these genes, we identified Ptgs2 and NF-κB, whose gene expressions are upregulated in PAMH F3 ovaries and normalized by the epigenetic treatment. Consistently, these genes were previously reported to be significantly higher in PCOS than in control women (Gao et al., 2016; Liu et al., 2016).

Evidence is accumulating, indicating an association of PCOS with chronic inflammation. However, the mechanisms underlying the increased levels of inflammatory markers in women with PCOS are still uncertain (Gao et al., 2016; Gonzalez et al., 2006; Stener-Victorin et al., 2020; Zhao et al., 2015). Based on our findings, we put forward the hypothesis that the PCOS proinflammatory state could result from an altered epigenetic landscape, which leads to overexpression of several genes involved in ovarian inflammation (data not shown). Chronic low-grade inflammation is known to promote insulin resistance and hyperandrogenism related to PCOS (Gonzalez et al., 2006; Zhao et al., 2015). Therefore, inflammation per se may be the trigger of both the metabolic and ovarian phenotype of the disease. Consistent with this hypothesis, anti-inflammatory therapy of peripubertal letrozole-induced PCOS-like animal models largely reversed hyperandrogenemia as well as reproductive and metabolic PCOS-like traits (Lang et al., 2019).

Taken together, this study points to AMH excess during gestation as a detrimental factor leading to the transgenerational transmission of PCOS cardinal neuroendocrine, reproductive and metabolic alterations and shed lights into the epigenetic modifications underlying the susceptibility of the disease while pointing to novel epigenetic-based therapeutic avenues to treat the disease.

TABLE 1
Useful nucleotide and amino acid sequences for practicing the invention

| SEQ ID | |
|---|---|
| NO | Nucleotide or amino acid sequence |
| 1 (TET1 | MSRSRHARPSRLVRKEDVNKKKKNSQLRKTTKGANKNVASVKTLSPG |
| AA | KLKQLIQERDVKKKTEPKPPVPVRSLLTRAGAARMNLDRTEVLFQNPES |
| sequence | LTCNGFTMALRSTSLSRRLSQPPLVVAKSKKVPLSKGLEKQHDCDYKIL |
| human) | PALGVKHSENDSVPMQDTQVLPDIETLIGVQNPSLLKGKSQETTQFWSQ |
| | RVEDSKINIPTHSGPAAEILPGPLEGTRCGEGLFSEETLNDTSGSPKMFAQ |
| | DTVCAPFPQRATPKVTSQGNPSIQLEELGSRVESLKLSDSYLDPIKSEHD |
| | CYPTSSLNKVIPDLNLRNCLALGGSTSPTSVIKFLLAGSKQATLGAKPDH |
| | QEAFEATANQQEVSDTTSFLGQAFGAIPHQWELPGADPVHGEALGETP |
| | DLPEIPGAIPVQGEVFGTILDQQETLGMSGSVVPDLPVFLPVPPNPIATFN |
| | APSKWPEPQSTVSYGLAVQGAIQILPLGSGHTPQSSSNSEKNSLPPVMAI |
| | SNVENEKQVHISFLPANTQGFPLAPERGLFHASLGIAQLSQAGPSKSDRG |
| | SSQVSVTSTVHVVNTTVVTMPVPMVSTSSSSYTTLLPTLEKKKRKRCGV |
| | CEPCQQKTNCGECTYCKNRKNSHQICKKRKCEELKKKPSVVVPLEVIKE |
| | NKRPQREKKPKVLKADFDNKPVNGPKSESMDYSRCGHGEEQKLELNP |
| | HTVENVTKNEDSMTGIEVEKWTQNKKSQLTDHVKGDFSANVPEAEKS |
| | KNSEVDKKRTKSPKLFVQTVRNGIKHVHCLPAETNVSFKKFNIEEFGKT |
| | LENNSYKFLKDTANHKNAMSSVATDMSCDHLKGRSNVLVFQQPGFNC |
| | SSIPHSSHSIINHHASIHNEGDQPKTPENIPSKEPKDGSPVQPSLLSLMKDR |
| | RLTLEQVVAIEALTQLSEAPSENSSPSKSEKDEESEQRTASLLNSCKAILY |
| | TVRKDLQDPNLQGEPPKLNHCPSLEKQSSCNTVVFNGQTTTLSNSHINS |
| | ATNQASTKSHEYSKVTNSLSLFIPKSNSSKIDTNKSIAQGIITLDNCSNDL |
| | HQLPPRNNEVEYCNQLLDSSKKLDSDDLSCQDATHTQIEEDVATQLTQL |
| | ASIIKINYIKPEDKKVESTPTSLVTCNVQQKYNQEKGTIQQKPPSSVHNN |
| | HGSSLTKQKNPTQKKTKSTPSRDRRKKKPTVVSYQENDRQKWEKLSY |
| | MYGTICDIWIASKFQNFGQFCPHDFPTVFGKISSSTKIWKPLAQTRSIMQ |
| | PKTVFPPLTQIKLQRYPESAEEKVKVEPLDSLSLFHLKTESNGKAFTDKA |
| | YNSQVQLTVNANQKAHPLTQPSSPPNQCANVMAGDDQIRFQQVVKEQ |
| | LMHQRLPTLPGISHETPLPESALTLRNVNVVCSGGITVVSTKSEEEVCSS |
| | SFGTSEFSTVDSAQKNFNDYAMNFFTNPTKNLVSITKDSELPTCSCLDRV |
| | IQKDKGPYYTHLGAGPSVAAVREIMENRYGQKGNAIRIEIVVYTGKEGK |
| | SSHGCPIAKWVLRRSSDEEKVLCLVRQRTGHHCPTAVMVVLIMVWDGI |
| | PLPMADRLYTELTENLKSYNGHPTDRRCTLNENRTCTCQGIDPETCGAS |
| | FSFGCSWSMYFNGCKFGRSPSPRRFRIDPSSPLHEKNLEDNLQSLATRLA |
| | PIYKQYAPVAYQNQVEYENVARECRLGSKEGRPFSGVTACLDFCAHPH |
| | RDIHNMNNGSTVVCTLTREDNRSLGVIPQDEQLHVLPLYKLSDTDEFGS |
| | KEGMEAKIKSGAIEVLAPRRKKRTCFTQPVPRSGKKRAAMMTEVLAHK |
| | IRAVEKKPIPRIKRKNNSTTTNNSKPSSLPTLGSNTETVQPEVKSETEPHFI |
| | LKSSDNTKTYSLMPSAPHPVKEASPGFSWSPKTASATPAPLKNDATASC |
| | GFSERSSTPHCTMPSGRLSGANAAAADGPGISQLGEVAPLPTLSAPVME |
| | PLINSEPSTGVTEPLTPHQPNHQPSFLTSPQDLASSPMEEDEQHSEADEPP |
| | SDEPLSDDPLSPAEEKLPHIDEYWSDSEHIFLDANIGGVAIAPAHGSVLIE |
| | CARRELHATTPVEHPNRNHPTRLSLVFYQHKNLNKPQHGFELNKIKFEA |
| | KEAKNKKMKASEQKDQAANEGPEQSSEVNELNQIPSHKALTLTHDNV |
| | VTVSPYALTHVAGPYNHWV |
| 2 (TET1 | actccctgaggtctgtcctggggagacactgctgctccggggggctgacctggggggagtggccgcgcagtc |
| nucleic | tgctccggcgccgctttgtgcgcgcagccgctggcccctctactcccgggtctgccccccgggacacccctctgc |
| acid | ctcgcccaagtcatgcagccctacctgcctctccactgtggacctttgggaaccgactcctcacctcgggggctcg |
| sequence | ggccttgactgtgctgggagccggtaggcgtcctccgcgaccegcccgcgcccctegcgcccgccggggccc |
| human: | cgggctccaaagttgtggggaccggcgcgagttggaaagtttgcccgagggctggtgcaggcttggagctggg |
| mRNA) | ggccgtgcgctgccctgggaatgtgacccggccagcgaccaaaaccttgtgtgactgagctgaagagcagtgc |
| | atccagattctcctcagaagtgagactttccaaaggaccaatgactctgtttcctgcgccctttcattttttcctactct |
| | gtagctatgtctcgatcccgccatgcaaggccttccagattagtcaggaaggaagatgtaaacaaaaaaaagaaaa |
| | acagccaactacgaaagacaaccaagggagccaacaaaaatgtggcatcagtcaagactttaagccctggaaa |
| | attaaagcaattaattcaagaaagagatgttaagaaaaaaacagaacctaaaccacccgtgccagtcagaagcctt |
| | ctgacaagagctggagcagcacgcatgaatttggataggactgaggttctttttcagaacccagagtccttaacctg |
| | caatgggtttacaatggcgctacgaagcacctctcttagcaggcgactctcccaacccccactggtogtagccaaa |
| | tccaaaaaggttccactttctaagggtttagaaaagcaacatgattgtgattataagatactccctgctttgggagtaa |
| | agcactcagaaaatgattcggttccaatgcaagacacccaagtccttcctgatatagagactctaattggtgtacaa |
| | aatccctctttacttaaaggtaagagccaagagacaactcagttttggtcccaaagagttgaggattccaagatcaat |
| | atccctacccacagtggccctgcagctgagatccttcctgggccactggaagggacacgctgtggtgaaggact |
| | attctctgaagagacattgaatgataccagtggttccccaaaaatgtttgctcaggacacagtgtgtgctccttttccc |
| | caaagagcaacccccaaagttacctctcaaggaaaccccagcattcagttagaagagttgggttcacgagtagaa |
| | tctcttaagttatctgattcttacctggatcccattaaaagtgaacatgattgctaccccacctccagtcttaataaggtt |
| | atacctgacttgaaccttagaaactgcttggctcttggtgggtctacgtctcctacctctgtaataaaattcctcttggc |
| | aggctcaaaacaagcgacccttggtgctaaaccagatcatcaagaggccttcgaagctactgcaaatcaacagg |
| | aagtttctgataccacctctttcctaggacaggcctttggtgctatcccacatcaatgggaacttcctggtgctgaccc |
| | agttcatggtgaggccctgggtgagaccccagatctaccagagattcctggtgctattccagtccaaggagaggt |
| | ctttggtactattttagaccaacaagaaactcttggtatgagtgggagtgttgtcccagacttgcctgtcttccttcctgt |
| | tcctccaaatccaattgctacctttaatgctccttccaaatggcctgagccccaaagcactgtctcatatggacttgca |
| | gtccagggtgctatacagattttgcctttgggctcaggacacactcctcaatcatcatcaaactcagagaaaaattca |
| | ttacctccagtaatggctataagcaatgtagaaaatgagaagcaggttcatataagcttcctgccagctaacactca |
| | ggggttcccattagcccctgagagaggactcttccatgcttcactgggtatagcccaactctctcaggctggtccta |
| | gcaaatcagacagagggagctcccaggtcagtgtaaccagcacagttcatgttgtcaacaccacagtggtgacta |
| | tgccagtgccaatggtcagtacctcctcttcttcctataccactttgctaccgactttggaaaagaagaaaagaaagc |
| | gatgtggggtctgtgaaccctgccagcagaagaccaactgtggtgaatgcacttactgcaagaacagaaagaac |
| | agccatcagatctgtaagaaaagaaaatgtgaggagctgaaaaagaaaccatctgttgttgtgcctctggaggttat |
| | aaaggaaaacaagaggccccagagggaaaagaagcccaaagttttaaaggcagattttgacaacaaaccagta |
| | aatggccccaagtcagaatccatggactacagtagatgtggtcatggggaagaacaaaaattggaattgaaccca |
| | catactgttgaaaatgtaactaaaaatgaagacagcatgacaggcatcgaggtggagaagtggacacaaaacaa |
| | gaaatcacagttaactgatcacgtgaaaggagattttagtgctaatgtcccagaagctgaaaaatcgaaaaactctg |
| | aagttgacaagaaacgaaccaaatctccaaaattgtttgtacaaaccgtaagaaatggcattaaacatgtacactgtt |
| | taccagctgaaacaaatgtttcatttaaaaaattcaatattgaagaattcggcaagacattggaaaacaattcttataa |
| | attcctaaaagacactgcaaaccataaaaacgctatgagctctgttgctactgatatgagttgtgatcatctcaaggg |
| | gagaagtaacgttttagtattccagcagcctggctttaactgcagttccattccacattcttcacactccatcataaatc |
| | atcatgctagtatacacaatgaaggtgatcaaccaaaaactcctgagaatataccaagtaaagaaccaaaagatgg |
| | atctcccgttcaaccaagtctcttatcgttaatgaaagataggagattaacattggagcaagtggtagccatagagg |
| | ccctgactcaactctcagaagccccatcagagaattcctccccatcaaagtcagagaaggatgaggaatcagagc |
| | agagaacagccagtttgcttaatagctgcaaagctatcctctacactgtaagaaaagacctccaagacccaaactt |
| | acagggagagccaccaaaacttaatcactgtccatctttggaaaaacaaagttcatgcaacacggtggttttcaatg |
| | ggcaaactactaccctttccaactcacatatcaactcagctactaaccaagcatccacaaagtcacatgaatattcaa |
| | aagtcacaaattcattatctctttttataccaaaatcaaattcatccaagattgacaccaataaaagtattgctcaaggg |
| | ataattactcttgacaattgttccaatgatttgcatcagttgccaccaagaaataatgaagtggagtattgcaaccagtt |
| | actggacagcagcaaaaaattggactcagatgatctatcatgtcaggatgcaacccatacccaaattgaggaagat |
| | gttgcaacacagttgacacaacttgcttcgataattaagatcaattatataaaaccagaggacaaaaaagttgaaag |
| | tacaccaacaagccttgtcacatgtaatgtacagcaaaaatacaatcaggagaagggcacaatacaacagaaacc |
| | accttcaagtgtacacaataatcatggttcatcattaacaaaacaaaagaacccaacccagaaaaagacaaaatcc |
| | accccatcaagagatcggcggaaaaagaagcccacagttgtaagttatcaagaaaatgatcggcagaagtggga |
| | aaagttgtcctatatgtatggcacaatatgcgacatttggatagcatcgaaatttcaaaattttgggcaattttgtccac |
| | atgattttcctactgtatttgggaaaatttcttcctcgaccaaaatatggaaaccactggctcaaacgaggtccattatg |
| | caacccaaaacagtatttccaccactcactcagataaaattacagagatatcctgaatcagcagaggaaaaggtga |
| | aggttgaaccattggattcactcagcttatttcatcttaaaacggaatccaacgggaaggcattcactgataaagctt |
| | ataattctcaggtacagttaacggtgaatgccaatcagaaagcccatcctttgacccagccctcctctccacctaac |
| | cagtgtgctaacgtgatggcaggcgatgaccaaatacggtttcagcaggttgttaaggagcaactcatgcatcaga |
| | gactgccaacattgcctggtatctctcatgaaacacccttaccggagtcagcactaactctcaggaatgtaaatgta |
| | gtgtgttcaggtggaattacagtggtttctaccaaaagtgaagaggaagtctgttcatccagttttggaacatcagaa |
| | ttttccacagtggacagtgcacagaaaaattttaatgattatgccatgaacttctttactaaccctacaaaaaacctagt |
| | gtctataactaaagattctgaactgcccacctgcagctgtcttgatcgagttatacaaaaagacaaaggcccatatta |
| | tacacaccttggggcaggaccaagtgttgctgctgtcagggaaatcatggagaataggtatggtcaaaaaggaaa |
| | cgcaataaggatagaaatagtagtgtacaccggtaaagaagggaaaagctctcatgggtgtccaattgctaagtg |
| | ggttttaagaagaagcagtgatgaagaaaaagttctttgtttggtccggcagcgtacaggccaccactgtccaact |
| | gctgtgatggtggtgctcatcatggtgtgggatggcatccctcttccaatggccgaccggctatacacagagctca |
| | cagagaatctaaagtcatacaatgggcaccctaccgacagaagatgcaccctcaatgaaaatcgtacctgtacat |
| | gtcaaggaattgatccagagacttgtggagcttcattctcttttggctgttcatggagtatgtactttaatggctgtaagt |
| | ttggtagaagcccaagccccagaagatttagaattgatccaagctctcccttacatgaaaaaaaccttgaagataac |
| | ttacagagtttggctacacgattagctccaatttataagcagtatgctccagtagcttaccaaaatcaggtggaatatg |
| | aaaatgttgcccgagaatgtcggcttggcagcaaggaaggtcgtcccttctctggggtcactgcttgcctggacttc |
| | tgtgctcatccccacagggacattcacaacatgaataatggaagcactgtggtttgtaccttaactcgagaagataa |
| | ccgctctttgggtgttattcctcaagatgagcagctccatgtgctacctctttataagctttcagacacagatgagtttg |
| | gctccaaggaaggaatggaagccaagatcaaatctggggccatcgaggtcctggcaccccgccgcaaaaaaa |
| | gaacgtgtttcactcagcctgttccccgttctggaaagaagagggctgcgatgatgacagaggttcttgcacataa |
| | gataagggcagtggaaaagaaacctattccccgaatcaagcggaagaataactcaacaacaacaaacaacagta |
| | agccttcgtcactgccaaccttagggagtaacactgagaccgtgcaacctgaagtaaaaagtgaaaccgaaccc |
| | cattttatcttaaaaagttcagacaacactaaaacttattcgctgatgccatccgctcctcacccagtgaaagaggca |
| | tctccaggcttctcctggtccccgaagactgcttcagccacaccagctccactgaagaatgacgcaacagcctcat |
| | gcgggttttcagaaagaagcagcactccccactgtacgatgccttcgggaagactcagtggtgccaatgcagctg |
| | ctgctgatggccctggcatttcacagcttggcgaagtggctcctctccccaccctgtctgctcctgtgatggagccc |
| | ctcattaattctgagccttccactggtgtgactgagccgctaacgcctcatcagccaaaccaccagccctccttcctc |
| | acctctcctcaagaccttgcctcttctccaatggaagaagatgagcagcattctgaagcagatgagcctccatcag |
| | acgaacccctatctgatgaccccctgtcacctgctgaggagaaattgccccacattgatgagtattggtcagacagt |
| | gagcacatctttttggatgcaaatattggtggggtggccatcgcacctgctcacggctcggttttgattgagtgtgcc |
| | cggcgagagctgcacgctaccactcctgttgagcaccccaaccgtaatcatccaacccgcctctcccttgtctttta |
| | ccagcacaaaaacctaaataagccccaacatggttttgaactaaacaagattaagtttgaggctaaagaagctaag |
| | aataagaaaatgaaggcctcagagcaaaaagaccaggcagctaatgaaggtccagaacagtcctctgaagtaaa |
| | tgaattgaaccaaattccttctcataaagcattaacattaacccatgacaatgttgtcaccgtgtccccttatgctctca |
| | cacacgttgcggggccctataaccattgggtctgaaggcttttctccccctcttaatgcctttgctagtgcagtgtattt |
| | tttcaaggtgctgttaaaagaaagtcatgttgtcgtttactatcttcatctcacccatttcaagtctgaggtaaaaaaata |
| | ataatgataacaaaacggggtgggtattcttaactgtgactatattttgacaattggtagaaggtgcacattttaagca |
| | aaaataaaagttttatagttttaaatacataaagaaatgtttcagttaggcattaaccttgatagaatcactcagtttggt |
| | gctttaaattaagtctgtttactatgaaacaagagtcatttttagaggattttaacaggttcatgttctatgatgtaaaat |
| | caagacacacagtgttaactctacacagcttctggtgcttaaccacatccacacagttaaaaataagctgaattattatt |
| | tcatggtgccattgttccaacatcttccaatcattgctagaaaattggcatattcctttgaaataaacttatgaaatgttt |
| | tctctcttaaaatatttctcctgtgtaaaataaatcattgttgttagtaatggttggaggctgttcataaattgtaaatat |
| | atattttaaaagcactttctatttttaaaagtaacttgaaataatatagtataagaatcctattgtctattgtttgtgcat |
| | atttgcatacaagagaaatcatttatccttgctgtgtagagttccatcttgttaactgcagtatgtattctaatcatgtat |
| | atggtttgtgttcttttactgtgtcctctcacattcaagtattagcaacttgcagtatataaaatagttagataatgagaa |
| | gttgttaattatctctaaaattggaattaggaagcatatcaccaatactgattaacattctctttggaactaggtaagagt |
| | ggtctcttcttattgaacaacctcaatttagtttcatcccacctttctcagtataatccatgagaggtgtttccaaaagga |
| | gatgagggaacaggataggtttcagaagagtcaaatgcttctaatgtctcaaggtgataaaatacaaaaactaagtagaca |
| | gatatttgtactgaagtctgatacagaattagaaaaaaaaaattcttgttgaaatattttgaaaacaaattccctactatc |
| | atcacatgcctccccaaccccaagtcaaaaacaagaggaatggtactacaaacatggctttgtccattaagagctaatt |
| | catttgtttatcttagcatactagatttgggaaaatgataactcatcttttctgataattgcctatgttctaggtaacagg |
| | aaaacaggcattaagtttattttagtcttcccattttcttcctattactttattgactcattttattgcaaaacaaaaagg |
| | attacccaaacaacatgtttcgaacaaggagaattttcaatgaaatacttgattctgttaaaatgcagaggtgctataaca |
| | ttcaaagtgtcagattccttgggagtatggaaaacctaatggtgcttctcccttggaaatgccataggaagcccacaa |
| | ccgctaacacttacaattttggtgcaaaagcaaacagttccagcaggctctctaaagaaaaactcattgtaacttatt |
| | aaaataatatctggtgcaaagtatctgttttgagcttttgactaatccaagtaaaggaatatgaagggattgtaaaaaa |
| | caaaatgtccattgatagaccatcgtgtacaagtagatttctgcttgttgaatatgtaaaatagggtaattcattgactt |
| | gttttagtattttgtgtgccttagatttccgttttaagacatgtatatttttgtgagcctaaggtttcttatatacatata |
| | agtatataaataagtgattgtttattgcttcagctgcttcaacaagatatttactagtattagactatcaggaatacaccc |
| | ttgcgagattatgttttagattttaggccttagctcccactagaaattatttcttcaccagatttaatggataaagtttta |
| | tggctctttatgcatccactcatctactcattcttcgagtctacacttattgaatgcctgcaaaatctaagtatcactttt |
| | atttttctttggatcaccacctatgacatagtaaacttgaagaataaaaactaccctcagaaatatttttaaaagaagtag |
| | caaattatcttcagtataatccatggtaatgtatgcagtaattcaaattgatctctctctcaataggtttcttaacaatct |
| | aaacttgaaacatcaatgttaatttttggaactattgggatttgtgacgcttgttgcagtttaccaaaacaagtatttgaa |
| | aatatatagtatcaactgaaatgtttccattccgttgttgtagttaacatcatgaatggacttcttaagctgattacccca |
| | ctgtgggaaccaaattggattcctactttgttggactctctttcctgattttaacaatttaccatcccattctctgccctg |
| | tgattttttttaaaagcttattcaatgttctgcagcattgtgattgtatgctggctacactgcttttagaatgctctttct |
| | catgaagcaaggaaataaatttgtttgaaatgacattttctctcataaaa |
| 3 | (Ac)YLIYAYYTWWEHSC |
| (molecule | |
| 36 Tip1) | |
| 4 | (Ac)YWYLPSYRVPWFC |
| (molecule | |
| 37 Tip2) | |
| 5 | (Ac)YYRVKTYYQITYVYLSC |
| (molecule | |
| 38 | |
| Tip3m15 | |
| L) | |

### REFERENCES

Throughout this application, various references describe the state of the art to which this invention pertains. The disclosures of these references are hereby incorporated by reference into the present disclosure.
Abbott, D. H., Dumesic, D. A., and Franks, S. (2002). Developmental origin of polycystic ovary syndrome—a hypothesis. The Journal of endocrinology 174, 1-5.
Anders, S., and Huber, W. (2010). Differential expression analysis for sequence count data. Genome Biol 11, R106.
Anders, S., Pyl, P. T., and Huber, W. (2015). HTSeq—a Python framework to work with high-throughput sequencing data. Bioinformatics 31, 166-169.
Azziz, R. (2016). PCOS in 2015: New insights into the genetics of polycystic ovary syndrome. Nature reviews. Endocrinology 12, 183.
Benjamini, Y. H., Y. (1995). Controlling the false discovery rate: a practical and powerful approach to multiple testing. J. R. Stat. Soc. Ser. B Methodol. 57, 289-300.
Bottiglieri, T. (2002). S-Adenosyl-L-methionine (SAMe): from the bench to the bedside—molecular basis of a pleiotrophic molecule. Am J Clin Nutr 76, 1151S-1157S.
Boyle, J. A., and Teede, H. J. (2016). PCOS: Refining diagnostic features in PCOS to optimize health outcomes. Nature reviews. Endocrinology 12, 630-631.
Caldwell, A. S. L., Edwards, M. C., Desai, R., Jimenez, M., Gilchrist, R. B., Handelsman, D. J., and Walters, K. A. (2017). Neuroendocrine androgen action is a key extraovarian mediator in the development of polycystic ovary syndrome. Proceedings of the National Academy of Sciences of the United States of America 114, E3334-E3343.
Cavalli, G., and Heard, E. (2019). Advances in epigenetics link genetics to the environment and disease. Nature 571, 489-499.
Chen, M. J., Yang, W. S., Chen, H. F., Kuo, J. J., Ho, H. N., Yang, Y. S., and Chen, S. U. (2010). Increased follistatin levels after oral contraceptive treatment in obese and non-obese women with polycystic ovary syndrome. Human reproduction 25, 779-785.
Chik, F., Machnes, Z., and Szyf, M. (2014). Synergistic anti-breast cancer effect of a combined treatment with the methyl donor S-adenosyl methionine and the DNA methylation inhibitor 5-aza-2′-deoxycytidine. Carcinogenesis 35, 138-144.
Crisosto, N., Codner, E., Maliqueo, M., Echiburu, B., Sanchez, F., Cassorla, F., and Sir-Petermann, T. (2007). Anti-Mullerian hormone levels in peripubertal daughters of women with polycystic ovary syndrome. The Journal of clinical endocrinology and metabolism 92 , 2739-2743.
Crisosto, N., Ladron de Guevara, A., Echiburu, B., Maliqueo, M., Cavada, G., Codner, E., Paez, F., and Sir-Petermann, T. (2019). Higher luteinizing hormone levels associated with antimullerian hormone in postmenarchal daughters of women with polycystic ovary syndrome. Fertility and sterility 111, 381-388.
Deaton, A. M., and Bird, A. (2011). CpG islands and the regulation of transcription. Genes Dev 25, 1010-1022.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Dokras, A., Saini, S., Gibson-Helm, M., Schulkin, J., Cooney, L., and Teede, H. (2017). Gaps in knowledge among physicians regarding diagnostic criteria and management of polycystic ovary syndrome. Fertility and sterility 107, 1380-1386 e1381.
Dumesic, D. A., Oberfield, S. E., Stener-Victorin, E., Marshall, J. C., Laven, J. S., and Legro, R. S. (2015). Scientific Statement on the Diagnostic Criteria, Epidemiology, Pathophysiology, and Molecular Genetics of Polycystic Ovary Syndrome. Endocrine reviews 36, 487-525.
Dupont, J., and Scaramuzzi, R. J. (2016). Insulin signalling and glucose transport in the ovary and ovarian function during the ovarian cycle. The Biochemical journal 473, 1483-1501.
Escobar-Morreale, H. F. (2018). Polycystic ovary syndrome: definition, aetiology, diagnosis and treatment. Nature reviews. Endocrinology 14, 270-284.
Findlay, J. K. (1993). An update on the roles of inhibin, activin, and follistatin as local regulators of folliculogenesis. Biol Reprod 48, 15-23.
Franks, S. (2008). Polycystic ovary syndrome in adolescents. International journal of obesity 32, 1035-1041.
Franks, S., and Berga, S. L. (2012). Does PCOS have developmental origins? Fertility and sterility 97, 2-6.
Gao, L., Gu, Y., and Yin, X. (2016). High Serum Tumor Necrosis Factor-Alpha Levels in Women with Polycystic Ovary Syndrome: A Meta-Analysis. PloS one 11, e0164021.
Gapp, K., von Ziegler, L., Tweedie-Cullen, R. Y., and Mansuy, I. M. (2014). Early life epigenetic programming and transmission of stress-induced traits in mammals: how and when can environmental factors influence traits and their transgenerational inheritance? Bioessays 36, 491-502.
Gonzalez, F., Rote, N. S., Minium, J., and Kirwan, J. P. (2006). Increased activation of nuclear factor kappaB triggers inflammation and insulin resistance in polycystic ovary syndrome. The Journal of clinical endocrinology and metabolism 91, 1508-1512.
Gorsic, L. K., Dapas, M., Legro, R. S., Hayes, M. G., and Urbanek, M. (2019). Functional Genetic Variation in the Anti-Mullerian Hormone Pathway in Women With Polycystic Ovary Syndrome. The Journal of clinical endocrinology and metabolism 104 , 2855-2874.
Gorsic, L. K., Kosova, G., Werstein, B., Sisk, R., Legro, R. S., Hayes, M. G., Teixeira, J. M., Dunaif, A., and Urbanek, M. (2017). Pathogenic Anti-Mullerian Hormone Variants in Polycystic Ovary Syndrome. The Journal of clinical endocrinology and metabolism 102 , 2862-2872.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Ignatiadis, N., Klaus, B., Zaugg, J. B., and Huber, W. (2016). Data-driven hypothesis weighting increases detection power in genome-scale multiple testing. Nat Methods 13, 577-580.
Jiang, L., Huang, J., Li, L., Chen, Y., Chen, X., Zhao, X., and Yang, D. (2015). MicroRNA-93 promotes ovarian granulosa cells proliferation through targeting CDKN1A in polycystic ovarian syndrome. The Journal of clinical endocrinology and metabolism 100, E729-738.
Kelly, T. K., De Carvalho, D. D., and Jones, P. A. (2010). Epigenetic modifications as therapeutic targets. Nat Biotechnol 28, 1069-1078.
Lambertini, L., Saul, S. R., Copperman, A. B., Hammerstad, S. S., Yi, Z., Zhang, W., Tomer, Y., and Kase, N. (2017). Intrauterine Reprogramming of the Polycystic Ovary Syndrome: Evidence from a Pilot Study of Cord Blood Global Methylation Analysis. Frontiers in endocrinology 8, 352.
Lang, Q., Yidong, X., Xueguang, Z., Sixian, W., Wenming, X., and Tao, Z. (2019). ETA-mediated anti-TNF-alpha therapy ameliorates the phenotype of PCOS model induced by letrozole. PloS one 14, e0217495.
Langmead, B., Trapnell, C., Pop, M., and Salzberg, S. L. (2009). Ultrafast and memory-efficient alignment of short DNA sequences to the human genome. Genome Biol 10, R25.
Leek, J. T. (2014). svaseq: removing batch effects and other unwanted noise from sequencing data. Nucleic Acids Res 42.
Li, T. W., Yang, H., Peng, H., Xia, M., Mato, J. M., and Lu, S. C. (2012). Effects of S-adenosylmethionine and methylthioadenosine on inflammation-induced colon cancer in mice. Carcinogenesis 33, 427-435.
Liu, M., Gao, J., Zhang, Y., Li, P., Wang, H., Ren, X., and Li, C. (2015). Serum levels of TSP-1, NF-kappaB and TGF-beta1 in polycystic ovarian syndrome (PCOS) patients in northern China suggest PCOS is associated with chronic inflammation. Clinical endocrinology 83, 913-922.
Liu, Q., Li, Y., Feng, Y., Liu, C., Ma, J., Li, Y., Xiang, H., Ji, Y., Cao, Y., Tong, X., et al. (2016). Single-cell analysis of differences in transcriptomic profiles of oocytes and cumulus cells at GV, MI, MII stages from PCOS patients. Sci Rep 6, 39638.
Livak, K. J., and Schmittgen, T. D. (2001). Analysis of relative gene expression data using real-time quantitative PCR and the 2(−Delta Delta C(T)) Method. Methods 25, 402-408.
Love, MI., Huber, W., and Anders, S. (2014). Moderated estimation of fold change and dispersion for RNA-seq data with DESeq2. Genome Biol 15, 550.
Makrinou, E., Drong, A. W., Christopoulos, G., Lerner, A., Chapa-Chorda, I., Karaderi, T., Lavery, S., Hardy, K., Lindgren, C. M., and Franks, S. (2020). Genome-wide methylation profiling in granulosa lutein cells of women with polycystic ovary syndrome (PCOS). Molecular and cellular endocrinology 500, 110611.
March, W. A., Moore, V. M., Willson, K. J., Phillips, D. I., Norman, R. J., and Davies, M. J. (2010). The prevalence of polycystic ovary syndrome in a community sample assessed under contrasting diagnostic criteria. Human reproduction 25, 544-551.
Martin, M., et al. (2011). Cutadapt removes adapter sequences from high-throughput sequencing reads. . EMBnet.journal 17, 10-12.
McAllister, J. M., Legro, R. S., Modi, B. P., and Strauss, J. F., 3rd (2015). Functional genomics of PCOS: from GWAS to molecular mechanisms. Trends in endocrinology and metabolism: TEM 26, 118-124.
Moore, A. M., Prescott, M., Marshall, C. J., Yip, S. H., and Campbell, R. E. (2015). Enhancement of a robust arcuate GABAergic input to gonadotropin-releasing hormone neurons in a model of polycystic ovarian syndrome. Proceedings of the National Academy of Sciences of the United States of America 112, 596-601.
Padmanabhan, V., and Veiga-Lopez, A. (2013). Sheep models of polycystic ovary syndrome phenotype. Molecular and cellular endocrinology 373, 8-20.
Pan, J. X., Tan, Y. J., Wang, F. F., Hou, N. N., Xiang, Y. Q., Zhang, J. Y., Liu, Y., Qu, F., Meng, Q., Xu, J., et al. (2018). Aberrant expression and DNA methylation of lipid metabolism genes in PCOS: a new insight into its pathogenesis. Clin Epigenetics 10, 6.
Patel, S. (2018). Polycystic ovary syndrome (PCOS), an inflammatory, systemic, lifestyle endocrinopathy. The Journal of steroid biochemistry and molecular biology 182, 27-36.
Pigny, P., Desailloud, R., Cortet-Rudelli, C., Duhamel, A., Deroubaix-Allard, D., Racadot, A., and Dewailly, D. (1997). Serum alpha-inhibin levels in polycystic ovary syndrome: relationship to the serum androstenedione level. The Journal of clinical endocrinology and metabolism 82, 1939-1943.
Piltonen, T. T., Giacobini, P., Edvinsson, A., Hustad, S., Lager, S., Morin-Papunen, L., Tapanainen, J. S., Sundstrom-Poromaa, I., and Arffman, R. K. (2019). Circulating antimullerian hormone and steroid hormone levels remain high in pregnant women with polycystic ovary syndrome at term. Fertility and sterility 111, 588-596 e581.
Poulsen, L. C., Englund, A. L. M., Andersen, A. S., Botkjaer, J. A., Mamsen, L. S., Damdimopoulou, P., Ostrup, O., Grondahl, M. L., and Andersen, C. Y. (2020). Follicular hormone dynamics during the midcycle surge of gonadotropins in women undergoing fertility treatment. Mol Hum Reprod.
Qi, X., Yun, C., Sun, L., Xia, J., Wu, Q., Wang, Y., Wang, L., Zhang, Y., Liang, X., Wang, L., et al. (2019). Gut microbiota-bile acid-interleukin-22 axis orchestrates polycystic ovary syndrome. Nat Med 25, 1225-1233.
Qu, F., Wang, F. F., Yin, R., Ding, G. L., El-Prince, M., Gao, Q., Shi, B. W., Pan, H. H., Huang, Y. T., Jin, M., et al. (2012). A molecular mechanism underlying ovarian dysfunction of polycystic ovary syndrome: hyperandrogenism induces epigenetic alterations in the granulosa cells. J Mol Med (Berl) 90, 911-923.
Quinlan, A. R., and Hall, I. M. (2010). BEDTools: a flexible suite of utilities for comparing genomic features. Bioinformatics 26, 841-842.
Richards, J. S., and Pangas, S. A. (2010). The ovary: basic biology and clinical implications. The Journal of clinical investigation 120, 963-972.
Risal, S., Pei, Y., Lu, H., Manti, M., Fornes, R., Pui, H. P., Zhao, Z., Massart, J., Ohlsson, C., Lindgren, E., et al. (2019). Prenatal androgen exposure and transgenerational susceptibility to polycystic ovary syndrome. Nat Med 25, 1894-1904.
Roland, A. V., Nunemaker, C. S., Keller, S. R., and Moenter, S. M. (2010). Prenatal androgen exposure programs metabolic dysfunction in female mice. The Journal of endocrinology 207, 213-223.
Rotterdam, E. A.-S. P. c. w. g. (2004). Revised 2003 consensus on diagnostic criteria and long-term health risks related to polycystic ovary syndrome (PCOS). Human reproduction 19, 41-47.
Schulze, S. K., Kanwar, R., Golzenleuchter, M., Therneau, T. M., and Beutler, A. S. (2012). SERE: single-parameter quality control and sample comparison for RNA-Seq. BMC Genomics 13, 524.
Shen, H. R., Qiu, L. H., Zhang, Z. Q., Qin, Y. Y., Cao, C., and Di, W. (2013). Genome-wide methylated DNA immunoprecipitation analysis of patients with polycystic ovary syndrome. PloS one 8, e64801.
Sir-Petermann, T., Ladron de Guevara, A., Codner, E., Preisler, J., Crisosto, N., Echiburu, B., Maliqueo, M., Sanchez, F., Perez-Bravo, F., and Cassorla, F. (2012). Relationship between anti-Mullerian hormone (AMH) and insulin levels during different tanner stages in daughters of women with polycystic ovary syndrome. Reproductive sciences (Thousand Oaks, Calif.) 19, 383-390.
Sir-Petermann, T., Maliqueo, M., Angel, B., Lara, H. E., Perez-Bravo, F., and Recabarren, S. E. (2002). Maternal serum androgens in pregnant women with polycystic ovarian syndrome: possible implications in prenatal androgenization. Human reproduction 17 , 2573-2579.
Sjoholm, A., and Nystrom, T. (2006). Inflammation and the etiology of type 2 diabetes. Diabetes Metab Res Rev 22, 4-10.
Stener-Victorin, E., Padmanabhan, V., Walters, K. A., Campbell, R. E., Benrick, A., Giacobini, P., Dumesic, D. A., and Abbott, D. H. (2020). Animal Models to Understand the Etiology and Pathophysiology of Polycystic Ovary Syndrome. Endocrine reviews 41.
Stener-Victorin, E., Ploj, K., Larsson, B. M., and Holmang, A. (2005). Rats with steroid-induced polycystic ovaries develop hypertension and increased sympathetic nervous system activity. Reproductive biology and endocrinology: RB&E 3, 44.
Steyn, F. J., Wan, Y., Clarkson, J., Veldhuis, J. D., Herbison, A. E., and Chen, C. (2013). Development of a methodology for and assessment of pulsatile luteinizing hormone secretion in juvenile and adult male mice. Endocrinology 154, 4939-4945.
Sullivan, S. D., and Moenter, S. M. (2004). Prenatal androgens alter GABAergic drive to gonadotropin-releasing hormone neurons: implications for a common fertility disorder. Proceedings of the National Academy of Sciences of the United States of America 101, 7129-7134.
Tata, B., Mimouni, N. E. H., Barbotin, A. L., Malone, S. A., Loyens, A., Pigny, P., Dewailly, D., Catteau-Jonard, S., Sundstrom-Poromaa, I., Piltonen, T. T., et al. (2018). Elevated prenatal anti-Mullerian hormone reprograms the fetus and induces polycystic ovary syndrome in adulthood. Nat Med.
Tremblay, M. W., and Jiang, Y. H. (2019). DNA Methylation and Susceptibility to Autism Spectrum Disorder. Annu Rev Med 70, 151-166. Vazquez-Martinez, E. R., Gomez-Viais, Y. I., Garcia-Gomez, E., Reyes-Mayoral, C., Reyes-Munoz, E., Camacho-Arroyo, I., and Cerbon, M. (2019). DNA methylation in the pathogenesis of polycystic ovary syndrome. Reproduction 158, R27-R40.
Wachs, D. S., Coffler, M. S., Malcom, P. J., and Chang, R. J. (2006). Comparison of follicle-stimulating-hormone-stimulated dimeric inhibin and estradiol responses as indicators of granulosa cell function in polycystic ovary syndrome and normal women. The Journal of clinical endocrinology and metabolism 91, 2920-2925.
Walters, K. A., Bertoldo, M. J., and Handelsman, D. J. (2018a). Evidence from animal models on the pathogenesis of PCOS. Best Pract Res Clin Endocrinol Metab 32, 271-281.
Walters, K. A., Gilchrist, R. B., Ledger, W. L., Teede, H. J., Handelsman, D. J., and Campbell, R. E. (2018b). New Perspectives on the Pathogenesis of PCOS: Neuroendocrine Origins. Trends in endocrinology and metabolism: TEM 29, 841-852.
Wang, K., You, L., Shi, Y., Wang, L., Zhang, M., and Chen, Z. J. (2008). Association of genetic variants of insulin degrading enzyme with metabolic features in women with polycystic ovary syndrome. Fertility and sterility 90, 378-384.
Wang, L., Wang, S., and Li, W. (2012). RSeQC: quality control of RNA-seq experiments. Bioinformatics 28, 2184-2185.
Wang, X. X., Wei, J. Z., Jiao, J., Jiang, S. Y., Yu, D. H., and Li, D. (2014). Genome-wide DNA methylation and gene expression patterns provide insight into polycystic ovary syndrome development. Oncotarget 5, 6603-6610.
Wild, R. A., Carmina, E., Diamanti-Kandarakis, E., Dokras, A., Escobar-Morreale, H. F., Futterweit, W., Lobo, R., Norman, R. J., Talbott, E., and Dumesic, D. A. (2010). Assessment of cardiovascular risk and prevention of cardiovascular disease in women with the polycystic ovary syndrome: a consensus statement by the Androgen Excess and Polycystic Ovary Syndrome (AE-PCOS) Society. The Journal of clinical endocrinology and metabolism 95, 2038-2049.
Xiong, Z., Li, B., Wang, L., Zeng, X., Li, B., Sha, X., and Liu, H. (2019). AQP8 and AQP9 expression in patients with polycystic ovary syndrome and its association with in vitro fertilization-embryo transfer outcomes. Exp Ther Med 18, 755-760.
Xu, J., Bao, X., Peng, Z., Wang, L., Du, L., Niu, W., and Sun, Y. (2016). Comprehensive analysis of genome-wide DNA methylation across human polycystic ovary syndrome ovary granulosa cell. Oncotarget 7, 27899-27909.
Xu, N., Azziz, R., and Goodarzi, M. O. (2010). Epigenetics in polycystic ovary syndrome. a pilot study of global DNA methylation. Fertility and sterility 94, 781-783 e781.
Yu, Y. Y., Sun, C. X., Liu, Y. K., Li, Y., Wang, L., and Zhang, W. (2015). Genome-wide screen of ovary-specific DNA methylation in polycystic ovary syndrome. Fertility and sterility 104, 145-153 e146.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol 9, R137.
Zhao, Y., Zhang, C., Huang, Y., Yu, Y., Li, R., Li, M., Liu, N., Liu, P., and Qiao, J. (2015). Up-regulated expression of WNT5a increases inflammation and oxidative stress via PI3K/AKT/NF-kappaB signaling in the granulosa cells of PCOS patients. The Journal of clinical endocrinology and

1. A method for assessing a subject's risk of having or developing Polycystic Ovary Syndrome (PCOS) and treating the subject, comprising i) determining in a sample obtained from the subject a methylation status of one or more genes selected from a group consisting of: TET1, ROBO1, HDC, IGFBPL1, CDKN1A and IRS4, ii) treating a subject identified as having a methylation status of at least one of the one or more genes that is lower than a corresponding reference value with a methylating agent and/or a TET1 inhibitor.

2. The method according to claim 1, wherein the sample is a blood sample.

3. The method according to claim 1, wherein the methylation status of one, two, three, four five or six of the one or more genes is determined.

4. An in vitro method for monitoring a Polycystic Ovary Syndrome (PCOS) in a subject in need thereof, comprising i) determining a methylation status of one or more genes selected from the group consisting of: TET1, ROBO1, HDC, IGFBPL1, CDKN1A and IRS4 in a sample obtained from the subject at a first specific time of the disease, ii) determining the methylation status of the one or more genes in a sample obtained from the subject at a second specific time of the disease, iii) comparing the methylation status determined at step i) with the methylation status determined at step ii) and iv) concluding that the disease has evolved in a better manner when the methylation status of at least one of the one or more genes determined at step ii) is higher than the methylation status determined at step i).

5. An in vitro method for monitoring the treatment of Polycystic Ovary Syndrome (PCOS) in a subject in need thereof, comprising i) determining a methylation status of one or more genes selected from the group consisting of: TET1, ROBO1, HDC, IGFBPL1, CDKN1A and IRS4 in a sample obtained from the subject before the treatment, ii) determining the methylation status of the one or more genes in a sample obtained from the subject after the treatment, iii) comparing the methylation status determined at step i) with the methylation status determined at step ii) and iv) concluding that the treatment is efficient when the methylation status determined at step ii) is higher than the methylation status determined at step i).

6. The in vitro method according to claim 4, wherein the sample is a blood sample.

7. A method of preventing or treating Polycystic Ovary Syndrome (PCOS) in a subject in need thereof, comprising
administering to the subject a therapeutically effective amount of a methylating agent.

8. A method of preventing or treating Polycystic Ovary Syndrome (PCOS) in a subject in need thereof, comprising
administering to the subject a therapeutically effective amount of a TET1 inhibitor

9. The method according to claim 8 wherein the TET1 inhibitor is selected from:
a) an inhibitor of TET1 activity
and/or
b) an inhibitor of TET1 gene expression.

10. The method according to claim 9 wherein said inhibitor of TET1 activity is selected from the group consisting of an antibody, a peptide, an aptamer, and a small organic molecule.

11. The method according to claim 9 wherein the inhibitor of TET1 gene expression is selected from the group consisting of an antisense oligonucleotide, a nuclease, siRNA, shRNA, and a ribozyme nucleic acid sequence.

12. The method according to claim 8, further further comprising, prior to the step of administering, determining a methylation status of one or more genes selected from the group consisting of: TET1, ROBO1, HDC, IGFBPL1, CDKN1A and IRS4 by the method of claim 1.

13. The method according to claim 12, wherein the biological sample is a blood sample.

14. (canceled)
